(19) United States
(12) Patent Application Publication
(10) Pub. No.: US 2004/0229900 A1
(43) Pub. Date: Nov. 18, 2004

(54) Bispidine compounds useful in the treatment of cardiac arrythmias

(75) Inventors: Christer Alstermark, Molndal (SE); Kjell Andersson, Fjaras (SE); Annika Bjore, Stenungsund (SE); Magnus Bjorsne, Vastra Frolunda (SE); Eva-Lotte Lindstedt Alstermark, Molndal (SE); Goran N. Nilsson, Henan (SE); Magnus Polla, Gothenburg (SE); Gert Strandlund, Lindome (SE); Yiva Ortengren, Onsala (SE)

Correspondence Address: NIXON & VANDERHYE, PC, 1100 N GLEBE ROAD, 8TH FLOOR, ARLINGTON, VA, 22201-4714, US

(73) Assignee: AstraZeneca AB, Sodertalje (SE)

(21) Appl. No.: 10/871,022

(22) Filed: Jun. 21, 2004

Related U.S. Application Data
(63) Continuation of application No. 09/623,726, filed on Sep. 7, 2000, now abandoned.
Application No. 09/623,726 is a 371 of international application No. PCT/SE00/01254, filed on Jun. 15, 2000.

(30) Foreign Application Priority Data
Jun. 16, 1999 (SE) 9902268-3

(51) Int. Cl. C07D 471/02

(52) U.S. Cl. 514/300; 546/113

(57) ABSTRACT

There is provided compounds of formula I,

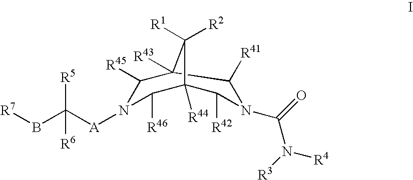

wherein R¹, R², R³, R⁴, R⁵, R⁶, R⁷, R⁴¹, R⁴², R⁴³ R⁴⁴, R⁴⁵, R⁴⁶, A and B have meanings given in the description, which are useful in the prophylaxis and in the treatment of arrhythmias, in particular atrial and ventricular arrhythmias.

### FIELD OF THE INVENTION

This invention relates to novel pharmaceutically useful compounds, in particular compounds which are useful in the treatment of cardiac arrhythmias.

### BACKGROUND AND PRIOR ART

Cardiac arrhythmias may be defined as abnormalities in the rate, regularity, or site of origin of the cardiac impulse or as disturbances in conduction which causes an abnormal sequence of activation. Arrhythmias may be classified clinically by means of the presumed site of origin (i.e. as supraventricular, including atrial and atrioventricular, arrhythmias and ventricular arrhythmias) and/or by means of rate (i.e. bradyarrhythmias (slow) and tachyarrhythmias (fast)).

In the treatment of cardiac arrhythmias, the negative outcome in clinical trials (see, for example, the outcome of the Cardiac Arrhythmia Suppression Trial (CAST) reported in New England Journal of Medicine, 321, 406 (1989)) with “traditional” antiarrhythmic drugs, which act primarily by slowing the conduction velocity (class I antiarrhythmic drugs), has prompted drug development towards compounds which selectively delay cardiac repolarization, thus prolonging the QT interval. Class III antiarrhythmic drugs may be defined as drugs which prolong the trans-membrane action potential duration (which can be caused by a block of outward K⁺ currents or from an increase of inward ion currents) and refractoriness, without affecting cardiac conduction.

One of the key disadvantages of hitherto known drugs which act by delaying repolarization (class III or otherwise) is that they all are known to exhibit a unique form of proarrhythmia known as torsades de pointes (turning of points), which may, on occasion be fatal. From the point of view of safety, the minimisation of this phenomenon (which has also been shown to be exhibited as a result of administration of non-cardiac drugs such as phenothiazines, tricyclic antidepressants, antihistamines and antibiotics) is a key problem to be solved in the provision of effective antiarrhythmic drugs.

Antiarrhythmic drugs based on bispidines (3,7-diazabicyclo[3.3.1]nonanes), are known from inter alia international patent application WO 91/07405, European patent applications 306 871, 308 843 and 655 228 and U.S. Pat. Nos. 3,962,449, 4,556,662, 4,550,112, 4,459,301 and 5,468,858, as well as journal articles including inter alia J. Med. Chem. 39, 2559, (1996), Pharmacol. Res., 24, 149 (1991), Circulation, 90, 2032 (1994) and Anal. Sci. 9, 429, (1993). Known bispidine-based antiarrhythmic compounds include bisaramil (3-methyl-7-ethyl-9α,4′-(Cl-benzoyloxy)-3,7-diazabicyclo[3.3.1]nonane), tedisamil (3′,7′-bis(cyclopropylmethyl)spiro-(cyclopentane-1,9′)-3,7-diazabicyclo[3.3.1]nonane), SAZ-VII-22 (3-(4-chlorobenzoyl)-7-iso-propyl-3,7-diazabicyclo[3.3.1]nonane), SAZ-VII-23 (3-benzoyl-7-iso-propyl-3,7-diazabicyclo[3.3.1]nonane), GLG-V-13 (3-[4-(1H-imidazol-1-yl)benzoyl]-7-iso-propyl-3,7-diazabicyclo[3.3.1]nonane), KMC-IV-84 (7-[4′-(1H-imidazolo-1-yl)benzenesulfonyl]-3-iso-propyl-3,7-diazabicyclo[3.3.1]nonane dihydroperchlorate and ambasilide (3-(4-aminobenzoyl)-7-benzyl-3,7-diazabicyclo[3.3.1]nonane).

We have surprisingly found that a novel group of bispidine-based compounds exhibit electrophysiological activity, preferably class III electrophysiological activity, and are therefore expected to be useful in the treatment of cardiac arrhythmias.

### DISCLOSURE OF THE INVENTION

According to the invention there is provided compounds of formula I,

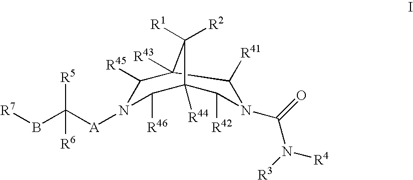

wherein

R¹ and R² independently represent H, C₁₋₄ alkyl, OR^{2b} or N(R^{2c})R^{2d}, or together form —O—(CH₂)₂—O—, —(CH₂)₃—, —(CH₂)₄— or —(CH₂)₅—;

R^{2b}, R^{2c} and R^{2d} independently represent H or C₁₋₆ alkyl;

R³ represents H, C₁₋₆ alkyl or, together with R⁴, represents C₃₋₆ alkylene (which alkylene group is optionally interrupted by O atom and/or is optionally substituted by one or more C₁₋₃ alkyl groups);

R⁴ represents H, C₁₋₁₂ alkyl, C₁₋₆ alkoxy (which latter two groups are both optionally substituted and/or terminated by one or more substituents selected from —OH, halo, cyano, nitro, C₁₋₄ alkyl and/or C₁₋₄ alkoxy), —(CH₂)_{q}-aryl, —(CH₂)_{q}-oxyaryl, —(CH₂)_{q}-Het¹ (which latter three groups are optionally substituted (at the —(CH₂)_{q}— part and/or the aryl/Het¹ part) by one or more substituents selected from —OH, halo, cyano, nitro, —C(O)R¹⁰, —C(O)OR¹¹, —N(H)S(O)₂R^{11a}, C₁₋₆ alkyl and/or C₁₋₆ alkoxy), —(CH₂)_{q}N(H)C(O)R⁸, —(CH₂)_{q}S(O)₂R⁸, —(CH₂)_{q}C(O)R⁸, —(CH₂)_{q}C(O)OR⁸, —(CH₂)_{q}C(O)N(R⁹)R⁸ or, together with R³, represents C₃₋₆ alkylene (which alkylene group is optionally interrupted by O atom and/or is optionally substituted by one or more C₁₋₃ alkyl groups);

q represents 0, 1, 2, 3, 4, 5 or 6;

R⁸ represents H, C₁₋₆, alkyl, aryl (which latter group is optionally substituted and/or terminated by one or more substituents selected from —OH, halo, cyano, nitro, —C(O)R¹⁰, —C(O)OR¹¹, —N(H)S(O)₂R^{11a}, C₁₋₆ alkyl and/or C₁₋₆ alkoxy) or, together with R⁹, represents C₃₋₇ alkylene;

R⁹ represents H, C₁₋₄ alkyl or, together with R⁸, represents C₃₋₇ alkylene;

Het¹ represents a five to twelve-membered heterocyclic ring containing one or more heteroatoms selected from oxygen, nitrogen and/or sulfur, and which also optionally includes one or more ═O substituents;

R⁴¹, R⁴², R⁴³, R⁴⁴, R⁴⁵ or R⁴⁶ independently represent H or C₁₋₃ alkyl;

R⁵ represents H, halo, C₁₋₃ alkyl, —OR¹², —N(R¹³)R¹² or, together with R⁶, represents ═O;

R⁶ represents H, C₁₋₄ alkyl or, together with R⁵, represents ═O;

R¹² represents H, C₁₋₆ alkyl, —S(O)₂—C₁₋₄-alkyl, —C(O)R¹⁴, —C(O)OR¹⁴, —C(O)N(R¹⁵)R^{15a} or aryl (which latter group is optionally substituted and/or terminated by one or more substituents selected from —OH, halo, cyano, nitro, —C(O)R¹⁰, —C(O)OR¹¹, —N(H)S(O)₂R^{11a}, C₁₋₆ alkyl and/or C₁₋₆ alkoxy);

R¹³ represents H or C₁₋₄ alkyl;

R¹⁴ represents H or C₁₋₆ alkyl;

R¹⁵ and R^{15a} independently represent H or C₁₋₄ alkyl, or together represent C₃₋₆ alkylene, optionally interrupted by O atom;

A represents a single bond, C₁₋₆ alkylene, —N(R¹⁶)(CH₂)ᵣ— or —O(CH₂)ᵣ— (in which two latter groups, the —(CH₂)ᵣ— group is attached to the bispidine nitrogen atom);

B represents a single bond, C₁₋₄ alkylene, —(CH₂)ₙN(R¹⁷)—, —(CH₂)ₙS(O)ₚ—, —(CH₂)ₙO— (in which three latter groups, the —(CH₂)ₙ— group is attached to the carbon atom bearing R⁵ and R⁶), —C(O)N(R²⁷)— (in which latter group, the —C(O)— group is attached to the carbon atom bearing R⁵ and R⁶), —N(R¹⁷)C(O)O(CH₂)ₙ—, —N(R¹⁷)(CH₂)ₙ— (in which two latter groups, the N(R⁷) group is attached to the carbon atom bearing R⁵ and R⁶) or —(CH₂)ₘC(H)(OH)(CH₂)ₙ— (in which latter group, the —(CH₂)ₘ— group is attached to the carbon atom bearing R⁵ and R⁶);

m represents 1, 2 or 3;

n and r independently represent 0, 1, 2, 3 or 4;

p represents 0, 1 or 2;

R¹⁶ and R¹⁷ independently represent H or C₁₋₄ alkyl;

R⁷ represents C₁₋₆ alkyl, aryl or Het², all of which groups are optionally substituted and/or terminated (as appropriate) by one or more substituents selected from —OH, cyano, halo, amino, nitro, Het³, —C(O)R¹⁰, —C(O)OR¹¹, C₁₋₆ alkyl, C₁₋₆ alkoxy, —N(H)S(O)₂R¹⁸, —S(O)₂R¹⁹, —OS(O)₂R²⁰, —N(H)C(O)N(H)R²¹, —C(O)N(H)R²² and/or aryl (which latter group is optionally substituted by one or more cyano groups);

Het² and Het³ independently represent a five to twelve-membered heterocyclic group containing one or more heteroatoms selected from oxygen, nitrogen and/or sulfur, and which also optionally includes one or more ═O substituents;

R¹⁸, R¹⁹ and R²⁰ independently represent C₁₋₆ alkyl;

R²¹ and R²² independently represent H or C₁₋₆ alkyl (optionally terminated by cyano); and

R¹⁰ and R¹¹ independently represent, at each individual occurrence, H or C₁₋₆ alkyl;

R^{11a} represents, at each individual occurrence, C₁₋₆ alkyl;

or a pharmaceutically acceptable derivative thereof;

provided that:

(a) when A and B are both single bonds and R⁷ is optionally substituted aryl, then R⁵ and R⁶ do not both represent H;

(b) when A represents a single bond, then R⁵ and R⁶ do not together represent ═O; and

(c) when R⁵ represents —OR¹² or —N(R¹³)R¹², then:

(i) A does not represent —N(R¹⁶)(CH₂)ᵣ— or —O(CH₂)ᵣ—; and/or

(ii) n does not represent 0 when B represents —(CH₂)ₙN(R¹⁷)—, —(CH₂)ₙS(O)ₚ— or —(CH₂)ₙO—,

which compounds are referred to hereinafter as “the compounds of the invention”.

Aryl groups that may be mentioned include C₆₋₁₀ aryl groups, such as phenyl, naphthyl and the like. Oxyaryl groups that may be mentioned include C₆₋₁₀ oxyaryl groups, such as oxyphenyl (phenoxy), oxynaphthyl (naphthoxy) and the like. When substituted, aryl and aryloxy groups are preferably substituted by one to three substituents.

Het¹, Het² and Het³ groups that may be mentioned include those containing 1 to 4 heteroatoms (selected from the group oxygen, nitrogen and/or sulfur) and in which the total number of atoms in the ring system are between five and twelve. Het (Het¹, Het² and Het³) groups may be wholly/partly aromatic in character and may be bicyclic. Heterocyclic groups that may be mentioned include morpholinyl, thiazolyl, oxazolyl, isoxazolyl, cinnolinyl, quinazolinyl, phthalazinyl, purinyl, benzimidazolyl, pyrimindinyl, piperazinyl, pyrazinyl, piperidinyl, pyridinyl, triazolyl, imidazolyl, quinolinyl, isoquinolinyl, dioxanyl, benzodioxanyl, benzodioxolyl, benzodioxepanyl, benzomorpholinyl, indolyl, pyrazolyl, pyrrolyl, benzothiophenyl, thiophenyl, chromanyl, thiochromanyl, benzofuranyl, pyranyl, tetrahydropyranyl, tetrahydrofuranyl, furanyl and the like. Values of Het¹ that may be mentioned include tetrahydropyranyl, isoxazolyl, benzodioxolyl, benzodioxepanyl and thiophenyl. Values of Het² that may be mentioned include quinolinyl, isoquinolinyl, benzomorpholinyl, benzodioxanyl, piperazinyl, indolyl and pyrazolyl. Values of Het³ that may be mentioned include imidazolyl. Substituents on Het (Het¹, Het² and Het³) groups may, where appropriate, be located on any atom in the ring system including a heteroatom. The point of attachment of Het (Het¹, Het² and Het³) groups may be via any atom in the ring system including (where appropriate) a heteroatom. Het (Het¹, Het² and Het³) groups may also be in the N- or S-oxidised form.

Pharmaceutically acceptable derivatives include salts and solvates. Salts which may be mentioned include acid addition salts. Pharmaceutically acceptable derivatives also include, at the bispidine nitrogens, C₁₋₄ alkyl quaternary ammonium salts and N-oxides, provided that when a N-oxide is present:

(a) no Het (Het¹, Het², Het³) group contains an unoxidised S-atom; and/or

(b) p does not represent 0 when B represents —(CH₂)ₙS(O)ₚ—.

The compounds of the invention may exhibit tautomerism. All tautomeric forms and mixtures thereof are included within the scope of the invention.

The compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of.the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric esters by conventional means (e.g. HPLC, chromatography over silica). All stereoisomers are included within the scope of the invention.

Alkyl groups that R¹, R², R^{2b}, R^{2c}, R^{2d}, R³, R⁴, R⁵, R⁶, R⁷, R⁸, R⁹, R¹⁰, R¹¹, R^{11a}, R¹², R¹³, R¹⁴, R¹⁵, R^{15a}, R¹⁶, R¹⁷, R¹⁸, R¹⁹, R²⁰, R²¹, R²², R⁴¹, R⁴², R⁴³, R⁴⁴, R⁴⁵ and R⁴⁶ may represent, that R¹² may include, and with which R³, R⁴, R⁷, R⁸ and R¹² may be substituted; and alkoxy groups that R⁴ may represent, and with which R⁴, R⁷, R⁸ and R¹² may be substituted; may be linear or, when there is a sufficient number (i.e. three) of carbon atoms, be branched and/or cyclic. Further, when there is a sufficient number (i.e. four) of carbon atoms, such alkyl and alkoxy groups may also be part cyclic/acyclic. Such alkyl and alkoxy groups may also be saturated or, when there is a sufficient number (i.e. two) of carbon atoms, be unsaturated and/or interrupted by oxygen.

Alkylene groups that R³ and R⁴, R⁸ and R⁹, R¹⁵ and R^{15a}, A, and B, may represent; and —(CH₂)ₘ—, —(CH₂)ₙ—, —(CH₂)_{q}— and —(CH₂)ᵣ— chains that A, B and R⁴ (as appropriate) may include, may be linear or, when there is a sufficient number (i.e. two) of carbon atoms, be branched. Such alkylene groups and —(CH₂)— containing chains may also be saturated or, when there is a sufficient number (i.e. two) of carbon atoms, be unsaturated and/or interrupted by oxygen.

Halo groups that R⁵ may represent, and with which R⁴, R⁷, R⁸ and R¹² may be substituted, include fluoro, chloro, bromo and iodo.

For the avoidance of doubt, each R¹⁰, R¹¹, and R^{11a}, group identified herein is independent of other R¹⁰, R¹¹, and R^{11a}, groups, respectively. For example, when R⁴ and R⁷ both represent aryl substituted by —C(O)R¹⁰, the two individual —C(O)R¹⁰ substituents are independent of one another, and are not necessarily identical (though this possibility is not excluded).

Abbreviations are listed at the end of this specification.

According to a further aspect of the invention there is provided compounds of formula I as hereinbefore defined, but with the further provisos that:

(a) when A represents —N(R¹⁶)(CH₂)ᵣ— or —O(CH₂)ᵣ—, then r does not represent 0 or 1; and

(b) when R⁵ represents —OH or —N(R¹³)R¹², then B does not represent —N(R¹⁷)C(O)O(CH₂)— or —N(R¹⁷)(CH₂)ₙ—.

Preferred compounds of the invention include those in which:

R¹ represents H;

R² represents H;

R³ represents

H;

C₁₋₂ alkyl; or,

together with R⁴ represents C₄₋₅ alkylene, optionally interrupted by an O atom and/or optionally substituted by one or more methyl groups;

R⁴ represents

H;

linear or branched and/or saturated or unsaturated and/or cyclic, acyclic and/or part cyclic/acyclic C₁₋₈ alkyl (which alkyl group is optionally substituted by one or more cyano or halo groups and/or interrupted by an O atom);

C₁₋₆ alkoxy;

—(CH₂)_{q}S(O)₂R⁸, (CH₂)_{q}C(O)OR⁸, (CH₂)_{q}N(H)C(O)R⁸, —(CH₂)_{q}C(O)R⁸, (in which latter four groups, q represents 0, 1 or 2 and R⁸ represents linear or branched and/or acyclic, cyclic and/or part cyclic/acyclic C₁₋₄ alkyl, or phenyl (which phenyl group is optionally substituted by one or more cyano and/or C₁₋₃ alkyl groups));

—(CH₂)_{q}C(O)N(R)R⁸ (in which latter group, q represents 0, 1 or 2 and R⁸ and R⁹ independently represent H, linear or branched and/or acyclic, cyclic and/or part cyclic/acyclic C₁₋₄ alkyl, or together represent C₄₋₆ allkylene);

—(CH₂)_{q}-phenyl, —(CH₂)_{q}-oxyphenyl or —(CH₂)_{q}-Het¹ (in which latter three groups, q represents 0, 1, 2 or 3, the —(CH₂)_{q}— part is optionally substituted by a cyano group, and the phenyl, or Het¹, part is optionally substituted with one or more substituents selected from cyano, nitro, linear or branched C₁₋₄ alkyl, linear or branched C₁₋₄ alkoxy and N(H)S(O)₂R^{11a}); or,

together with R³, represents C₄₋₅ alkylene, optionally interrupted by an O atom and/or optionally substituted by one or more methyl groups; R⁵ represents

H;

fluoro;

OR¹² (in which R¹² represents H, phenyl (optionally substituted by one or more methoxy groups) or C(O)N(H)R^{15a} (in which R^{15a} represents linear or branched C₁₋₄ alkyl));

—N(R¹³)(R¹²) (in which R¹² represents H, C₁₋₂ alkyl, —S(O)₂—C₁₋₂ alkyl, —C(O)R¹⁴ (in which R¹⁴ represents C₁₋₂ alkyl), —C(O)OR¹⁴ (in which R¹⁴ represents linear or branched C₁₋₅ alkyl) or —C(O)N(R¹⁵)(R^{15a}) (in which R¹⁵ and R^{15a} independently represent H or linear or branched C₁₋₃ alkyl or together represent C₄₋₅ alkylene, which alkylene group is optionally interrupted by O atom) and R¹³ represents H or C₁₋₂ alkyl); or,

together with R⁶, represents ═O (especially in the case where R⁷ represents alkyl or Het²);

R⁶ represents H or C₁₋₂ alkyl or together with R⁵ represents ═O (especially in the case where R⁷ represents alkyl or Het²);

A represents a single bond, linear or branched C₁₋₄ alkylene (which group is also optionally interrupted by O), —N(H)(CH₂)ᵣ— or —O(CH₂)ᵣ— (in which latter two groups r is 1 or 2);

B represents a single bond, C₁₋₄ alkylene, —(CH₂)ₙO—, —(CH₂)ₙS(O)₂—, —(CH₂)ₙN(H)— or —N(H)(CH₂)ₙ— (in which latter four cases n is 0, 1, 2 or 3);

R⁷ represents

linear or branched and/or acyclic, cyclic and/or part cyclic/acyclic C₁₋₆ alkyl (optionally substituted and/or terminated by OH);

Het² (optionally substituted by one or more substituents selected from cyano, C₁₋₃ alkyl, phenyl (which latter group is optionally substituted with one or more cyano groups), ═O, C(O)R¹⁰ (in which R¹⁰ is linear or branched C₁₋₃ alkyl) or S(O)₂R¹⁹ (in which R¹⁹ is C₁₋₂ alkyl)); or phenyl (optionally substituted by one or more substituents selected from cyano, nitro, linear or branched C₁₋₃ alkyl, linear or branched C₁₋₃ alkoxy, fluoro, chloro, C(O)N(H)R²² (in which R²² represents linear or branched and/or acyclic, cyclic and/or part cyclic/acyclic C₁₋₄ alky, which alkyl group is optionally terminated by cyano), N(H)S(O)₂R¹⁸ (in which R¹⁸ represents C₁₋₂ alkyl) or Het³);

R⁴¹, R⁴², R⁴³, R⁴⁴, R⁴⁵ and R⁴⁶ all represent H.

More preferred compounds of the invention include those in which:

R³ represents H;

R⁵ represents H, OH or —N(H)C(O)N(R¹⁵)(R^{15a});

R⁶ represents H;

A represents —CH₂— or —(CH₂)₂—;

B represents a single bond, —CH₂N(H)— or —CH₂O— (where, for the avoidance of doubt, the —CH₂— part is attached to the carbon atom bearing R⁵ and R⁶);

R⁷ represents phenyl (substituted by a cyano group (preferably in the 4-position relative to B) and by one or more optional C(O)N(H)R²² substituent).

Preferred compounds of the invention include the compounds of the Examples disclosed hereinafter.

### Preparation

According to the invention there is also provided a process for the preparation of compounds of formula I which comprises:

(a) for compounds of formula I in which R³ is H, reaction of a compound of formula II,

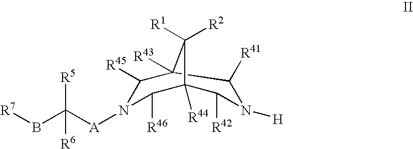

wherein R¹, R², R⁵, R⁶, R⁷, R⁴¹, R⁴², R⁴³, R⁴⁴, R⁴⁵, R⁴⁶, A and B are as hereinbefore defined with a compound of formula III,

R⁴—N═C═O III

wherein R⁴ is as hereinbefore defined, for example at between 0° C. and reflux temperature in the presence of an appropriate organic solvent (e.g. dichloromethane), or via solid phase synthesis under conditions known to those skilled in the art;

(b) reaction of a compound of formula II, as hereinbefore defined, with a carbonic acid derivative of formula IV,

(R³)(R⁴)NC(O)-L¹ IV

wherein L¹ represents a leaving group such as halo, imidazole or R²³O— (wherein R²³ represents, for example, C₁₋₁₀ alkyl, aryl or C₁₋₃ alkylaryl, which groups are optionally substituted by one or more halo or nitro groups) and R³ and R⁴ are as hereinbefore defined, for example at between room and reflux temperature in the presence of a suitable base (e.g. triethylamine or potassium carbonate) and an appropriate organic solvent (e.g. dichloromethane, THF, acetonitrile, toluene, or mixtures thereof);

(c) reaction of a compound of formula V,

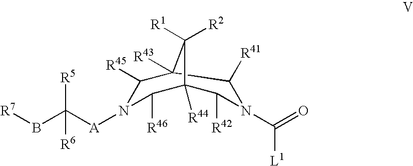

wherein R¹, R², R⁵, R⁶, R⁷, R⁴¹, R⁴², R⁴³, R⁴⁴, R⁴⁵, R⁴⁶, A, B and L¹ are as hereinbefore defined with a compound of formula VA,

(R³)(R⁴)NH VA

wherein R³ and R⁴ are as hereinbefore defined, for example at between room and reflux temperature in the presence of a suitable base (e.g. triethylamine or potassium carbonate) and an appropriate organic solvent (e.g. dichloromethane, THF, acetonitrile, toluene, or mixtures thereof), or via solid phase synthesis under conditions known to those skilled in the art;

(d) for compounds of formula I in which A represents CH₂ and R⁵ represents —OH or —N(H)R¹², wherein R¹² is as hereinbefore defined, reaction of a compound of formula VI,

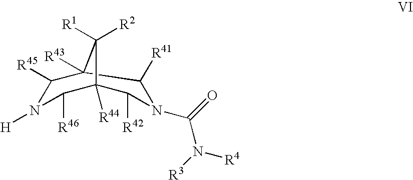

wherein R¹, R², R³, R⁴, R⁴¹, R⁴² R⁴³, R⁴⁴, R⁴⁵ and R⁴⁶ are as hereinbefore defined, with a compound of formula VII,

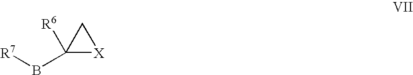

wherein X represents O or N(R¹²) and R⁶, R⁷, R¹² and B are as hereinbefore defined, for example at elevated temperature (e.g. 60° C. to reflux) in the presence of a suitable solvent (e.g. a lower alkyl alcohol (e.g. IPA), acetonitrile, or a mixture of a lower alkyl alcohol and water);

(e) reaction of a compound of formula VI, as hereinbefore defined, with a compound of formula VIII,

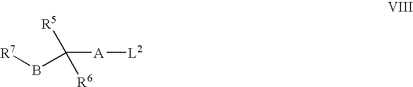

wherein L² represents a leaving group (e.g. mesylate, tosylate or halo) and R⁵, R⁶, R⁷, A and B are as hereinbefore defined, for example at elevated temperature (e.g. between 35° C. and reflux temperature) in the presence of a suitable base (e.g. triethylamine or K₂CO₃) and an appropriate organic solvent (e.g. acetonitrile or DMSO);

(f) for compounds of formula I in which R⁵ represents H or OH and R⁶ represents H, reduction of a compound of formula IX,

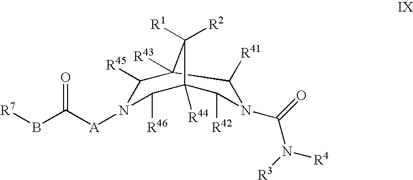

wherein R¹, R², R³, R⁴, R⁷, R⁴¹, R⁴², R⁴³, R⁴⁴, R⁴⁵, R⁴⁶, A and B are as hereinbefore defined, in the presence of a suitable reducing agent and under appropriate reaction conditions; for example, for formation of compounds of formula I in which R⁵ represents OH, reduction may be performed under mild reaction conditions in the presence of e.g. sodium borohydride and an appropriate organic solvent (e.g. THF); and for formation of compounds of formula I in which R⁵ represents H, reduction may be performed by activating the relevant C═O group using an appropriate agent (such as tosythydrazine) in the presence of a suitable reducing agent (e.g. sodium borohydride or sodium cyanoborohydride) and an appropriate organic solvent (e.g. a lower (e.g. C₁₋₆) alkyl alcohol);

(g) for compounds of formula I in which R¹ and R² both represent H, reduction of a corresponding compound of formula X,

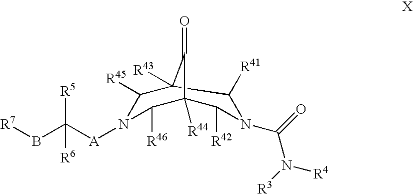

wherein R³, R⁴, R⁵, R⁶, R⁷, R⁴¹, R⁴², R⁴³, R⁴⁴, R⁴⁵, R⁴⁶, A and B are hereinbefore defined, and in which the bridgehead C═O group may be activated using an appropriate agent, such as tosylhydrazine, in the presence of a suitable reducing agent (e.g. sodium borohydride, sodium cyanoborohydride) and an appropriate organic solvent (e.g. a lower alkyl alcohol), or under standard Wolff-Kischner conditions known to those skilled in the art; when the C═O group is activated, the activation step may be carried out at between room and reflux temperature in the presence of an appropriate organic solvent (e.g. a lower alky alcohol such as methanol, ethanol or IPA), whereafter the reducing agent may be added to the reaction mixture and the reduction carried out at between 60° C. and reflux, advantageously in the presence of a suitable organic acid (e.g. acetic acid);

(h) for compounds of formula I in which R¹ and R² together represent —O(CH₂)₂O—, reaction of a corresponding compound of formula X as hereinbefore defined with ethane-1,2-diol under appropriate reaction conditions, for example by refluxing in the presence of pTSA and an appropriate organic solvent (e.g. toluene);

(i) for compounds of formula I in which B represents —(CH₂)ₙO—, reaction of a compound of formula XI,

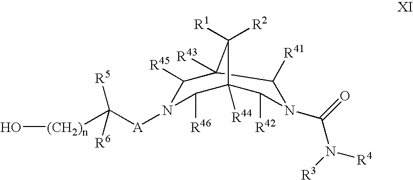

wherein R¹, R², R³, R⁴, R⁵, R⁶, R⁴, R⁴², R⁴³, R⁴⁵, R⁴⁶, A and n are as hereinbefore defined, with a compound of formula XIA,

R⁷OH XIA

in which R⁷ is as hereinbefore defined, for example under Mitsunobu-type conditions e.g. at between ambient (e.g. 25° C.) and reflux temperature in the presence of a tertiary phosphine (e.g. tributylphosphine or triphenylphosphine), an azodicarboxylate derivative (e.g. diethylazodicarboxylate or 1,1′-(azodicarbonyl)dipiperidine) and an appropriate organic solvent (e.g. dichloromethane or toluene);

(j) for compounds of formula I which are bispidine-nitrogen N-oxide derivatives, oxidation of the corresponding bispidine nitrogen of a corresponding compound of formula I, in the presence of a suitable oxidising agent (e.g. mCPBA), for example at 0° C. in the presence of a suitable organic solvent (e.g. DCM);

(k) for compounds of formula I which are C₁₋₄ alkyl quaternary ammonium salt derivatives, in which the alkyl group is attached to a bispidine nitrogen, reaction, at the bispidine nitrogen, of a corresponding compound of formula I with a compound of formula XII,

R^{b}L³ XII

wherein R^{b} represents C₁₋₄ alkyl and L³ is a leaving group such as halo, alkane sulfonate or aryl sulfonate, for example at room temperature in the presence of an appropriate organic solvent (e.g. DMF), followed by purification (using e.g. HPLC) in the presence of a suitable counter-ion provider (e.g. NH₄OAc);

(l) for compounds of formula I in which R⁵ and R⁶ represent H, A represents C₁₋₆ alkylene and B represents —N(R¹⁷)(CH₂)ₙ—, reaction of a compound of formula XIII,

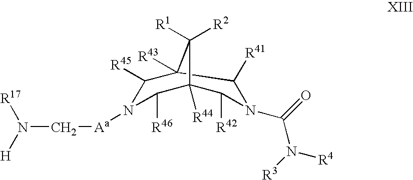

wherein A^{a} represents C₁₋₆ alkylene and R¹, R², R³, R⁴, R⁴¹, R⁴², R⁴³, R⁴⁴, R⁴⁵, R⁴⁶ and R¹⁷ are as hereinbefore defined with a compound of formula XIV,

R⁷—(CH₂)ₙ-L² XIV

wherein R⁷, n and L² are as hereinbefore defined, for example at 40° C. in the presence of a suitable organic solvent (e.g. acetonitrile);

(m) for compounds of formula I in which R⁵ represents —NH₂, reduction of a corresponding compound of formula XV,

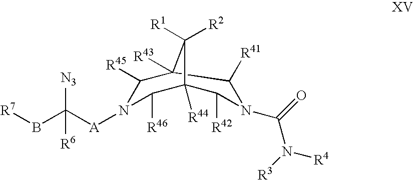

wherein R¹, R², R³, R⁴, R⁶, R⁷, R⁴¹, R⁴², R⁴³, R⁴⁴, R⁴⁵, R⁴⁶, A and B are as hereinbefore defined, for example by hydrogenation at a suitable pressure in the presence of a suitable catalyst (e.g. palladium on carbon) and an appropriate solvent (e.g. a water-ethanol mixture);

(n) for compounds of formula I in which R⁵ represents —N(R¹³)C(O)NH(R¹⁵), reaction of a corresponding compound of formula I in which R⁵ represents —N(R¹³)H with a compound of formula XVI,

R¹⁵N═C═O XVI

wherein R¹⁵ is as hereinbefore defined, for example at ambient temperature (e.g. 25° C.) in the presence of a suitable solvent (e.g. benzene);

(o) for compounds of formula I in which R⁵ represents —N(R¹³)C(O)R¹⁴, reaction of a corresponding compound of formula I in which R⁵ represents —N(R¹³)H with a compound of formula XVII,

R¹⁴C(O)R^{x} XVII

wherein R^{x} represents a suitable leaving group, such as C₁₋₄ alkoxy, halo (e.g. Cl, Br) or p-nitrophenyl, and R¹⁴ is as hereinbefore defined, for example at between ambient and reflux temperature in the presence of a suitable solvent (e.g. dichloromethane or acetonitrile) and optionally in the presence of a suitable base (e.g. triethylamine or potassium carbonate);

(p) for compounds of formula I in which R⁵ represents —N(H)R¹², wherein R¹² is as previously defined provided that it does not represent H, reaction of a corresponding compound of formula I, in which R⁵ represents —NH₂ with a compound of formula XVIII,

R^{12a}L¹ XVIII

wherein R^{12a} represents R¹² as hereinbefore defined except that it does not represent H and L¹ is as hereinbefore defined, for example under conditions that are well known to those skilled in the art;

(q) for compounds of formula I in which R⁵ represents —OR¹² in which R¹² represents C₁₋₆ alkyl or optionally substituted aryl, reaction of a corresponding compound of formula I in which R⁵ represents —OH with a compound of formula XIX,

R^{12a}OH XIX

wherein R^{12a} represents C₁₋₆ alkyl or optionally substituted aryl, for example at between ambient (e.g. 25° C.) and reflux temperature, under Mitsunobu-type conditions (i.e. in the presence of e.g. triphenylphosphine, an azodicarboxylate derivative (e.g. 1,1′-(azodicarbonyl)dipiperidine) and a suitable organic solvent (e.g. dichloromethane));

(r) for compounds of formula I in which R⁵ represents —OR², in which R¹² represents C₁₋₆ alkyl or optionally substituted aryl, reaction of a compound of formula XX,

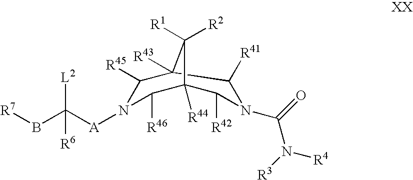

wherein L², R¹, R², R³, R⁴, R⁶, R⁷, R⁴¹, R⁴², R⁴³, R⁴⁴, R⁴⁵, R⁴⁶, A and B are as hereinbefore defined with a compound of formula XIX as hereinbefore defined, for example at between ambient (e.g. 25° C.) and reflux temperature, under Williamson-type conditions (i.e. in the presence of an appropriate base (e.g. KOH or NaH) and a suitable organic solvent (e.g. dimethylsulfoxide or DMF));

(s) for compounds of formula I in which R⁵ represents OR¹² and R¹² represents C(O)R¹⁴ and R¹⁴ is as hereinbefore defined, reaction of a corresponding compound of formula I as hereinbefore defined in which R⁵ represents OH with a compound of formula XXI,

R¹⁴CO₂H XXI

wherein R¹⁴ is as hereinbefore defined, for example at ambient temperature (e.g. 25° C.) in the presence of a suitable coupling agent (e.g. 1-(3-dimethylaminopropyl)-3-ethylcarbodiimide), an appropriate catalyst (e.g. 4-dimethylaminopyridine) and a reaction-inert organic solvent (e.g. THF);

(t) for compounds of formula I in which R⁵ represents halo, substitution of a corresponding compound of formula I in which R⁵ represents —OH, using an appropriate halogenating agent (e.g., for compounds in which R⁵ represents fluoro, reaction with diethylaminosulfurtrifluoride);

(u) for compounds of formula I in which R³ and/or R⁴ as appropriate represent alkyl groups (e.g. C₁₋₆ or C₁₋₁₂ alkyl, as appropriate), alkylation of a corresponding compound of formula I, in which R³ and/or R⁴ (as appropriate) represent H under conditions well known to those skilled in the art;

(v) conversion of one R⁴ group to another (e.g. conversion of —(CH₂)_{q}C(O)OR⁸ to —(CH₂)_{q}C(O)N(R⁹)R⁸, wherein R⁸, R⁹ and q are as hereinbefore defined) using techniques well known to those skilled in the art; or

(w) for compounds of formula I in which one of R¹ and R² represents H, and the other represents —OH, reduction of a corresponding compound of formula X, as hereinbefore defined, in the presence of a mild reducing agent, e.g. sodium borohydride, and an appropriate organic solvent (e.g. a lower alcohol such as methanol or ethanol);

(x) for compounds of formula I in which one of R² and R³ represents —NH₂ and the other represents H, reduction of a compound of formula XXIA,

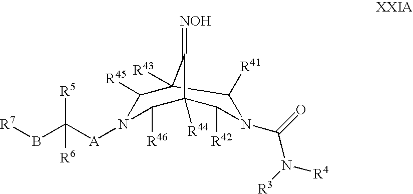

wherein R³, R⁴, R⁵, R⁶, R⁷, R⁴¹, R⁴², R⁴³, R⁴⁴, R⁴⁵, R⁴⁶, A and B are as hereinbefore defined, in the presence of a suitable reducing agent (e.g. LiAlH₄), for example under conditions that are well known to those skilled in the art;

(y) for compounds of formula I in which one or both of R¹ and R² represent —N(R^{2c})R^{2d} in which one or both of R^{2c} and R^{2d} represents C₁₋₆ alkyl, alkylation of a corresponding compound of formula I in which R¹ and/or R² represent —N(R^{2c})R^{2d} (as appropriate) in which R^{2c} and/or R^{2d} (as appropriate) represent H, using a compound of formula XXIB,

R^{2e}L¹ XXIB

wherein R^{2e} represents C₁₋₆ alkyl and L¹ is as hereinbefore defined, for example under conditions that are well known to those skilled in the art; or

(z) conversion of one substituent on R⁷ to another using techniques well known to those skilled in the art.

Compounds of formula II may be prepared by reaction of a compound of formula XXII,

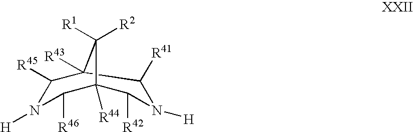

wherein R¹, R², R⁴¹, R⁴², R⁴³, R⁴⁴, R⁴⁵ and R⁴⁶ are as hereinbefore defined, with a compound of formula VIII as hereinbefore defined, for example as described hereinbefore for synthesis of compounds of formula I (process step (e)), or, in the case of compounds of formula II wherein A represents CH₂ and R⁵ represents OH or N(H)R¹², with a compound of formula VII as hereinbefore defined, for example as described hereinbefore for synthesis of compounds of formula I (process step (d)).

Compounds of formula II in which R¹ and R² both represent H may be prepared by reduction of a compound of formula XXIII,

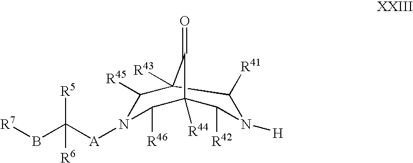

wherein R⁵, R⁶, R⁷, R⁴¹, R⁴², R⁴³, R⁴⁴, R⁴⁵, R⁴⁶, A and B are as hereinbe fore defined, and in which the C═O group may be activated using an appropriate agent, such as tosylhydrazine, for example as described hereinbefore for synthesis of compounds of formula I (process step (g)).

Compounds of formula IV may be prepared by reaction of a compound of formula VA, as hereinbefore defined, with a compound of formula XXIV,

L¹-C(O)-L¹ XXIV

wherein L¹ is as hereinbefore defined, and in which the two L¹ groups may be the same or different, for example at between 0° C. and reflux temperature in the presence of a suitable base (e.g. triethylamine or potassium carbonate) and an appropriate organic solvent (e.g. toluene or dichloromethane).

Compounds of formula V may be prepared by reaction of a compound of formula II, as hereinbefore defined, with a compound of formula XXIV, as hereinbefore defined, for example as described hereinbefore for the synthesis of compounds of formula IV.

Compounds of formula VI may be prepared by reaction of a compound of formula XXII, as hereinbefore defined, with a compound of formula II, as hereinbefore defined, for example as described hereinbefore for synthesis of compounds of formula I (process step (a)), or with a compound of formula IV, as hereinbefore defined, for example as described hereinbefore for synthesis of compounds of formula I (process step (b)).

Compounds of formula VI may alternatively be prepared by reaction of a compound of formula XXII, as hereinbefore defined, with a compound of formula XXIV, as hereinbefore defined, for example as described hereinbefore for synthesis of compounds of formula IV, followed by reaction of the resultant intermediate with a compound of formula VA, as hereinbefore defined, for example as described hereinbefore for the synthesis of compounds of formula I (process step (c)).

Compounds of formula VI in which R¹ and R² represent H may alternatively be prepared by reduction of a corresponding compound of formula XXV,

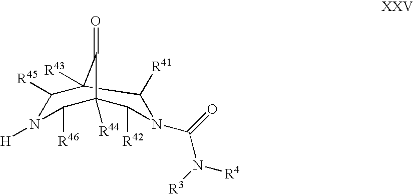

wherein R³, R⁴, R⁴¹, R⁴², R⁴³, R⁴⁴, R⁴⁵ and R⁴⁶ are as hereinbefore defined, and in which the C═O group may be activated using an appropriate agent, such as tosylhydrazine, for example as described hereinbefore for compounds of formula I (process step (g)).

Compounds of formula VI in which one or more of R⁴¹, R⁴², R⁴⁵ and/or R⁴⁶ represent C₁₋₃ alkyl may be prepared by reaction of a compound of formula VI in which R⁴¹, R⁴², R⁴⁵ and/or R⁴⁶ (as appropriate) represent H, with an appropriate alkylating agent (e.g. dimethyl sulfate), for example in the presence of a suitable strong base (e.g. s-BuLi), N,N,N′,N′-tetramethylethylenediamine and a reaction-inert solvent (e.g. THF).

Compounds of formula VII may be prepared in accordance with techniques which are known to those skilled in the art. For example, compounds of formula VII in which:

(1) B represents —CH₂O— and X represents O may be prepared by reaction of a compound of formula XIA as hereinbefore defined, with a compound of formula XXVI,

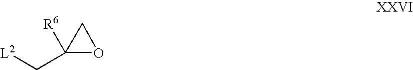

wherein R⁶ and L² are as hereinbefore defined, for example at elevated temperature (e.g. between 60° C. and reflux temperature) in the presence of a suitable base (e.g. K₂CO₃ or NaOH) and an appropriate organic solvent (e.g. acetonitrile or toluene/water), or as otherwise described in the prior art;

(2) R⁶ represents H and X represents O may be prepared by reduction of a compound of formula XXVII,

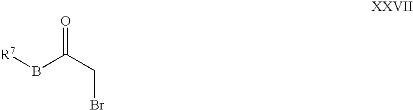

wherein R⁷ and B are as hereinbefore defined, for example at between −15° C. and room temperature in the presence of a suitable reducing agent (e.g. NaBH₄) and an appropriate organic solvent (e.g. THF), followed by an internal displacement reaction in the resultant intermediate, for example at room temperature in the presence of a suitable base (e.g. K₂CO₃) and an appropriate organic solvent (e.g. acetonitrile);

(3) B represents C₁₋₄ alkylene, —(CH₂)ₙN(R¹⁷)—, —(CH₂)ₙS(O)₂— or —(CH₂)ₙO— (in which latter three groups n represents 1, 2, 3 or 4) or —(CH₂)ₘC(H)(OH)(CH₂)ₙ— and X represents O may be prepared by oxidation of a compound of formula XXVIII,

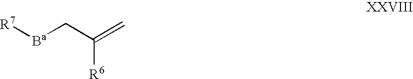

in which B^{a} represents a single bond, C₁₋₃ alkylene, —(CH₂)ₙ₋₁N(R¹⁷)—, —(CH₂)ₙ₋₁S(O)₂— or —(CH₂)ₙ₋₁O —(in which latter three groups n represents 1, 2, 3 or 4) or —(CH₂)ₘ₋₁C(H)(OH)(CH₂)ₙ— (in which latter group n is as hereinbefore defined), and in all cases R¹⁷ and m are as hereinbefore defined, in the presence of a suitable oxidising agent (e.g. mCPBA), for example by refluxing in the presence of a suitable organic solvent (e.g. DCM); or

(4) B represents —(CH₂)ₙO— and X represents N(R¹²) and R¹² represents —S(O)₂—C₁₋₄-alkyl or —C(O)OR¹⁴ may be prepared by cyclisation of a compound of formula XXVIIIA,

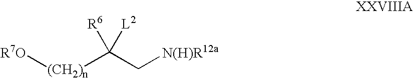

wherein R^{12a} represents —S(O)₂—C₁₋₄-alkyl or —C(O)OR¹⁴ and n, R⁶, R⁷, R¹⁴ and L² are as hereinbefore defined, for example at between 0° C. and reflux temperature in the presence of a suitable base (e.g. sodium hydroxide), an appropriate solvent (e.g. dichloromethane, water, or a mixture thereof) and, if necessary a phase transfer catalyst (such as tetrabutylammonium hydrogensulfate).

Compounds of formula VIII may be prepared by standard techniques. For example compounds of formula VIII in which:

(1) B represents —(CH₂)ₙO— may be prepared by coupling a compound of formula XIA, as hereinbefore defined, to a compound of formula XXIX,

L⁴-(CH₂)ₙ—C(R⁵)(R⁶)-A-L² XXIX

wherein L⁴ represents a suitable leaving group (e.g. halo) and n, R⁵, R⁶, A and L² are as hereinbefore defined; or

(2) B represents —C(O)N(R¹⁷)— may be prepared by coupling a compound of formula XXX,

R⁷N(H)R¹⁷ XXX

wherein R⁷ and R¹⁷ are as hereinbefore defined, to a compound of formula XXXI,

L⁴-C(O)—C(R⁵)(R⁶)-A-L² XXXI

wherein L⁴, R⁵, R⁶, A and L² are as hereinbefore defined;

in both cases, under conditions which are well known to those skilled in the art.

Compounds of formula VIII in which A represents C₂-alkylene and R⁵ represents OR¹², in which R¹² represents C₁₋₆ alyl or optionally substituted aryl may alternatively be prepared by reaction of a compound of formula XIX as hereinbefore defined with a compound of formula XXXIA,

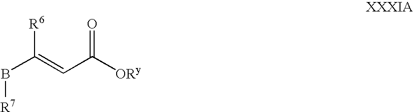

wherein R^{y} represents C₁₋₄ alkyl or aryl (which two groups are optionally substituted with one or more substituents selected from C₁₋₄ alkyl or halo) and R⁶, R⁷ and B are as hereinbefore defined, for example at between ambient temperature (e.g. 25° C.) and reflux temperature in the presence of a suitable base (e.g. K₂CO₃) and an appropriate organic solvent (e.g. acetonitrile), followed by conversion of the ester functionality to an L² group (in which L² is as hereinbefore defined), under conditions that are well known to those skilled in the art.

Compounds of formulae VII and VIII in which B represents —(CH₂)ₙS(O)— or —(CH₂)ₙS(O)₂— may be prepared by oxidation of corresponding compounds of formulae VII and VIII wherein B represents —(CH₂)ₙS—, wherein n is as hereinbefore defined, in the presence of an appropriate amount of a suitable oxidising agent (e.g. mCPBA) and an appropriate organic solvent.

Compounds of formulae IX and XI may be prepared in a similar fashion to compounds of formula I (see, for example, process steps (a), (b), (c) or (d)).

Alternatively, compounds of formula IX in which A represents C₂ alkylene may be prepared by reaction of a compound of formula VI, as hereinbefore defined with a compound of formula XXXII,

R⁷—B—C(O)—CH═CH₂ XXXII

wherein B and R⁷ are as hereinbefore defined, for example a room temperature in the presence of a suitable organic solvent (e.g. ethanol).

Compounds of formula XIII may be prepared by removing an optionally substituted benzyloxycarbonyl unit from (i.e. deprotecting) a corresponding compound of formula I in which R⁷ represents optionally substituted phenyl, R⁵ and R⁶ both represent H, B represents —N(R¹⁷)C(O)O(CH₂)—, A represents A^{a} and A^{a} is as hereinbefore defined under conditions which are well known to those skilled in the art.

Compounds of formula XV may be prepared by reaction of a corresponding compound of formula I, as hereinbefore defined, in which R⁵ represents —OH, with a compound of formula XXXIII

R^{y}S(O)₂Cl XXXIII

wherein R^{y} is as hereinbefore defined, for example at between −10 and 25° C. in the presence of a suitable solvent (e.g. dichloromethane), followed by reaction with a suitable source of the azide ion (e.g. sodium azide) for example at between ambient and reflux temperature in the presence of an appropriate solvent (e.g. DMF) and a suitable base (e.g. NaHCO₃).

Compounds of formula XV may alternatively be prepared by reaction of a corresponding compound of formula VI, as hereinbefore defined with a compound of formula XXXIIIA,

R⁷—B—C(R⁶)(N₃)-A-L² XXXIIIA

wherein L², R⁶, R⁷, A and B are as hereinbefore defined, for example under analogous conditions to those described hereinbefore for preparation of compounds of formula I (process step (e)).

Compounds of formula XX may be prepared by replacement of the OH group of a compound of formula I in which R⁵ represents OH with an L² group under conditions that are well known to those skilled in the art.

Compounds of formula XXIA may be prepared by reaction of a corresponding compound of formula X with hydroxylamine, for example at elevated temperature (e.g. at reflux) in the presence of a suitable organic solvent (e.g. methanol).

Compounds of formula XXII are known in the literature or are readily available using known techniques. For example, compounds of formula XXII in which R¹ and R² together represent —O—(CH₂)₂—O—, —(CH₂)₃—, —(CH₂)₄— or —(CH₂)₅—, and R⁴¹, R⁴², R⁴³, R⁴⁴, R⁴⁵ and R⁴ all represent H, may be prepared by reduction of a compound of formula XXXIV,

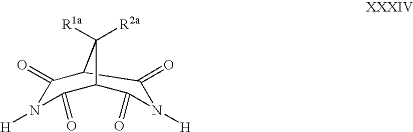

wherein R^{1a} and R^{2a} together represent —O—(CH₂)₂—O—, —(CH₂)₃—, —(CH₂)₄— or —(CH₂)₅—, in the presence of a suitable reducing agent (e.g. LiAlH₄) under conditions which are well known to those skilled in the art.

Compounds of formula XXXIIIA may be prepared in analogous fashion to compounds of formula XV (i.e. from the corresponding alcohol).

Compounds of formulae X, XXIII and XXV (in which, in all cases, R⁴⁵ and R⁴⁶ both represent H), may be prepared, advantageously, by reaction of (as appropriate) either (i) a compound of formula XXXV,

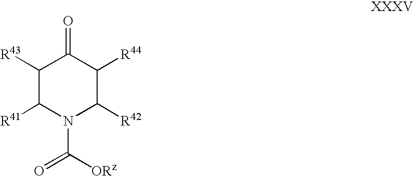

wherein R^{z} represents C₁₋₁₀ alkyl or C₁₋₃ alkylaryl (e.g. alkylphenyl, such as benzyl) and R⁴¹, R⁴², R⁴³ and R⁴⁴ are as hereinbefore defined, or (ii) 4-piperidone (or a protected derivative thereof), with (as appropriate) either (1) a compound of formula XXXVI,

R⁷—B—C(R⁵)(R⁶)-A-NH₂ XXXVI

wherein R⁵, R⁶, R⁷, A and B are as hereinbefore defined, or (2) NH₃ (or a protected (e.g. benzyl) derivative thereof), in all cases in the presence of a formaldehyde (i.e. an appropriate source of formaldehyde, such as paraformaldehyde or formalin solution) and, in the case of compounds of formulae X and XXV, conversion of the C(O)OR^{z} group in the resultant intermediate to a C(O)N(R³)(R⁴) group using techniques such as those described herein (e.g. process step (c) above).

The formation of compounds of formulae X, XXIII and XXV may be carried out in this way for example at between room temperature and reflux (depending upon the concentration of the reactants) in the presence of an appropriate solvent (e.g. ethanol or methanol) and, preferably, in the presence of an organic acid (e.g. a C₁₋₆ carboxylic acid, especially acetic acid).

It will be also appreciated by those skilled in the art that compounds of formula XXII in which R¹ and R² both represent H may also be prepared via this method (i.e. by reaction of a compound of 4-piperidone (or a protected derivative thereof) with NH₃ (or a protected derivative thereof) in the presence of a formaldehyde), provided that the intermediate so formed is subsequently reduced under appropriate reaction conditions.

The skilled person will also appreciate that this process may also be used to prepare compounds of formula I in which R⁴¹ and R⁴² are H, and R⁴⁵ and/or R⁴⁶ are other than H, for example by:

(i) reacting a compound of formula XXXV in which R⁴¹ and/or R⁴² is/are other than H with, for example, benzylamine or a derivative thereof;

(ii) removal of the —C(O)OR^{z} unit;

(iii) reaction at the free bispidine nitrogen of the resultant compound with a compound of formula VIII as hereinbefore defined;

(iv) removal of the benzyl protecting group; and

(v) reaction at the free bispidine nitrogen of the resultant compound with, for example, a compound of formula III or IV as hereinbefore defined,

under conditions well known to those skilled in the art including those described hereinbefore. This reaction will be accompanied by, at some point, conversion of the bridgehead carbonyl functionality to the desired R¹/R² groups.

Compounds of formula XXXIV may be prepared in accordance with techniques which are well known to those skilled in the art. For example, compounds of formula XXXIV in which R^{1a} and R^{2a} together represent —(CH₂)₃—, —(CH₂)₄— or —(CH₂)₅— may be prepared by reaction of a compound of formula XXXVII,

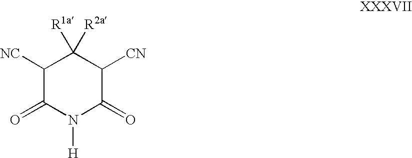

wherein R^{1a′} and R^{2′} together represent —(CH₂)₃—, —(CH₂)₄— or —(CH₂)₅—, with a mixture of phosphoric acid and sulfuric acid, for example at 120° C.

Compounds of formula XXXVI are well known in the literature or are readily available using known techniques. For example, compounds of formula XXXVI wherein R⁵ represents OH, R⁶ represents H and A represents CH₂ may be prepared by reaction of a compound of formula VII in which R⁶ represents H and X represents O with ammonium hydroxide under conditions which are well known to those skilled in the art.

Compounds of formulae III, VA, XIA, XII, XIV, XVI, XVII, XVIII, XIX, XXI, XXIB, XXIV, XXVI, XXVII, XXVIII, XXVIIIA, XXIX, XXX, XXXI, XXXIA, XXXII, XXXIII, XXXV and XXXVII and derivatives thereof, are either commercially available, are known in the literature, or may be obtained either by analogy with the processes described herein, or by conventional synthetic procedures, in accordance with standard techniques, from readily available starting materials using appropriate reagents and reaction conditions.

Substituents on the aryl (e.g. phenyl), and (if appropriate) heterocyclic, group(s) in compounds defined herein may be converted to other claimed substituents using techniques well known to those skilled in the art. For example, nitrobenzene may be reduced to an aminobenzene, hydroxy may be converted to alkoxy, alkoxy may be hydrolysed to hydroxy, etc.

The compounds of the invention may be isolated from their reaction mixtures using conventional techniques.

It will be appreciated by those skilled in the art that, in the process described above, the functional groups of intermediate compounds may be, or may need to be, protected by protecting groups.

Functional groups which it is desirable to protect include hydroxy, amino and carboxylic acid. Suitable protecting groups for hydroxy include trialkylsilyl and diarylalkylsilyl groups (e.g. tert-butyldimethylsilyl, tert-butyldiphenylsilyl or trimethylsilyl), tetrahydropyranyl and alkylcarbonyloxy groups (e.g. methyl- and ethylcarbonyloxy groups). Suitable protecting groups for amino include benzyl, tert-butyloxycarbonyl, 9-fluorenylmethoxycarbonyl or benzyloxycarbonyl. Suitable protecting groups for carboxylic acid include C₁₋₆ alkyl or benzyl esters.

The protection and deprotection of functional groups may take place before or after any of the reaction steps described hereinbefore.

Protecting groups may be removed in accordance with techniques which are well known to those skilled in the art and as described hereinafter.

The use of protecting groups is fully described in “Protective Groups in Organic Chemistry”, edited by J W F McOmie, Plenum Press (1973), and “Protective Groups in Organic Synthesis”, 2nd edition, T W Greene & P G M Wutz, Wiley-Interscience (1991).

Persons skilled in the art will appreciate that, in order to obtain compounds of the invention in an alternative, and, on some occasions, more convenient, manner, the individual process steps mentioned herein may be performed in a different order, and/or the individual reactions may be performed at a different stage in the overall route (i.e. substituents may be added to and/or chemical transformations performed upon, different intermediates to those associated hereinbefore with a particular reaction). This will depend inter alia on factors such as the nature of other functional groups present in a particular substrate, the availability of key intermediates and the protecting group strategy (if any) to be adopted. Clearly, the type of chemistry involved will influence the choice of reagent that is used in the said synthetic steps, the need, and type, of protecting groups that are employed, and the sequence for accomplishing the synthesis.

It will also be appreciated by those skilled in the art that, although certain protected derivatives of compounds of formula I, which may be made prior to a final deprotection stage, may not possess pharmacological activity as such, they may be administered parenterally or orally and thereafter metabolised in the body to form compounds of the invention which are pharmacologically active. Such derivatives may therefore be described as “prodrugs”. Moreover, we have found that certain compounds of formula I may act as prodrugs of other compounds of formula I.

All prodrugs of compounds of formula I are included within the scope of the invention.

Some of the intermediates referred to hereinbefore are novel. According to a further aspect of the invention there is thus provided: (a) a compound of formula II, as hereinbefore defined or a protected derivative thereof, provided that R⁷ does not represent optionally substituted phenyl; (b) a compound of formula V, as hereinbefore defined or a protected derivative thereof, provided that R⁷ does not represent optionally substituted phenyl; (c) a compound of formula X as hereinbefore defined or a protected derivative thereof; (d) a compound of formula XI as hereinbefore defined or a protected derivative thereof; (e) a compound of formula XIII, as hereinbefore defined or a protected derivative thereof; (f) a compound of formula XV, as hereinbefore defined or a protected derivative thereof; (g) a compound of formula XX, as hereinbefore defined or a protected derivative thereof; (h) a compound of formula XXIII, as hereinbefore defined or a protected derivative thereof, provided that R⁷ does not represent optionally substituted phenyl; and (i) a compound of formula XXV, as hereinbefore defined or a protected derivative thereof.

### Medical and Pharmaceutical Use

The compounds of the invention are useful because they possess pharmacological activity. They are therefore indicated as pharmaceuticals.

Thus, according to a further aspect of the invention there is provided the compounds of the invention for use as pharmaceuticals.

In particular, the compounds of the invention exhibit myocardial electrophysiological activity, for example as demonstrated in the test described below.

The compounds of the invention are thus expected to be useful in both the prophylaxis and the treatment of arrhythmias, and in particular atrial and ventricular arrhythmias.

The compounds of the invention are thus indicated in the treatment or prophylaxis of cardiac diseases, or in indications related to cardiac diseases, in which arrhythmias are believed to play a major role, including ischaemic heart disease, sudden heart attack, myocardial infarction, heart failure, cardiac surgery and thromboembolic events.

In the treatment of arrhythmias, compounds of the invention have been found to selectively delay cardiac repolarization, thus prolonging the QT interval, and, in particular, to exhibit class III activity. Although compounds of the invention have been found to exhibit class III activity in particular, in the treatment of arrhythmias, their mode(s) of activity is/are not necessarily restricted to this class.

According to a further aspect of the invention, there is provided a method of treatment of an arrhythmia which method comprises administration of a therapeutically effective amount of a compound of the invention to a person suffering from, or susceptible to, such a condition.

### Pharmaceutical Preparations

he compounds of the invention will normally be administered orally, subcutaneously, intravenously, intraarterially, transdermally, intranasally, by inhalation, or by any other parenteral route, in the form of pharmaceutical preparations comprising the active ingredient either as a free base, a pharmaceutically acceptable ion exchanger or a non-toxic organic or inorganic acid addition salt, in a pharmaceutically acceptable dosage form. Depending upon the disorder and patient to be treated, as well as the route of administration, the compositions may be administered at varying doses.

The compounds of the invention may also be combined with any other drugs useful in the treatment of arrhythmias and/or other cardiovascular disorders.

According to a further aspect of the invention there is thus provided a pharmaceutical formulation including a compound of the invention in admixture with a pharmaceutically acceptable adjuvant, diluent or carrier.

Suitable daily doses of the compounds of the invention in therapeutic treatment of humans are about 0.05 to 5.0 mg/kg body weight at parenteral administration.

The compounds of the invention have the advantage that they are effective against cardiac arrhythmias.

Compounds of the invention may also have the advantage that they may be more efficacious than, be less toxic than, have a broader range of activity (including exhibiting any combination of class I, class II, class III and/or class IV activity (especially class I, class II and/or class IV activity in addition to class III activity)) than, be more potent than, be longer acting than, produce fewer side effects (including a lower incidence of proarrhythmias such as torsades de pointes) than, be more easily absorbed than, or that they may have other useful pharmacological properties over, compounds known in the prior art.

### Biological Tests

Test A

Primary Electrophysiological Effects in Anaesthetised Guinea Pigs

Guinea pigs weighing between 660 an 1100 g were used. The animals were housed for at least one week before the experiment and had free access to food and tap water during that period.

Anaesthesia was induced by an intraperitoneal injection of pentobarbital (40 to 50 mg/kg) and catheters were introduced into one carotid artery (for blood pressure recording and blood sampling) and into one jugular vein (for drug infusions). Needle electrodes were placed on the limbs for recording of ECGs (lead II). A thermistor was placed in the rectum and the animal was placed on a heating pad, set to a rectal temperature of between 37.5 and 38.5° C.

A tracheotomy was performed and the animal was artificially ventilated with room air by use of a small animal ventilator, set to keep blood gases within the normal range for the species. In order to reduce autonomic influences both vagi were cut in the neck, and 0.5 mg/kg of propranolol was given intravenously, 15 minutes before the start of the experiment.

The left ventricular epicardium was exposed by a left-sided thoracotomy, and a custom-designed suction electrode for recording of the monophasic action potential (MAP) was applied to the left ventricular free wall. The electrode was kept in position as long as an acceptable signal could be recorded, otherwise it was moved to a new position. A bipolar electrode for pacing was clipped to the left atrium. Pacing (2 ms duration, twice the diastolic threshold) was performed with a custom-made constant current stimulator. The heart was paced at a frequency just above the normal sinus rate during 1 minute every fifth minute throughout the study.

The blood pressure, the MAP signal and the lead II ECG were recorded on a Mingograph ink-jet recorder (Siemens-Elema, Sweden). All signals were collected (sampling frequency 1000 Hz) on a PC during the last 10 seconds of each pacing sequence and the last 10 seconds of the following minute of sinus rhythm. The signals were processed using a custom-made program developed for acquisition and analysis of physiological signals measured in experimental animals (see Axenborg and Hirsch, Comput. Methods Programs Biomed. 41, 55 (1993)).

The test procedure consisted of taking two basal control recordings, 5 minutes apart, during both pacing and sinus rhythm. After the second control recording, the first dose of the test substance was infused in a volume of 0.2 mL into the jugular vein catheter for 30 seconds. Three minutes later, pacing was started and a new recording was made. Five minutes after the previous dose, the next dose of test substance was administered. Six to ten consecutive doses were given during each experiment.

Data Analysis

Of the numerous variables measured in this analysis, three were selected as the most important for comparison and selection of active compounds. The three variables selected were the MAP duration at 75 percent repolarization during pacing, the atrio-ventricular (AV) conduction time (defined as the interval between the atrial pace pulse and the start of the ventricular MAP) during pacing, and the heart rate (defined as the RR interval during sinus rhythm). Systolic and diastolic blood pressure were measured in order to judge the haemodynamic status of the anaesthetised animal. Further, the ECG was checked for arrhythmias and/or morphological changes.

The mean of the two control recordings was set to zero and the effects recorded after consecutive doses of test substance were expressed as percentage changes from this value. By plotting these percentage values against the cumulative dose administered before each recording, it was possible to construct dose-response curves. In this way, each experiment generated three dose-response curves, one for MAP duration, one for AV-conduction time and one for the sinus frequency (RR interval). A mean curve of all experiments performed with a test substance was calculated, and potency values were derived from the mean curve. All dose-response curves in these experiments were constructed by linear connection of the data points obtained. The cumulative dose prolonging the MAP duration by 10% from the baseline was used as an index to assess the class III electrophysiological potency of the agent under investigation (D₁₀).

Test B

Metabolic Stability of Test Compounds

An in vitro screen was set up to determine the metabolic stability of the compounds of the invention.

The hepatic S-9 fraction from dog, man, rabbit and rat with NADPH as co-factor was used. The assay conditions were as follows: S-9 (3 mg/mL), NADPH (0.83 mM), Tris-HCl buffer (50 mM) at pH 7.4 and 10 μM of test compound.

The reaction was started by addition of test compound and terminated after 0, 1, 5, 15 and 30 minutes by raising the pH in the sample to above 10 (NaOH; 1 mM). After solvent extraction, the concentration of test compound was measured against an internal standard by LC (fluorescence/UV detection).

The percentage of test compound remaining after 30 minutes (and thus t_{1/2}) were calculated and used as a measure for metabolic stability.

The invention is illustrated by way of the following examples.

### EXAMPLES

### General Experimental Procedures

Mass spectra were recorded on a Finnigan MAT TSQ 700 triple quadrupole mass spectrometer equipped with an electrospray interface (FAB-MS) and VG Platform II mass spectrometer equipped with an electrospray interface (LC-MS), a Hewlett Packard model 6890 gas chromatograph connected to a Hewlett-Packard model 5973A mass spectrometer via a Hewlett Packard HP-5-MS GC column, or a Shimadzu QP-5000 GC/mass spectrometer (CI, methane). ¹H NMR and ¹³C NMR measurements were performed on a BRUKER ACP 300 and Varian UNITY plus 400 and 500 spectrometers, operating at ¹H frequencies of 300, 400 and 500 MHz respectively, and at ¹³C frequencies of 75.5, 100.6 and 125.7 MHz respectively. Alternatively, ¹³C NMR measurements were performed on a BRUKER ACE 200 spectrometer at a frequency of 50.3 MHz.

Rotamers may or may not be denoted in spectra depending upon ease of interpretation of spectra. Unless otherwise stated, chemical shifts are given in ppm with the solvent as internal standard.

### Synthesis of Intermediates

### Example A

### 4-(2-Oxiranylmethoxy)benzonitrile

Epichlorohydrin (800 mL) and K₂CO₃ (414 g) were added to a stirred solution of p-cyanophenol (238 g) in 2.0 L MeCN and the reaction mixture was refluxed under an inert atmosphere for 2 h. The hot solution was filtered and the filtrate concentrated, giving a clear oil which was crystallized from di-iso-propyl ether giving the product in 75% yield.

¹³C NMR (CDCl₃): δ 44.4, 49.7, 69.0, 104.5, 115.3, 118.9, 134.0, 161.6

### Example B

### 2(S)-Oxiranylmethyl 3-nitrobenzenesulfonate

m-Nitrobenzensulfonylchloride (12.6 g; 57 mmol) was added to a cold (−20° C.) solution of (R)-(+)-glycidol (5.5 g; 74 mmol) and TEA (10.3 mL; 74 mmol). The reaction mixture was stirred at −20° C. for 96 h. The solution was filtered and the filtrate washed with tartaric acid (10% w/w), brine, H₂O and concentrated giving the title compound in a 97% yield.

¹H NMR (CDCl₃): δ 2.62 (dd, 1H), 2.84 (dd, 1H), 3.22 (m, 1H), 4.07 (dd, 1H), 4.49 (dd, 1H), 7.80 (t, 1H), 8.25 (m, 1H), 8.52 (m, 1H), 8.78 (m, 1H)

### Example C

### 4-[(2S)-Oxiranylmethoxy]benzonitrile

The title compound was prepared in a 90% yield according to the procedure described in Example A above starting from (R)-(−)-epichlorohydrin.

### Example D

### 4-[(2R)-Oxiranylmethoxy]benzonitrile

The title compound was prepared according to the procedure described in Example A above starting from (S)-(−)-epichlorohydrin.

[α]_{D}²⁰=−14.1° (c=1.0; acetone)

¹H NMR (CDCl₃): δ 2.79 (1H, m); 2.98 (1H, m); 3.39 (1H, m); 3.98 (1H, m); 4.37 (1H, m); 6.99 (2H, d); 7.60 (2H, d)

### Example E

### 3-Benzyl-3,7-diazabicyclo[3.3.1]nonane

(a) 3,7-Dibenzyl-3,7-diazabicyclo[3.3.1]nonane

The sub-title compound was prepared according to the method described in J. Org. Chem. 41, 1593, (1976) except that 3,7-dibenzyl-3,7-diazabicyclo[3.3.1]nonan-9-one (also prepared according to the method described in J. Org. Chem. 41, 1593 (1976)) was used instead of N-benzyl-N-methylbispidone.

(b) 3-Benzyl-3,7-diazazbicvclo[3.3.1]nonane

3,7-Dibenzyl-3,7-diazabicyclo[3.3.1]nonane (1.97 g; 6.4 mmol; from step (a) above) was dissolved in EtOH (95%) and hydrogenated over 5% Pd/C at 1 atm. until tlc indicated that the reaction was complete. The catalyst was removed by filtration through a pad of Celite® and the residue was concentrated under reduced pressure to give the title compound in a quantitative yield.

¹³C NMR (CDCl₃): δ 30.1, 33.4, 36.0, 52.5, 59.6, 64.3, 126.9, 128.3, 128.7, 138.8

### Example F

### tert-Butyl 3,7-diazabicyclo[3.3.1]nonane-3-carboxylate

(a) tert-Butyl 7-benzyl-9-oxy-3,7-diazabicyclo[3.3.1]nonane-3-carboxylate Paraformaldehyde (4.00 g; 127 mmol) was added to a solution of benzylamine (13.7 g; 126 mmol) in ethanol (190 mL). The solution was heated to 60° C. and a solution of acetic acid (15.2 g; 252 mmol) in ethanol (160 mL) was added over 2 hours. After additional stirring for 1 hour, the solution was cooled to room temperature. This solution was added (over 2 hours) to a mixture of 1-tert-butoxycarbonyl-4-piperidone (25.5 g; 127 mmol) and paraformaldehyde (4.80 g; 152 mmol) in ethanol (270 mL) which had been heated to 60° C. After reflux overnight, the solution was cooled to room temperature. The ethanol was removed by evaporation. Extractive work-up was performed in toluene:water and the material was filtered through silica in a toluene:ethyl acetate system. Evaporation of the eluant gave a solid material (37.4 g). The purity was 90 area % (HPLC) and the yield was 60%. By performing a crystallisation in iso-propanol, a compound with a purity of 98 area % (HPLC) and a yield of 70% was obtained.

MS (EI; 70 eV): m/z 91 (100%), m/z 57 (42%), m/z 273 (32%), m/z 330 (5%)

¹³C NMR (CDCl₃): δ 28.72, 47.71, 49.91, 50.60, 58.83, 59.16, 61.96, 80.18, 127.37, 128.45, 128.89. 137.57, 154.89, 213.66 (using TMS as reference)

(b) tert-Butyl 7-benzyl-9-oxy-3,7-diazabicyclo [3.3.1]nonane-3-carboxylate (alternative preparation)

Benzylamine (6.51 g; 60.2 mmol), acetic acid (72.3 g, 1200 mmol), paraformaldehyde (3.71 g; 120 mmol) and 1-tert-butoxycarbonyl-4-piperidone (12.0 g; 60.2 mmol), were added to ethanol (300 mL). The solution was heated to 65° C. and stirred at this temperature for 2 hours. The same work-up procedure as that described in step (a) above was performed, yielding 15.78 g of material with a purity of 92 area% (HPLC) and a yield of 70%. Recrystallisation from iso-propanol yielded a compound with a purity of 94 area % (HPLC) in a yield of 54%.

(c) tert-Butyl 7-benzyl-3,7-diazabicyclo[3.3.1]-nonane-3-carboxylate

A mixture of 4-toluenesulfonehydrazide (12.4 mmol; 2.30 g) and tert-butyl 7-benzyl-9-oxy-3,7-diazabicyclo[3.3.1]nonane-3-carboxylate (10.1 mmol; 4.00 g; 83.3%; from step (a) above) were dissolved in iso-propanol (30 mL) and heated at reflux for 2 hours. Acetic acid (2.5 mmol; 0.15 g) and sodium cyanoborohydride (12.1 mmol, 0.76 g) were added and the mixture was again heated at reflux for 2 hours. The slurry was cooled to ambient temperature and filtered. The filtrate was concentrated and an extractive work-up was performed in toluene:water. The toluene solution was concentrated to give 0.95 g of sub-title compound, with a purity of 90 area % (GC) in a yield of 60%.

MS (EI; 70 eV): m/z 259 (100%), m/z 91 (95%), m/z 169 (45%), m/z 57 (35%), m/z 316 (25%)

¹³C NMR (CDCl₃): δ 28.67, 28.95, 31.11, 47.55, 48.38, 58.70, 58.96, 63.46, 78.71, 126.57, 128.00, 128.53, 138.94, 155.20 (using TMS as a reference)

(d) tert-Butyl 3,7-diazabicyclo[3.3.1]nonane-3-carboxylate

tert-Butyl 7-benzyl-3,7-diazabicyclo[3.3.1]nonane-3-carboxylate (from step (c) above) was debenzylated according to the method described in Example E(b) above to give the title compound in quantitative yield.

¹³C NMR (CDCl₃): δ 28.05, 28.29, 31.33, 48.35, 49.11, 51.53, 79.34, 155.16

### Example G

### 4-[3-(3,7-Diazabicyclo[3.3.1]non-3-yl)-2-hydroxypropoxy]benzonitrile

HCl-saturated EtOAc (600 mL) was added to a solution of tert-butyl 7-[3-(4-cyanophenoxy)-2-hydroxypropyl]-3,7-diazabicyclo[3.3.1]nonane-3-carboxylate (62 g; see Example 2 of international patent application No. PCT/SE98/02276) in EtOAc (600 mL) and the mixture was stirred at rt. for 4 h. The solvent was removed under reduced pressure, the residue was dissolved in MeCN (1.3 L) and K₂CO₃ (100 g) was added. The suspension was stirred for 12 h and filtered. Concentration of the filtrate gave the title compound in a 90% yield.

¹³C NMR (CDCl₃): δ 28.9, 29.2, 32.3, 50.9, 57.7, 60.8, 62.1, 66.0, 71.2, 104.0, 115.3, 119.1, 133.9, 162.1

(The title compound was also readily converted to the hydrochloride salt using standard techniques.)

### Preparation of Compounds of Formula I

### Example 1

### 7-[(2S)-3-(4-Cyanophenoxy)-2-hydroxypropyl]-N-ethyl-3,7-diazabicyclo-[3.3.1]nonane-3-carboxamide

Ethyl isocyanate (1.42 g, 16.6 mmol) was added to a solution of 4-{[(2S)-3-(3,7-diazabicyclo[3.3.1]non-3-yl)-2-hydroxypropyl]oxy}benzonitrile) (5.0 g, 20 mmol, see Example G above) in 30 mL of dichloromethane. The mixture was stirred for 4 hours at room temperature and was then concentrated in vacuo and purified by column chromatography on silica, eluting with dichloromethane: methanol (95:5), to yield 3.2 g (51%) of the title compound.

¹³C NMR (CDCl₃): δ 15.52, 29.19, 29.50, 31.89, 35.77, 48.00, 49.17, 57.21, 60.49, 61.83, 65.41, 70.71, 103.88, 115.34, 119.15, 133.78, 133.84, 158.87, 162.19

### Example 2

### 7- [3-(4-Cyanophenoxy)-2-hydroxypropyl]-N-(cyclopropylmethyl)-3,7-diazabicyclo[3.3.1]nonane-3-carboxamide

(a) Cyclopropylmethyl isocyanate

Cyclopropylmethylamine (1.4 g, 19.7 mmol) was added to a suspension of 1,1′-carbonyldiimidazole (3.2 g, 19.7 mmol) in THF (10 mL). The resulting solution was stirred overnight at room temperature before being subjected to distillation, yielding 0.4 g (21%) of the sub-title compound.

(b) 7-[3-(4-Cyanophenoxy)-2-hydroxypropyl]-N-(cyclopropylmethyl)-3,7-diazabicyclo[3.3.1]nonane-3-carboxamide

Cyclopropylmethyl isocyanate (0.4 g, 4 mmol, from step (a) above) was added to a solution of 4-[3-(3,7-diazabicyclo[3.3.1]non-3-yl)-2-hydroxy-propoxy]benzonitrile (1.2 g, 4 mmol, see Example G above) in DCM. The solution was stirred overnight, then concentrated in vacuo. The resulting residue was purified by column chromatography on silica gel, eluting with dichloromethane:methanol (93:7), to yield 0.85 g (50%) of the title compound.

¹³C NMR (CDCl₃): δ 3.29, 11.21, 29.31, 29.61, 32.10, 46.11, 48.14, 49.39, 57.24, 60.58, 62.04, 65.46, 70.76, 104.03, 115.37, 119.18, 133.88, 158.97, 162.22

### Example 3

### 4-({(2S)-2-Hydroxy-3-[7-(4-morpholinylcarbonyl)-3,7-diazabicyclo-[3.3.1]non-3-yl]propyl}oxy)benzonitrile

A solution of 4-{[(2S)-3-(3,7-diazabicyclo[3.3.1]non-3-yl)-2-hydroxy-propyl]oxy}benzonitrile) (2.0 g, 6.6 mmol, prepared analogously to the method described in Example G above) in DCM (10 mL) was treated with aqueous NaOH (0.8 mL of 10 M), followed by 4-morpholinecarbonyl chloride (1.2 g, 8 mmol). The resulting mixture was stirred for 30 min. at room temperature, before water was added. The organic layer was separated, washed with 2 M NaOH followed by brine, before being separated, dried (MgSO₄) and concentrated in vacuo. The residue was recrystallised twice, firstly from iso-propanol and then from ethanol, to yield 0.73 g (26.5%) of the title compound.

¹³C NMR (CDCl₃): δ 23.36, 29.59, 30.05, 32.34, 47.45, 49.51, 52.18, 56.86, 60.78, 62.82, 65.35, 66.66, 70.82, 104.03, 115.33, 119.17, 133.88, 162.23, 164.99

### Example 4

### 7-{3-(4-Cyanophenoxy)-2-[(methanesulfonyl)amino]-propyl}-N-ethyl-3,7-diazabicyclo[3.3.1]nonane-3-carboxamide

(a) 4-(3-Amino-2-hydroxypropoxy)benzonitrile

4-(2-Oxiranylmethoxy)benzonitrile (100 g, 0.57 mol, see Example A above) was added to a mixture of concentrated aqueous ammonium hydroxide (500 mL) and iso-propanol (300 mL). The resulting slurry was stirred at room temperature for 3 days. The reaction mixture was filtered to remove the insoluble by-product, and the filtrate was concentrated in vacuo to give a crude product, which was crystallised from acetonitrile to yield 50 g (46%) of the sub-title compound.

(b) 2-(4-Cyanophenoxy)-1-{[(methanesulfonyl)amino]methyl}ethyl methanesulfonate

Methanesulfonyl chloride (17.5 g, 153 mmol) was slowly added to a cooled (−10° C.) solution of 4-(3-amino-2-hydroxypropoxy)benzonitrile (13.3 g, 69 mmol, from step (a) above) and 4-(dimethylamino)pyridine (0.2 g, 1.64 mmol) in pyridine (100 mL). The yellow solution was stirred at rt for 1.5 hours, concentrated in vacuo and then redissolved in DCM. This solution was washed twice with 2 M HCl and once with NaHCO₃ solution before the organic phase was separated, dried (MgSO₄) and concentrated in vacuo to yield 23.5 g (100%) of the sub-title compound.

(c) 4-{[1-(Methanesulfonyl)aziridin-2-yl]methoxy}benzonitrile

A stirred solution of 2-(4-cyanophenoxy)-1-{[(methanesulfonyl)amino]-methyl}ethyl methanesulfonate (23.5 g, 67 mmol, from step (b) above) in acetonitrile (200 mL), was treated with potassium carbonate (30 g, 210 mmol), forming a thick precipitate. After 1 hour, a further portion of K₂CO₃ (30 g, 210 mmol) was added. Stirring was continued for 2 h at rt before the reaction mixture was filtered and the filtrate concentrated in vacuo. The resulting oil (13 g) was crystallised from toluene to give 8 g (47%) of the sub-title compound.

mp 79-81° C.

(d) N-{2-(7-Benzyl-3,7-diazabicyclo[3.3.1]non-3-yl)- 1-[(4-cyanophenoxy)-methyl]ethyl}methanesulfonamide

A mixture of 3-benzyl-3,7-diazabicyclo[3.3.1]nonane (2 g, 10 mmol, see Example E above) and 4-{[1-(methanesulfonyl)aziridin-2-yl]methoxy}benzonitrile (2.5 g, 10 mmol, from step (c) above) in iso-propanol was refluxed overnight. The mixture was then concentrated in vacuo, giving a residue which was then dissolved in water (pH 3) and extracted with ether. The aqueous layer was made basic with 2 M NaOH and extracted with DCM. The dichloromethane layer was separated, dried and concentrated in vacuo to give a residue which was purified by column chromatography, eluting with a gradient of DCM:methanol:methanolic ammonia (98:2:0 to 97:0:3) to give 2.5 g (53%) of the sub-title compound.

(e) N-[2-(4-Cyanophenoxy)-1-(3,7-diazabicyclo[3.3.1]non-3-ylmethyl)-ethyl]methanesulfonamide

A solution of N-{2-(7-benzyl-3,7-diazabicyclo[3.3.1]non-3-yl)-1-[(4-cyanophenoxy)methyl]ethyl}methanesulfonamide (2.3 g 4.9 mmol, from step (d) above) in aqueous ethanol (95%; 55 mL) was hydrogenated over 5% Pd/C at ambient pressure. The catalyst was removed by filtration through a pad of Celite® and the residue was concentrated in vacuo to give 1.6 g of a crude product. This was recrystallised from methanol to yield 0.3 g (16%) of the sub-title compound.

(f) 7-{3-(4-Cyanophenoxy)-2-[(methanesulfonyl)amino]propyl}-N-ethyl-3,7-diazabicyclo[3.3.1]nonane-3-carboxamide

A suspension of N-[2-(4-cyanophenoxy)-1-(3,7-diazabicyclo[3.3.1]non-3-ylmethyl)ethyl]methanesulfonamide (0.29 g, 0.77 mmol, from step (e) above) in DCM (10 mL) was treated with ethyl isocyanate (66 μL, 0.84 mmol) to give a clear solution. The mixture was stirred for 1 h at rt, concentrated in vacuo and then purified by column chromatography, eluting with 5% MeOH in DCM, to give the title compound in 73% yield.

¹³C NMR (CDCl₃): δ 15.41, 28.88, 29.18, 30.77, 35.87, 41.78, 47.93, 48.65, 49.98, 58.24, 58.51, 60.15, 68.82, 104.51, 115.28, 118.95, 134.05, 158.58, 161.55

### Example 5

### 7-[(2S)-3-(4-Cyanophenoxy)-2-hydroxypropyl]-N-iso-propyl-3,7-diazabicyclo[3.3.1]nonane-3-carboxamide

(a) 7-Benzyl-N-iso-propyl-3,7-diazabicyclo[3.3.1]nonane-3-carboxamide

iso-Propyl isocyanate (1.7 g, 20 mmol) was slowly added to a solution of 3-benzyl-3,7-diazabicyclo[3.3.1]nonane (3.1 g, 14.3 mmol, see Example E above) in DCM (10 mL). The mixture was stirred at rt overnight and then concentrated in vacuo to yield 4.2 g (97%) of the sub-title compound.

(b) N-iso-Propyl-3,7-diazabicyclo [3.3.1]nonane-3-carboxamide

A solution of 7-benzyl-N-iso-propyl-3,7-diazabicyclo[3.3.1]nonane-3-carboxamide (4.2 g, 14 mmol, from step (a) above) in methanol/water (17 mL of a 15:2 mixture) was hydrogenated over 5% Pd/C at ambient pressure. The catalyst was removed by filtration through a pad of Celite®, and the filtrate concentrated in vacuo to yield 2.6 g (87%) of the sub-title compound.

(c) 7-[(2S)-3-(4-Cyanophenoxy)-2-hydroxypropyl]-N-iso-propyl-3,7-diaza-bicyclo[3.3.1]nonane-3-carboxamide

A mixture of 4-[(2S)-oxiranylmethoxy]benzonitrile (0.55 g, 3.14 mmol, see Example C above) and N-iso-propyl-3,7-diazabicyclo[3.3.1]nonane-3-carboxamide (0.85 g, 4 mmol, from step (b) above) in iso-propanol/water (6.5 mL of a 12:1 mixture) was stirred overnight at 60° C. The mixture was then concentrated in vacuo and the residue re-dissolved in DCM. The organic solution was washed with water then brine, dried (MgSO₄) and concentrated in vacuo to give the title compound in 91% yield.

¹³C NMR (CDCl₃): δ 23.49, 29.29, 31.78, 42.26, 47.71, 49.09, 56.92, 60.27, 61.65, 65.19, 70.61, 103.54, 115.21, 119.09, 133.65, 158.11, 162.08

### Example 6

### 7-[(2R)-3-(4-Cyano-2-{[(2-cyanoethyl)amino]carbonyl}-phenoxy)-2-hydroxypropyl]-N-ethyl-3,7-diazabicyclo[3.3.1]nonane-3-carboxamide

(a) 7-Benzyl-N-ethyl-3,7-diazabicyclo[3.3.1]nonane-3-carboxamide

A cooled (0° C.) solution of 3-benzyl-3,7-diazabicyclo[3.3.1]nonane (32.45 g, 0.15 mol, see Example E above) in DCM (300 mL) was treated with ethyl isocyanate (11.4 g, 0.16 mol), added dropwise. The solution was stirred for 2 h at rt before being concentrated in vacuo. The resulting residue was purified by chromatography on silica gel, eluting with a gradient of DCM:MeOH (100:0 to 90:10) to yield 36.4 g (84%) of the sub-title compound.

(b) N-Ethyl-3,7-diazabicyclo[3.3.1]nonane-3-carboxamide

A solution of 7-benzyl-N-ethyl-3,7-diazabicyclo[3.3.1]nonane-3-carboxamide (4.4 g, 15.3 mmol, from step (a) above) in aqueous ethanol (25 mL of 95%) was hydrogenated over 5% Pd/C at ambient pressure. The catalyst was removed by filtration through a pad of Celite®, and the residue was concentrated in vacuo to yield 2.88 g (95%) of the sub-title compound.

(c) Methyl 5-bromo-2-hydroxybenzoate

Br₂ (52 g) was slowly added to a stirred solution of methyl salicylate (50 g; 330 mmol) in 300 mL acetic acid. The reaction mixture was stirred at rt. for 10 h, poured onto ice-water and the precipitate recrystallized from MeOH, giving the sub-title compound in a 83% yield.

(d) Methyl 5-cyano-2-hydroxybenzoate

Methyl 5-bromo-2-hydroxybenzoate (190.8 g; from step (c) above) and CuCN (73.9 g) were refluxed in DMF (500 mL) for 7 h. The temperature was allowed to decrease to 80° C. and HCl (500 mL) and FeCl₃ (165.0 g) were added. The reaction mixture was stirred for 30 min., concentrated and partitioned between H₂O and DCM. The organic layer was dried, concentrated the residue recrystallized from methylethyl ketone giving the sub-title compound in a 61% yield.

(e) 5-Cyano-N-(2-cyanoethyl)-2-hydroxybenzamide

A mixture of methyl 5-cyano-2-hydroxybenzoate (20 g, 0.113 mol, from step (d) above), 3-aminopropanenitrile (15.4 g, 0.22 mol) and sodium cyanide (1 g, 20 mmol) in methanol (200 mL) was refluxed overnight. Tlc showed incomplete reaction, so DMSO (50 mL) was added, and reflux was continued for a further 5 h. The solution was concentrated in vacuo, water added, followed by conc. HCl, until a precipitate formed. The product was filtered off, washed with water and dried to yield 19.4 g (80%) of the sub-title compound.

(f) 5-Cyano-N-(2-cyanoethyl)-2-[(2R)-oxiranylmethoxylbenzamide

A mixture of 5-cyano-N-(2-cyanoethyl)-2-hydroxybenzamide (2.1 g, 9.8 mmol, from step (e) above) and 10 equivalents of (S)-epichlorohydrin in iso-propanol:water (55 mL of 10:1) was refluxed overnight. The mixture was concentrated in vacuo and the residue purified by column chromatography, eluting with ethyl acetate to yield 0.63 g (24%) of the sub-title compound.

(g) 7-[(2R)-3-(4-Cyano-2-{[(2-cyanoethyl)amino]carbonyl}phenoxy)-2-hydroxypropyl]-N-ethyl-3,7-diazabicyclo[3.3.1]nonane-3-carboxamide

A mixture of 5-cyano-N-(2-cyanoethyl)-2-[(2R)-oxiranylmethoxy]-benzamide (0.63 g, 2.3 mmol, from step (f) above) and N-ethyl-3,7-diazabicyclo[3.3.1]nonane-3-carboxamide (0.59 g, 3 mmol, from step (b) above) in iso-propanol:water (33 mL of 10:1) was stirred under reflux overnight. The reaction mixture was concentrated in vacuo and the residue purified by column chromatography, eluting with DCM:MeOH (9:1), to yield 0.78 g (73%) of the title compound.

¹³C NMR (CDCl₃): δ 15.40, 15.55, 17.94, 28.04, 29.21, 29.55, 31.31, 32.03, 35.69, 35.89, 36.21, 47.93, 48.65, 49.36, 57.00, 60.47, 61.05, 65.32, 72.21, 105.39, 114.37, 118.22, 118.45, 123.28, 136.36, 136.45, 158.53, 159.20, 160.08, 163.75

ES-MS (M+1)⁺469.0 (m/z)

### Example 7

### 7-((2S)-3-{4-Cyano-2-[(cyclopropylamino)carbonyl]-phenoxy}-2-hydroxypropyl)-N-ethyl-3,7-diazabicyclo[3.3.1]nonane-3-carboxamide

(a) N¹-Cyclopropyl-5-cyano-2-hydroxybenzamide

Cyclopropylamine (14.3 g) and Na (100 mg) were added to a solution of methyl 5-cyano-2-hydroxybenzoate (10.0 g; from step (d) above) in DMSO (40 mL). The reaction mixture was heated at 80° C. in a sealed steel vessel overnight, diluted with H₂O, acidified and extracted with EtOAc, giving the sub-title compound (11.0 g), after concentration of the organic layer.

(b) 5-Cyano-N-cyclopropyl-2-[(2S)-oxiranylmethoxy]benzamide

A mixture of N¹-cyclopropyl-5-cyano-2-hydroxybenzamide (1.56 g, 7.7 mmol, from step (a) above), (2S)-oxiranylmethyl 3-nitrobenzene-sulfonate (2 g, 7.7 mmol, see Example B above) and K₂CO₃ (1.16 g, 8.4 mmol) in 2-butanone (15 mL) was stirred at 60° C. for 18 h. The mixture was concentrated in vacuo and the residue crystallised from di-iso-propyl ether:MeCN (9:1) to yield 0.97 g (97%) of the sub-title compound.

(c) 7-((2S)-3-{4-Cyano-2-[(cyclopropylamino)carbonyl]phenoxyl}-2-hydroxypropyl)-N-ethyl-3,7-diazabicyclo [3.3.1]nonane-3-carboxamide

A mixture of 5-cyano-N-cyclopropyl-2-[(2S)-oxiranylmethoxy]benzamide (0.97 g, 3.8 mmol, from step (b) above) and N-ethyl-3,7-diazabicyclo-[3.3.1]nonane-3-carboxamide (0.89 g, 4.5 mmol, see Example 6(b) above) in iso-propanol:water (22 mL of 10:1) was refluxed overnight. The solvent was removed in vacuo and the resulting residue purified by column chromatography on silica gel, eluting with DCM:MeOH (9:1), to yield 1.37 g (79%) of the title compound.

¹³C NMR (CDCl₃): δ 6.62, 6.78, 15.81, 23.55, 29.61, 29.90, 32.48, 36.20, 48.32, 49.84, 53.68, 57.48, 60.92, 62.06, 65.61, 71.72, 105.42, 113.69, 118.64, 123.78, 136.26, 136.77, 159.70, 159.97, 164.75

### Example 8

### N-Ethyl-7-(4-nitrophenethyl)-3,7-diazabicyclo[3.3.1]nonane-3-carboxamide

A mixture of 1-(2-bromoethyl)-4-nitrobenzene (1.6 g, 7.0 mmol), N-ethyl-3,7-diazabicyclo[3.3.1]nonane-3-carboxamide (1.0 g, 5.1 mmol, see Example 6(b) above) and K₂CO₃ (1.38 g, 10 mmol) was stirred at rt overnight. The mixture was then filtered and concentrated in vacuo and the resulting residue purified by column chromatography, eluting with a gradient of DCM:MeOH (100:0 to 90:10), to yield 1.5 g (85%) of the title compound.

¹³C NMR (CDCl₃): δ 15.71, 28.83, 30.11, 33.03, 35.67, 47.97, 59.22, 20 59.49, 123.34, 129.65, 146.26, 149.15, 157.95

### Example 9

### N-(Cyanomethyl)-7-[(2S)-3-(4-cyanophenoxy)-2-hydroxypropyl]-3,7-diaza-bicyclo[3.3.1]nonane-3-carboxamide

(a) Cyanomethyl Isocyanate

The title compound was prepared according to the procedure described in Example 2(a) above, using 2-aminoacetonitrile in place of cyclopropylmethylamine.

(b) N-(Cyanomethyl)-7-[(2S)-3-(4-cyanophenoxy)-2-hydroxypropyl]-3,7-diazabicyclo[3.3.1]nonane-3-carboxamide

The title compound was prepared in 26% yield (counting steps (a) and (b) together) according to procedure described in Example 2(b) above, using cyanomethyl isocyanate (from step (a) above) in place of cyclopropylmethyl isocyanate.

¹³C NMR (CDCl₃): δ 28.99, 29.27, 29.47, 31.77, 48.32, 49.33, 56.88, 60.33, 61.61, 65.32, 70.63, 103.96, 115.31, 117.63, 119.21, 133.93, 157.74, 162.08

### Example 10

N-Ethyl-7-{4-[(methanesulfonyl)amino]phenethyl}-3,7-diazabicyclo-[3.3.1]nonane-3-carboxamide

(a) 4-[(Methanesulfonyl)amino]phenethyl methanesulfonate

Methanesulfonyl chloride (45 g, 0.39 mol) was added, dropwise over 30 minutes, to a cooled (−5° C.) solution of 4-aminophenethyl alcohol (25.2 g, 0.18 mol) in pyridine (200 mL). The mixture was stirred at 0° C. for 1 h and then at rt overnight. The resulting red suspension was poured in to a mixture of ice (300 mL) and conc. HCl (60 mL). The pink precipitate that formed was filtered off, redissolved in DCM, dried and treated with activated carbon. The resulting solution was concentrated in vacuo to give a residue, which, on recrystallisation from ethyl acetate, gave 34.5 g (64%) of the sub-title compound.

mp 133-134° C.

(b) N-Ethyl-7-{4-[(methanesulfonyl)amino]phenethyl}-3,7-diazabicyclo-[3.3.1]nonane-3-carboxamide

A mixture of N-ethyl-3,7-diazabicyclo[3.3.1]nonane-3-carboxamide (1 g, mmol, see Example 6(b) above), 4-[(methanesulfonyl)amino]phenethyl methanesulfonate (1.5 g, 5 mmol, from step (a) above) and NaHCO₃ (3 g, 35.7 mmol) in MeCN (50 mL) was refluxed for 3 h under nitrogen. The reaction mixture was filtered and concentrated in vacuo to give 2.2 g of crude product, which was filtered through a silica plug, with MeOH/2 N HCl. The pH of the fractions was raised to pH 6 and extracted with DCM, yielding 0.2 g of the tide compound.

¹³C NMR (CDCl₃): δ 15.75, 28.87, 30.23, 32.58, 35.64, 35.76, 39.14, 48.18, 59.17, 60.26, 121.41, 129.85, 134.72

### Example 11

### 7-[3-(4-Cyanophenoxy)-2-fluoropropyl]-N-ethyl-3,7-diazabicyclo-[3.3.1]nonane-3-carboxamide

(a) 7-[3-(4-Cyanophenoxy)-2-hydroxypropyl]-N-ethyl-3,7-diazabicyclo-[3.3.1]nonane-3-carboxamide

The title compound was prepared according to the procedure described in Example 1 above, using 4-[3-(3,7-diazabicyclo[3.3.1]non-3-yl)-2-hydroxypropoxy]benzonitrile (see Example G above) in place of 4-{[(2S)-3-(3,7-diazabicyclo[3.3.1]non-3-yl)-2-hydroxypropyl]oxy}benzonitrile.

(b) 7-[3-(4-Cyanophenoxy)-2-fluoropropyl]-N-ethyl-3,7-diazabicyclo-3.3.1]nonane-3-carboxamide

A solution of 7-[3-(4-cyanophenoxy)-2-hydroxypropyl]-N-ethyl-3,7-diaza-bicyclo[3.3.1]nonane-3-carboxamide (1.0 g, 2.7 mmol, from step (a) above) in DCM (2.5 mL) was cooled to −78° C. A solution of (diethylamino)sulfurtrifluoride in DCM (2.5 mL) was added slowly under stirring. Stirring was continued for 35 minutes, during which time the reaction was allowed to warm to room temperature. Dicloforomethane was added and the reaction mixture was then washed with NaHCO₃, dried and concentrated in vacuo. The resulting residue was purified by column chromatography, eluting with DCM:MeOH (98:2), to yield 0.68 g (67%) of the title compound.

¹³C NMR (CDCl₃): δ 15.63, 29.00, 30.33, 35.70, 47.78, 47.93, 58.36, 58.67, 59.82, 60.39, 68.60, 68.89, 89.56, 91.86, 104.15, 115.56, 119.25, 133.97, 157.61, 161.92

### Example 12

### 7-[3-(4-Cyanophenoxy)-2-hydroxypropyl]-N-[2-oxo-2-(propylamino)-ethyl]-3,7-diazabicyclo[3.3.1]nonane-3-carboxamide

(a) Ethyl 2-[({7-[3-(4-cyanophenoxy)-2-hydroxypropyl]-3,7-diazabicyclo-[3.3.1]non-3-yl}carbonyl)amino]acetate

A cooled (0° C.) solution of 4-[3-(3,7-diazabicyclo[3.3.1]non-3-yl)-2-hydroxypropoxy]benzonitrile (23.1 g, 77 mmol, see Example G above) in DCM (700 mL) was treated with ethyl 2-isocyanatoacetate (9.92 g, 77 mmol), and then stirred at rt for 7 h. The reaction mixture was concentrated in vacuo to yield 33.6 g (100%) of the sub-title compound.

(b) 7-[3-(4-Cyanophenoxy)-2-hydroxypropyl]-N-[2-oxo-2-(propylamino)-ethyl]-3,7-diazabicyclo[3.3.1]nonane-3-carboxamide

A mixture of ethyl 2-[({7-[3-(4-cyanophenoxy)-2-hydroxypropyl]-3,7-diazabicyclo[3.3.1]non-3-yl}carbonyl)amino]acetate (0.76 g 1.8 mmol, from step (a) above), propylamine (5 mL, 3.6 g, 69.1 mmol) and NaCN (0.01 g, 0.2 mmol) in methanol (10 mL) was warmed to 75° C. in a sealed tube overnight. The solvent was then removed in vacuo and the residue diluted with Na₂CO₃ solution. The aqueous mixture was extracted with DCM, and the resulting organic layer separated, dried and concentrated in vacuo. The resulting residue was purified by column chromatography, eluting with a gradient of dichloromethane:methanol (100:0 to 90:10), to give the title compound in 70% yield.

¹³C NMR (CDCl₃): δ 11.36, 22.65, 29.12, 29.42, 31.78, 41.15, 44.75, 48.15, 49.10, 56.99, 60.40, 61.35, 65.33, 70.74, 103.99, 115.27, 119.12, 133.91, 158.71, 162.10, 170.62

### Example 13

### 7-{3-(4-Cyanophenoxy)-2-[(4-morpholinylcarbonyl)amino]propyl}-N-ethyl-3,7-diazabicyclo[3.3.1]nonane-3-carboxamide

(a) tert-Butyl 3-(4-cyanophenoxy)-2-hydroxypropylcarbamate

A cooled (0° C.) solution of 4-(3-amino-2-hydroxypropoxy)benzonitrile (44.6 g, 0.23 mol, see Example 4(a) above) in THF:H₂0. (1.5 L of 1:1) was treated with di-tert-butyl dicarbonate (53 g, 0.24 mol). The mixture was stirred at rt overnight, after which NaCl was added and the resulting organic layer separated. The water layer was extracted with ether and the combined organics were dried and concentrated in vacuo. The resulting oil (70 g) was filtered through a plug of silica, and then crystallised from diethyl ether:di-iso-propyl ether to yield 50 g of the sub-title compound.

(b) 2-[(tert-Butoxycarbonyl)amino]-1-[(4-cyanophenoxy)methyl]ethyl methanesulfonate

Methanesulfonyl chloride (22.3 g 0.195 mol) was added over the course of 1.5 hours to a cooled (0° C.) solution of tert-butyl 3-(4-cyanophenoxy)-2-hydroxypropylcarbamate (51.2 g, 0.177 mol, from step (a) above) and 4-(dimethylamino)pyridine (1.3 g, 10.6 mmol) in pyridine (250 mL), kept under an inert atmosphere. The reaction mixture was stirred for 2 h at rt before water and DCM were added. The organic layer was separated, washed with water, dried (MgSO₄) and concentrated in vacuo to yield 68.1 g (100%) of the sub-title compound.

(c) tert-Butyl 2-[(4-cyanophenoxy)methyl]-1-aziridinecarboxylate

A cooled (0° C.) solution of 2-[(tert-butoxycarbonyl)amino]-1-[(4-cyano-phenoxy)methyl]ethyl methanesulfonate (30.6 g, 82.6 mmol, from step (b) above) and tetrabutylammonium hydrogensulfate (3 g, 8.8 mmol) in DCM (100 mL) was treated with 50 wt. % aqueous NaOH (60 mL) under an inert atmosphere. The resulting mixture was stirred, and the temperature was slowly allowed to rise to rt over for 4 h, and then extracted with ether. The organic layer was washed with water and concentrated in vacuo to give a residue that was purified by column chromatography (dichloromethane eluant). Crystallisation from diethyl ether:di-iso-propyl ether gave the sub-title compound in quantitative yield.

(d) tert-Butyl 2-(4-cyanophenoxy)-1-({7-[(ethylamino)carbonyl]-3,7-diaza-bicyclo[3.3.1]non-3-yl}methyl)ethylcarbamate

A mixture of N-ethyl-3,7-diazabicyclo[3.3.1]nonane-3-carboxamide (2.88 g, 14.6 mmol, see Example 6(b) above) and tert-butyl 2-[(4-cyanophenoxy)methyl]-1-aziridinecarboxylate (4.0 g, 14.6 mmol, from step (c) above) in iso-propanol (20 mL) was refluxed overnight. The reaction mixture was concentrated in vacuo to give 7.4 g of a yellow oil, which was purified by column chromatography, eluting with a gradient of DCM:MeOH (100:0 to 90:10), to yield 3.33 g of the sub-title compound.

(e) 7-[2-Amino-3-(4-cyanophenoxy)propyl]-N-ethyl-3,7-diazabicyclo-[3.3.1]nonane-3-carboxamide

A solution of tert-butyl 2-(4-cyanophenoxy)-1-({7-[(ethylamino)carbonyl]-3,7-diazabicyclo[3.3.1]non-3-yl}methyl)ethylcarbamate (2.4 g, 5.1 mmol, from step (d) above) in HCl-saturated ethyl acetate was stirred for 1 h at rt. The reaction mixture was then concentrated in vacuo and resulting residue re-dissolved in water. The aqueous solution was treated with aqueous NaHCO₃ and extracted with DCM, which organic layer was then dried and concentrated in vacuo to give 2 g of the sub-title compound.

(f) 7-{3-(4-Cyanophenoxy)-2-[(4-morpholinylcarbonyl)amino]propyl}-N-ethyl-3,7-diazabicyclo[3.3.1]nonane-3-carboxamide

A cooled (5° C.) solution of 7-[2-amino-3-(4-cyanophenoxy)propyl]-N-ethyl-3,7-diazabicyclo[3.3.1]nonane-3-carboxamide (0.33 g, 0.7 mmol, from step (e) above) and triethylamine (0.4 mL, 3.0 mmol) in DCM (5 mL) was treated with 4-morpholinecarbonyl chloride (0.11 g, 0.7 mmol), and then stirred at 5° C. for 3 h. After further stirring at room temperature overnight, tlc analysis indicated incomplete reaction, and so a further portion of 4-morpholinecarbonyl chloride (40 mg, 0.27 mmol) was added. Stirring was continued at rt overnight again before NaHCO₃ solution was added. The organic layer was separated, dried and concentrated in vacuo to give 400 mg of crude product, which was purified by column chromatography on silica gel, eluting with dichloromethane:methanolic ammonia (95:5) to give 250 mg of the title compound.

¹³C NMR (CDCl₃): δ 161.94, 158.26, 157.81, 133.94, 119.15, 115.37, 103.90, 67.26, 66.66, 60.66, 60.51, 57.99, 48.93, 48.37, 47.39, 44.06, 35.93, 30.71, 29.34, 29.02, 15.51

### Example 14

### N-(4-Cyanophenethyl)-7-(4-oxoheptyl)-3,7-diazabicyclo[3.3.1]nonane-3-carboxamide

(a) 3-Benzyl-7-[3-(2-propyl-1,3-dioxolan-2-yl)propyl]-3,7-diazabicyclo-[3.3.1]nonane

A mixture of 3-benzyl-3,7-diazabicyclo[3.3. 1]nonane (10.5 g, 48.5 mmol, see Example E above), 2-(3-bromopropyl)-2-propyl-1,3-dioxolane (11.5 g, 48.5 mmol, Bajrowicsz et al., Tetrahedron, 41 (1985) 1833) and K₂CO₃ (13.8 g, 0.1 mol) in MeCN (50 mL) was refluxed overnight. The reaction mixture was filtered and concentrated in vacuo to yield 18.8 g (100%) of the sub-title compound.

(b) 3-[3-(2-Propyl-1,3-dioxolan-2-yl)propyl]-3,7-diazabicyclo[3.3.1]-nonane

A solution of 3-benzyl-7-[3-(2-propyl-1,3-dioxolan-2-yl)propyl]-3,7-diazabicyclo[3.3.1]nonane (18.8 g, 4.85 mmol, from step (a) above) in ethanol (100 mL) was hydrogenated over 5% Pd/C at ambient pressure. The catalyst was removed by filtration through a pad of Celite®, and the filtrate concentrated in vacuo to yield 13.7 g (100%) of the sub-title compound.

(c) N-(4-Cyanophenethyl)-7-(4-oxoheptyl)-3,7-diazabicyclo[3.3.1]nonane-3-carboxamide

A solution of 4-(2-aminoethyl)benzonitrile (1.0 g, 6.9 mmol, Wiley et al., Bioorg. Med. Chem. Lett., 6 (1996) 2387) in dry THF (10 mL) was treated with 1,1′-carbonyldiimidazole (1.17 g, 7.2 mmol), and the mixture was stirred for 30 min. A solution of 3-[3-(2-propyl-1,3-dioxolan-2-yl)propyl]-3,7-diazabicyclo[3.3.1]nonane (1.3 g, 4.6 mmol, from step (b) above) in THF (5 mL) was added to the reaction mixture, and stirring was continued overnight at rt. The solution was then concentrated in vacuo and the resulting residue diluted with MeOH and 2 M HCl, which solution was stirred for 2 h at rt. The mixture was made alkaline and extracted with DCM. The organic layer was separated, dried and concentrated in vacuo to give a residue which was purified by flash chromatography, eluting with DCM:MeOH (92:8), to yield 0.57 g (30%) of the title compound.

¹³C NMR (CDCl₃): δ 13.73, 17.21, 20.85, 28.79, 30.38, 36.91, 39.84, 41.83, 44.73, 47.94, 57.65, 59.05, 110.06, 118.93, 129.67, 132.20, 145.52, 157.47, 211.67

### Example 15

### N′-(4-Cyanobenzoyl)-7-(4-oxoheptyl)-3,7-diazabicyclo[3.3.1]nonane-3-carbohydrazide

A mixture of 4-cyanobenzohydrazide (0.82 g, 5.0 mmol) and 1,1′-carbonyldiimidazole (0.82 g, 5 mmol) in THF (15 mL) was stirred for 10 min at rt before 3-[3-(2-propyl-1,3-dioxolan-2-yl)propyl]-3,7-diazabicyclo-[3.3.1]nonane (1.44 g, 5.0 mmol, see Example 14(b) above) was added. The reaction mixture was stirred overnight at rt, before being concentrated in vacuo. The resulting residue was dissolved in DCM, and washed with water. The organic layer was separated and concentrated in vacuo to give a residue which was dissolved in methanol/2M HCl. Evaporation of the MeOH in vacuo and extraction of the remaining aqueous solution with DCM, gave, after purification by flash chromatography on silica gel (dichloromethane:methanolic ammonia eluant), 0.5 g (25%) of the title compound.

¹³C NMR (CDCl₃): δ 213.21, 164.24, 157.01, 136.31, 132.19, 128.24, 118.11, 115.11, 58.65, 57.89, 48.38, 44.31, 40.55, 31.52, 29.12, 21.60, 17.08, 13.69

### Example 16

### 4-{2-Amino-3-[7-(1-piperidinylcarbonyl)-3,7-diazabicyclo[3.3.1]non-3-yl]propoxy}benzonitrile

(a) 7-Benzyl-3,7-diazabicyclo[3.3.1]non-3-yl(1-piperidinyl)methanone

The sub-title compound was prepared by way of a reaction between 3-benzyl-3,7-diazabicyclo[3.3.1]nonane (see Example E above) and 1-piperidinecarbonyl chloride (Boon, J. Chem. Soc., (1947) 307, 313).

(b) 3,7-Diazabicyclo[3.3.1]non-3-yl(1-piperidinyl)methanone

The sub-title compound was obtained in quantitative yield according to the procedure described in Example 14(b) above, using 7-benzyl-3,7-diazabicyclo[3.3.1]non-3-yl(1-piperidinyl)methanone (from step (a) above) in place of 3-benzyl-7-[3-(2-propyl-1,3-dioxolan-2-yl)propyl]-3,7-diazabicyclo[3.3.1]nonane.

(c) tert-Butyl 2-(4-cyanophenoxy)-1-{[7-(1-piperidinylcarbonyl)-3,7-diaza-bicyclo[3.3.1]non-3-yl]methyl}ethylcarbamate

A mixture of tert-butyl 2-[(4-cyanophenoxy)methyl]-1-aziridinecarboxylate (1.92 g, 7 mmol, see Example 13(c) above) and 3,7-diaza-bicyclo[3.3.1]non-3-yl(1-piperidinyl)methanone (1.85 g, 7 mmol, from step (a) above) in iso-propanol (15 mL) was refluxed for 30 h. The solution was concentrated in vacuo to yield 3.7 g of crude product, which was purified by chromatography using. 2.5% MeOH in DCM to give 2.0 g (56%) of sub-title compound.

(d) 4-{2-Amino-3-[7-(1-piperidinylcarbonyl)-3,7-diazabicyclo[3.3.1]non-3-yl]propoxy}benzonitrile

A cooled (0° C.) solution of tert-butyl 2-(4-cyanophenoxy)-1-{[7-(1-piperidinylcarbonyl)-3,7-diazabicyclo[3.3.1]non-3-yl]methyl}ethyl-carbamate (1.9 g, 3.7 mmol, from step (c) above) in ethyl acetate was treated with HCl-saturated ethyl acetate. The mixture was stirred for 4 h before being concentrated in vacuo. The resulting residue was dissolved in water, made basic with NaHCO₃ and extracted with DCM. The organic layer was separated, dried and concentrated in vacuo to yield 1.5 g (100%) of the title compound.

¹³C NMR (CDCl₃): δ 24.73, 25.72, 29.62, 29.95, 32.11, 47.44, 48.14, 49.53, 50.98, 57.87, 60.57, 62.59, 72.03, 103.90, 115.30, 119.22, 133.91, 162.23, 164.35

### Example 17

### N-Ethyl-7-{2-hydroxy-3-[4-(1H-imidazol-1-yl)phenoxy]propyl}-3,7-diazabicyclo[3.3.1]nonane-3-carboxamide

(a) 1-[4-(2-Oxiranylmethoxy)phenyl]-1H-imidazole

A mixture of 4-(1H-imidazol-1-yl)phenol (10 g, 60 mmol), K₂CO₃ (8.63 g, 60 mmol) and 2-oxiranylmethyl 3-nitrobenzenesulfonate (15.5 g, 60 mmol, see Example B above) in DMF (140 mL) was stirred at 40° C. overnight. The mixture was then concentrated in vacuo and the resulting residue diluted with DCM, washed with water, dried and then concentrated in vacuo. The crude product was then purified by flash chromatography, eluting with a gradient of dichloromethane:methanol (100:0 to 70:30) to yield 3.4 g, (72.6%) of the title compound.

(b) N-Ethyl-7-{2-hydroxy-3-[4-(1H-imidazol-1-yl)phenoxy]propyl}-3,7-diazabicyclo[3.3.1]nonane-3-carboxamide

A mixture of 1-[4-(2-oxiranylmethoxy)phenyl]-b 1H-imidazole (3.16 g, 14.6 mmol, from step (a) above) and N-ethyl-3,7-diazabicyclo-[3.3.1]nonane-3-carboxamide (2.88 g 14.6 mmol, see Example 6(b) above) in iso-propanol:H₂O (18 mL of 9:1) was refluxed for 3 hours, concentrated in vacuo and purified by acid/base extraction to yield 4.4 g (72.6%) of the title compound.

¹³C NMR (CDCl₃): δ 15.52, 29.13, 29.44, 31.84, 35.70, 47.92, 49.07, 57.21, 60.44, 61.94, 65.45. 70.76, 115.49, 118.58, 122.90, 129.86, 130.56, 135.66, 158.16, 158.78

### Example 18

### N-[3-(4-Cyanophenoxy)propyl]-7-(2-hydroxyethyl)-3,7-diazabicyclo-[3.3.1]nonane-3-carboxamide

(a) 4-(3-Bromopropoxy)benzonitrile

1,3-Dibromopropane (1.02 L; 10 mol) was added to a stirred suspension of p-cyanophenol (238 g; 2 mol), K₂CO₃ (276.4 g; 2 mol) in MeCN (2.7 L). The reaction mixture was refluxed for 4 h, filtered and concentrated. The residue was recrystallized from iso-propyl ether to give the sub-title compound in a 69% yield.

(b) 4-[3-(1,3-Dioxo-1,3-dihydro-2H-isoindol-2-yl)propoxy]benzonitrile

A mixture of 4-(3-bromopropoxy)benzonitrile (20 g, 84 mmol, see step (a) above) and potassium phthalimide (15.5 g, 84 mmol) in DMF (120 mL) was stirred at 95° C. for 4 h. The solution was then concentrated in vacuo and the resulting residue dissolved in DCM and washed with water. The organic layer was separated, dried (Na₂SO₄) and concentrated in vacuo to yield 25.5 g (99%) of the sub-title compound.

(c) 4-(3-Aminopropoxy)benzonitrile

A mixture of 4-[3-(1,3-dioxo-1,3-dihydro-2H-isoindol-2-yl)propoxy]-benzonitrile (25.5 g, 83 mmol, from step (b) above) and hydrazine hydrate (4.15 g, 83 mmol) in methanol (100 mL) was refluxed for 1 h before water (120 mL) was added. The methanol was evaporated under reduced pressure and concentrated hydrochloric acid (120 mL) was added. The resulting mixture was heated on a steam bath for 1.5 h and then cooled in the refrigerator overnight. The resulting precipitate was filtered off and the filtrate was concentrated in vacuo. Water was added to the resulting residue and the solution made basic. The aqueous solution was extracted with DCM, which organic layer was then separated, dried and concentrated in vacuo to yield 6 g (41%) of the sub-title compound.

(d) 7-Benzyl-3,7-diazabicyclo[3.3.1]nonane-3-ethanol

The compound was prepared in 72% yield by reacting 3-benzyl-3,7-diazabicyclo[3.3.1]nonane (see Example E above) with 2-bromoethanol.

(e) 3,7-Diazabicyclo[3.3.1]nonane-3-ethanol

The sub-title compound was prepared according to the procedure described in Example 14(b) above, using 7-benzyl-3,7-diazabicyclo[3.3.1]nonane-3-ethanol (from step (d) above) in place of 3-benzyl-7-[3-(2-propyl-1,3-dioxolan-2-yl)propyl]-3,7-diazabicyclo[3.3.1]-nonane.

(f) N-[3-(4-Cyanophenoxy)propyl]-7-(2-hydroxyethyl)-3,7-diazabicyclo-[3.3.1]nonane-3-carboxamide

The title compound was prepared in 11% yield according to the procedure described in Example 14(c) above, using 3,7-diazabicyclo[3.3.1]nonane-3-ethanol (from step (e) above) and 4-(3-aminopropoxy)benzonitrile (from step (c) above) in place of 3-[3-(2-propyl-1,3-dioxolan-2-yl)propyl]-3,7-diazabicyclo[3.3.1]nonane and 4-(2-aminoethyl)benzonitrile, respectively.

¹³C NMR (CDCl₃): δ 162.04, 158.99, 133.66, 118.99, 115.03, 103.35, 66.55, 60.24, 57.87, 57.18, 50.02, 48.63, 37.93, 31.81, 29.26, 28.96

### Example 19

### N-{[7-[3-(4-Cyanophenoxy)-2-hydroxypropyl]-3,7-diazabicyclo[3.3.1]non-3-yl]carbonyl}-4-methylbenzenesulfonamide

A solution of 4-[3-(3,7-diazabicyclo[3.3.1]non-3-yl)-2-hydroxypropoxy]-benzonitrile (200 mg, 0.66 mmol, see Example G above) in chloroform (20 mL) was treated with a solution of p-toluenesulfonyl isocyanate (110 μL of 96% purity, 0.136 g, 0.69 mmol in chloroform (4 mL), added dropwise. A white precipitate immediately formed and the mixture was then concentrated in vacuo. The crude product so obtained was subjected to chromatography on silica gel, eluting with hexane:ethyl acetate:methanolic ammonia (75:75:50) to give the title compound in 53% yield.

¹³C NMR (CDCl₃): δ 15.77, 29.18, 32.37, 36.13, 48.72, 52.27, 56.32, 109.83, 113.13, 118.27, 118.93, 120.10, 127.80, 131.39, 132.46, 132.73, 134.62, 138.75, 159.14, 167.09

### Example 20

### N-Allyl-7-[3-(4-cyanophenoxy)-2-hydroxypropyl]-3,7-diazabicyclo[3.3.1]-nonane-3-carboxamide

A mixture of allylamine (125 μL, 1.66 mmol) and 1,1′-carbonyl-diimidazole (269 mg, 1.66 mmol) in THF (10 mL) was stirred at rt for 40 min. The mixture was then treated with a solution of 4-[3-(3,7-diazabicyclo[3.3.1]non-3-yl)-2-hydroxypropoxy]benzonitrile (see Example G above) in THF (5 mL), and stirring continued overnight. The mixture was concentrated in vacuo and the resulting residue purified by chromatography on silica gel, eluting with hexane:methanolic ammonia (1:1) to give the title compound in 57% yield.

¹³C NMR (MeOD): δ 29.37, 30.79, 41.95, 42.91, 58.91, 59.55, 61.12, 66.52, 70.75, 103.31, 113.81, 115.39, 118.72, 133.73, 135.57, 136.06, 158.93, 162.67

### Example 21

### 7-[3-(4-Cyanophenoxy)-2-hydroxypropyl]-N-[2-(2-thienyl)ethyl]-3,7-diazabicyclo[3.3.1]nonane-3-carboxamide

The title compound was prepared in 83% yield according to the procedure described in Example 19 above, using 2-(2-isocyanatoethyl)thiophene in place of p-toluenesulfonyl isocyanate.

¹³C NMR (CDCl₃): δ 29.19, 29.50, 30.59, 32.11, 42.26, 47.94, 49.37, 56.23, 60.47, 61.95, 65.32, 70.74, 103.88, 115.36, 119.52, 123.69, 125.25, 127.04, 133.90, 142.19, 158.74, 162.22

### Example 22

### 7-[3-(4-Cyanophenoxy)-2-hydroxypropyl]-N-[3-(ethylamino)-3-oxopropyl]-3,7-diazabicyclo[3.3.1]nonane-3-carboxamide

(a) Ethyl 3-[({7-[3-(4-cyanophenoxy)-2-hydroxypropyl]-3,7-diazabicyclo-[3.3.1]non-3-yl}carbonyl)amino]propanoate

The sub-title compound was prepared in 90% yield according to the procedure described in Example 12(a) above, using ethyl 3-isocyanatopropanoate in place of ethyl 2-isocyanatoacetate.

(b) 7-[3-(4-Cyanophenoxy)-2-hydroxypropyl]-N-[3-(ethylamino)-3-oxo-propyl]-3,7-diazabicyclo[3.3.1]nonane-3-carboxamide

The title compound was prepared in 22% yield according to the procedure described in Example 12(b) above, using ethyl 3-[({7-[3-(4-cyanophenoxy)-2-hydroxypropyl]-3,7-diazabicyclo[3.3.1]non-3-yl}-carbonyl)amino]propanoate (from step (a) above) and ethylamine in place of ethyl 2-[({7-[3-(4-cyanophenoxy)-2-hydroxypropyl]-3,7-diazabicyclo-[3.3.1]non-3-yl}carbonyl)amino]acetate and propylamine, respectively.

¹³C NMR (CDCl₃): δ 172.46, 162.17, 158.89, 133.96, 119.14, 115.37, 104.16, 65.27, 61.73, 60.58, 56.97, 49.23, 47.89, 37.51, 36.60, 34.26, 32.00, 29.54, 29.16, 14.87

### Example 23

### N-(1-Cyanoethyl)-7-[3-(4-cyanophenoxy)-2-hydroxypropyl]-3,7-diaza-bicyclo[3.3.1]nonane-3-carboxamide

(a) 2-Aminopropanenitrile

Lactonitrile (28 g, 375 mmol) was added to liquid ammonia at −78° C. in a reaction tube. The tube was sealed and the mixture was stirred overnight at rt. The ammonia was removed by evaporation and the crude material was used directly in the next step without any further purification.

(b) N-(1-Cyanoethyl)-7-[3-(4-cyanophenoxy)-2-hydroxypropyl]-3,7-diaza-bicyclo[3.3.1]nonane-3-carboxamide

A mixture of 2-aminopropanenitrile (250 mg, 3.58 mmol, from step (a) above) and N-ethyl di-iso-propylamine (0.67 mL, 0.50 g, 3.84 mmol) in DCM (9 mL) was added (by syringe pump), over the course of 1 hour, to a solution of triphosgene (352 mg, 1.19 mmol) in DCM (7 mL). The resulting mixture was stirred for 1 h at rt before a mixture of 4-[3-(3,7-diazabicyclo[3.3.1]non-3-yl)-2-hydroxypropoxy]benzonitrile (1.08 g, 3.58 mmol, see Example G above) and N-ethyl di-iso-propylamine (0.67 mL, 0.50 g, 3.84 mmol) in DCM (14 mL) was added. Stirring was continued for a further 20 min, before the solution was concentrated in vacuo and the resulting residue purified by flash chromatography, eluting with dichloromethane:methanol (95:5), to give the title compound in 65% yield.

¹³C NMR (CDCl₃): δ 20.02, 20.16, 29.11, 29.32, 29.46, 31.91, 37.83, 37.89, 48.23, 48.47, 49.36, 49.61, 56.95, 60.26, 60.51, 61.58, 62.077, 65.43, 70.69, 104.06, 115.40, 119.27, 120.77, 133.96, 157.08, 162.21

### Example 24

### 7-[3-(4-Cyanophenoxy)-2-hydroxypropyl]-N-(2,2,2-trifluoroethyl)-3,7-diazabicyclo[3.3.1]nonane-3-carboxamide

The title compound was prepared in 46% yield according to the procedure described in Example 23(b) above, using 2,2,2-trifluoroethylamine in place of 2-aminopropanenitrile.

¹³C NMR (CDCl₃): δ 29.11, 29.42, 31.79, 42.17, 42.51, 48.36, 49.58, 57.09, 60.45, 61.77, 65.39, 70.76, 104.08, 115.39, 119.23, 123.28, 126.05, 133.93, 157.76, 162.21

### Example 25

### 7-[3-(4-Cyanophenoxy)-2-hydroxypropyl]-N-[2-oxo-2-(1-piperidinyl)-ethyl]-3,7-diazabicyclo[3.3.1]nonane-3-carboxamide

The title compound was prepared in 49% yield according to the procedure described in Example 12(b) above, using piperidine in place of propylamine.

¹³C NMR (CDCl₃): δ 24.33, 25.41, 26.06, 28.74, 29.29, 29.44, 32.13, 42.67, 43.10, 45.30, 47.99, 48.09, 49.14, 49.28, 57.18, 60.42, 61.90, 65.55, 70.77, 94.22, 103.89, 115.24, 115.43, 119.24, 133.74, 134.02, 158.49, 162.20, 167.42

### Example 26

### N-(1,3-Benzodioxol-5-yl)-7-[3-(4-cyanophenoxy)-2-hydroxypropyl]-3,7-diazabicyclo[3.3.1]nonane-3-carboxamide

The title compound was prepared in 33% yield according to the procedure described in Example 23(b) above, using 1,3-benzodioxol-5-amine in place of 2-aminopropanenitrile.

¹³C NMR (CDCl₃): δ 162.22, 156.51, 147.47, 143.20, 133.98, 133.83, 119.41, 115.40, 113.68, 107.68, 103.83, 103.59, 100.96, 70.70, 65.98, 61.34, 60.34, 57.87, 49.17, 48.13, 31.52, 29.41, 29.11

### Example 27

### 7-[3-(4-Cyanoanilino)propyl]-N-[2-oxo-2-(propylamino)ethyl]-3,7-diazabicyclo[3.3.1]nonane-3-carboxamide

(a) 4-[(3-Hydroxypropyl)amino]benzonitrile

A mixture of 4-fluorobenzonitrile (12.0 g, 99.1 mmol) and 3-amino-1-propanol (59.6 g, 793 mmol) was stirred at 80° C. under an inert atmosphere for 3 hours before water (150 mL) was added. The mixture was allowed to cool to rt, and was then extracted with diethyl ether. The organic layer was separated, dried (Na₂SO₄), filtered and concentrated in vacuo to yield 17 g (97%) of the title compound as a oil that crystallised upon standing.

(b) 3-(4-Cyanoanilino)propyl 4-methylbenzenesulfonate

A cooled (0° C.) solution of 4-[(3-hydroxypropyl)amino]benzonitrile (17 g, 96.5 mmol, from step (a) above) in dry MeCN (195 mL) was treated with triethylamine (9.8 g, 96.5 mmol) and then p-toluenesulfonyl chloride (20.2 g, 106 mmol). The mixture was stirred at 0° C. for 90 minutes before being concentrated in vacuo. Water (200 mL) was added to the residue, and the aqueous solution was extracted with DCM. The organic phase was dried (Na₂SO₄), filtered and concentrated in vacuo. The resulting residue was purified by crystallisation from iso-propanol to yield 24.6 g (77%) of the sub-title compound.

(c) Ethyl 2-{[(7-benzyl-3,7-diazabicyclo[3.3.1]non-3-yl)carbonyl]amino}-acetate

The sub-title compound was prepared in 99% yield according to the procedure described in Example 5(a) above, using 4-[3-(3,7-diazabicyclo[3.3.1]non-3-yl)-2-hydroxypropoxy]benzonitrile (see Example G above) and ethyl 2-isocyanatoacetate in place of 3-benzyl-3,7-diazabicyclo[3.3.1]nonane and iso-propyl isocyanate, respectively.

(d) 7-Benzyl-N-[2-oxo-2-(propylamino)ethyl]-3,7-diazabicyclo[3.3.1]-nonane-3-carboxamide

The sub-title compound was prepared in 88% yield according to the procedure described in Example 12(b) above, using ethyl 2-{[(7-benzyl-3,7-diazabicyclo[3.3.1]non-3-yl)carbonyl]amino}acetate (from step (c) above) in place of ethyl 2-[({7-[3-(4-cyanophenoxy)-2-hydroxypropyl]-3,7-diazabicyclo[3.3.1]non-3-yl}carbonyl)amino]acetate.

(e) N-[2-Oxo-2-(propylamino)ethyl]-3,7-diazabicyclo[3.3.1]nonane-3-carboxamide

The title compound was prepared according to the procedure described in Example 5(b) above, using 7-benzyl-N-[2-oxo-2-(propylamino)ethyl]-3,7-diazabicyclo[3.3.1]nonane-3-carboxamide (from step (d) above) in place of 7-benzyl-N-iso-propyl-3,7-diazabicyclo[3.3.1]nonane-3-carboxamide.

(f) 7-[3-(4-Cyanoanilino)propyl]-N-[2-oxo-2-(propylamino)ethyl]-3,7-diazabicyclo[3.3.1]nonane-3-carboxamide

A mixture of N-[2-oxo-2-(propylamino)ethyl]-3,7-diazabicyclo[3.3.1]-nonane-3-carboxamide (3.35 g, 12.5 mmol, from step (e) above), K₂CO₃ (6.9 g, 50 mmol) and sodium iodide (0.19 g, 1.25 mmol) in acetonitrile (600 mL) was treated with 3-(4-cyanoanilino)propyl 4-methyl-benzenesulfonate (4.2 g, 12.7 mmol, from step (b) above) and stirred under reflux for 5 h, followed by a further 21 h at rt. The mixture was filtered, concentrated in vacuo and the crude product so obtained was diluted with water. The aqueous solution was extracted with DCM, which organic layer was separated, dried and concentrated in vacuo. The crude product so obtained was purified by chromatography on silica gel, eluting with DCM:MeOH (95:5) to yield 3.08 g (58%) of the title compound.

¹³C NMR (CDCl₃): δ 11.49, 22.85, 25.11, 29.09, 31.03, 40.78, 41.40, 44.80, 48.41, 56.22, 59.32, 97.43, 111.99, 120.97, 133.74, 151.98, 157.92, 170.37

### Example 28

### 7-{2-[2-(4-Cyanophenoxy)ethoxy]ethyl}-N-ethyl-3,7-diazabicyclo-[3.3.1]nonane-3-carboxamide

(a) 4-[2-(2-Hydroxyethoxy)ethoxy]benzonitrile

A mixture of p-cyanophenol (11.9 g, 100 mmol), K₂CO₃ (15 g, 110 mmol) and chloroethoxyethanol (12.4 g 100 mmol) in CH₃CN was refluxed for 24 h, then stirred at rt for a further 2 days. The reaction mixture was filtered and concentrated in vacuo to give a crude product which was purified by chromatography on silica gel (hexane:ethyl acetate (1:1) eluant). This gave 10 g (50%) of the sub-title compound.

(b) 2-[2-(4-Cyanophenoxy)ethoxy]ethyl methanesulfonate

Methanesulfonyl chloride (3.0 g, 26 mmol) was added dropwise to a cooled (−5 ° C.) mixture of 4-[2-(2-hydroxyethoxy)ethoxy]benzonitrile (5.0 g, 24 mmol, from step (a) above) and triethylamine (4 mL, 2.9 g, 29 mmol) in DCM (50 mL). After addition was complete, the reaction was allowed to warm to rt over a period of 2 h. The reaction mixture was then washed twice with water, the organic layer separated, dried (Na₂CO₃) and concentrated in vacuo to yield 7 g (100%) of the sub-title compound.

(c) 7-{2-[2-(4-Cyanophenoxy)ethoxy]ethyl}-N-ethyl-3,7-diazabicyclo-[3.3.1]nonane-3-carboxamide

A mixture of 2-[2-(4-cyanophenoxy)ethoxy]ethyl methanesulfonate (2 g, 7.0 mmol, from step (b) above), N-ethyl-3,7-diazabicyclo[3.3.1]nonane-3-carboxamide (1.4 g, 7.0 mmol, see Example 6(b) above) and K₂CO₃ (1.5 g, 10.5 mmol) in MeCN (50 mL) was stirred under reflux overnight. The reaction mixture was then concentrated in vacuo and the resulting residue purified by flash chromatography on silica gel, eluting with dichloromethane:methanol (9:1) to yield 0.8 g (30%) of the title compound.

¹³C NMR (CDCl₃): δ 162.18, 133.92, 119.24, 115.34, 103.92, 69.07, 67.86, 59.52, 58.42, 48.18, 35.68, 30.25, 28.81, 15.69

### Example 29

### 7-[4-(4-Cyanophenyl)-4-(3,4-dimethoxyphenoxy)butyl]-N-ethyl-3,7-diaza-bicyclo[3.3.1]nonane-3-carboxamide

(a) 4-[1-(3,4-Dimethoxyphenoxy)-3-butenyl]benzonitrile

A cooled (0° C.) mixture of 4-(1-hydroxy-3-butenyl)benzonitrile (14.6 g, 84.3 mmol) and 3,4-dimethoxyphenol (19.5 g, 125.4 mmol) in toluene (500 mL) was treated with tributylphosphine (32.14 mL of 97% purity, 25.6 g, 126.4 mmol), followed by 1,1′-(azodicarbonyl)dipiperidine (31.8 g, 126.4 mmol). After addition was complete, the reaction mixture thickened and the temperature rose to 15° C. Additional toluene was added (500 mL), and the mixture stirred at rt overnight. The precipitate of tributylphosphine oxide was then removed by filtration and the filtrate concentrated in vacuo to give 65.8 g of crude product. This was purified by chromatography on silica gel, eluting with toluene:methanol (98:2) to yield 17.9 g of the sub-title compound.

(b) 4-[1-(3,4-Dimethoxyphenoxy)-4-hydroxybutyl]benzonitrile

Borane-methyl sulfide complex (2 M in ether, 11 mL, 22 mmol) was added dropwise to a cooled (−5° C.) solution of 4-[1-(3,4-dimethoxy-phenoxy)-3-butenyl]benzonitrile (17.6 g, 56.8 mmol, from step (a) above) in dry THF (15 mL) over a period of 15 minutes (during which time the reaction temperature rose to 0° C.). The resulting mixture was stirred at between 0 and 10° C. for 1.5 h, before being allowed to warm to rt. Stirring was continued for a further 3.5 h at this temperature before water (22 mL) and sodium perborate tetrahydrate (11 g, 66 mmol) were added. The biphasic mixture was stirred for 2 h at rt before the water layer was separated and extracted with ether. The combined organic layers were washed with brine, dried and concentrated in vacuo. The resulting residue was purified by chromatography on silica gel, eluting with iso-propanol:ethyl acetate:heptane (5:25:70) to yield 14.5 g (77%) of the sub-title compound.

(c) 4-(4-Cyanophenyl)-4-(3,4-dimethoxyphenoxy)butyl methanesulfonate

A solution of methanesulfonyl chloride (3.4 mL, 5.0 g, 44 mmol) in DCM (15 mL) was added slowly to a cooled (−5° C.) mixture of 4-[1-(3,4-dimethoxyphenoxy)-4-hydroxybutyl]benzonitrile (11 g, 34 mmol, from step (b) above) and triethylamine (7 mL, 5.2 g, 50.6 mmol) in DCM (50 mL), during which addition the temperature did not rise above 2° C. Stirring was continued at between 0 and 5° C. for a further 2 h before water was added. The resulting organic layer was separated, and washed with water, separated again and then dried to give the sub-title compound in 100% yield.

(d) tert-Butyl 7-[4-(4-cyanophenyl)-4-(3,4-dimethoxyphenoxy)butyl]-3,7-diazabicyclo[3.3.1]nonane-3-carboxylate

A mixture of 4-(4-cyanophenyl)-4-(3,4-dimethoxyphenoxy)butyl methane-sulfonate (522 mg, 1.29 mmol, from step (c) above), tert-butyl 3,7-diazabicyclo[3.3.1]nonane-3-carboxylate (307 mg, 1.356 mmol, see Example F above) and K₂CO₃ (216 mg, 1.56 mmol) in chloroform:acetonitrile (10 mL of 1:1) was stirred at 70° C. for 23 h. The reaction mixture was filtered and the filtrate concentrated in vacuo to give 708 mg of crude product. This was purified by flash chromatography, eluting with a gradient of toluene:methanol (97:3 to 10:1), to yield 607 mg (88%) of the sub-title compound.

(e) 4-[4-(3,7-Diazabicyclo[3.3.1]non-3-yl)-1-(3,4-dimethoxyphenoxy)-butyl]benzonitrile

A cooled (0° C.) solution of tert-butyl 7-[4-(4-cyanophenyl)-4-(3,4-dimethoxyphenoxy)butyl]-3,7-diazabicyclo[3.3.1]nonane-3-carboxylate (1.92 g, 3.6 mmol, from step (d) above) in ethyl acetate (20 mL) was treated with HCl-saturated ethyl acetate (30 mL). The resulting mixture was stirred for 2 h at between 0 and 5° C. before being concentrated in vacuo. The resulting residue was dissolved in acetonitrile (50 mL) and treated with K₂CO₃ (3.5 g, 25.2 mmol) and water (2.25 mL). This mixture was stirred for 3 h at rt and the solids removed by filtration, before the solvent was removed in vacuo (with toluene added to effect azeotropic removal of water) to give 1.5 g of the sub-title compound.

(f) 7-[4-(4-Cyanophenyl)-4-(3,4-dimethoxyphenoxy)butyl]-N-ethyl-3,7-diazabicyclo[3.3.1]nonane-3-carboxamide

A solution of 4-[4-(3,7-diazabicyclo[3.3.1]non-3-yl)-1-(3,4-dimethoxy-phenoxy)butyl]benzonitrile (109 mg, 0.25 mmol, from step (e) above), in CHCl₃ (1.43 mL) was treated with a solution of ethyl isocyanate (18.6 μL, 16.8 mg, 0.237 mmol) in MeCN (0.5 mL). The resulting mixture was stirred for 30 h. at rt. The solution was then loaded onto an ion-exchange solid phase extraction plug (SiO₂, 0.5 g from ISOLUTE). The plug was washed with CHCl₃ (2.5 mL) and the product then eluted with MeCN (3×2.5 mL). This gave the title compound (93 mg, 73%) in a purity better than 90% (as determined by HPLC: UV at 254 nm and ELS detection).

MS (ES) m/z=507 (M+1)⁺, 505 (M−1)⁻

### Example 30

### 7-(3-{4-Cyano-2-[(cyclopropylamino)carbonyl]phenox}-2-hydroxy-propyl)-N-phenyl-3,7-diazabicyclo[3.3.1]nonane-3-carboxamide

(a) 5-Cyano-N-cyclopropyl-2-[2-oxiranylmethoxy]benzamide

The sub-title compound was prepared according to the method described in Example 7(b) above using 2-oxiranylmethyl 3-nitrobenzenesulfonate (prepared analogously to the method described in Example B above).

(b) 7-Benzyl-N-phenyl-3,7-diazabicyclo[3.3.1]nonane-3-carboxamide

A cooled (0° C.) solution of 3-benzyl-3,7-diazabicyclo[3.3.1]nonane (10 g, 46 mmol, see Example E above) in DCM (100 mL) was treated with phenyl isocyanate (4.9 mL, 45 mmol). The mixture was stirred at rt for 30 min. The product formed as white crystals, which were removed by filtration to give 10 g (66%) of the sub-title compound.

(c) N-Phenyl-3,7-diazabicyclo[3.3.1]nonane-3-carboxamide

A solution of 7-benzyl-N-phenyl-3,7-diazabicyclo[3.3.1]nonane-3-carboxamide (10 g, 29.8 mmol, from step (b) above) in ethanol (100 mL) was subjected to hydrogenation, over 10% Pd/C and at ambient pressure, overnight. The catalyst was removed through a pad of Celite® and the residue was concentrated in vacuo to give the sub-title compound in quantitative yield.

(d) 7-(3-{4-Cyano-2-[(cyclopropylamino)carbonyl]phenoxy}-2-hydroxy-propyl)-N-phenyl-3,7-diazabicyclo[3.3.1]nonane-3-carboxamide

A mixture of 5-cyano-N-cyclopropyl-2-[2-oxiranylmethoxy]benzamide (0.8 g, 3.1 mmol, from step (a) above) and N-phenyl-3,7-diaza-bicyclo[3.3.1]nonane-3-carboxamide (0.9 g, 3.6 mmol, from step (c) above) in iso-propanol:H₂O (10 mL of 9:1) was refluxed for 180 min. before dichloromethane was added and the solvent removed in vacuo. Purification of the resulting residue by flash chromatography, eluting with DCM:MeOH (9:1), gave 1 g (64%) of the title compound.

¹³C NMR (CDCl₃): δ 6.33, 6.56, 23.23, 29.18, 29.51, 31.66, 48.27, 49.60, 53.44, 57.94, 60.51, 65.74, 71.28, 104.93, 113.46, 118.45, 119.54, 119.65, 122.88, 123.27, 128.84, 136.07, 156.44, 159.69, 164.53

### Example 31

### N-(4-Cyanophenyl)-7-[3-(ethanesulfonyl)propyl]-3,7-diazabicyclo-[3.3.1]nonane-3-carboxamide

(a) 3-(Ethanesulfonyl)propyl 4-methylbenzenesulfonate

Triethylamine (13.36 g, 132 mmol) was added dropwise to a mixture of 3-(ethanesulfonyl)-1-propanol (13.4 g, 88 mmol, Martin-Smith et al., J. Pharm. Pharmacol., 19, (1967) 649) and p-toluenesulfonyl chloride (16.78 g, 88 mmol) in DCM (150 mL), resulting in a mildly exothermic reaction. After addition was complete, the reaction mixture was washed twice with aqueous ammonium chloride solution, the organic layer was then separated, dried, and concentrated in vacuo. The resulting residue was recrystallised from diethyl ether/DCM to give 17.9 g (65%) of the sub-title compound.

(b) tert-Butyl 7-[(4-cyanoanilino)carbonyl]-3,7-diazabicyclo[3.3.1]nonane-3-carboxylate

A suspension of tert-butyl-3,7-diazabicyclo[3.3.1]nonane-3-carboxylate (2.0 g, 8.8 mmol, see Example F above) in chloroform (15 mL) was treated with 4-isocyanatobenzonitrile (1.53 g, 10.6 mmol). The mixture was stirred at rt for 1.5 h, at which time some solid particles were observed in the mixture. An additional 10 mL of chloroform was added in order to dissolve the particles. Mass spectroscopic analysis of the mixture indicated that the starting materials had been consumed, and so the solvent was removed in vacuo. The resulting residue was purified by flash chromatography, eluting with a gradient of DCM:MeCN (5:1 to 2:1) to yield 2.31 g (71%) of the sub-title compound.

(c) N-(4-Cyanophenyl)-3,7-diazabicyclo[3.3.1]nonane-3-carboxamide

A cooled (0° C.) solution of tert-butyl 7-[(4-cyanoanilino)carbonyl]-3,7-diazabicyclo[3.3.1]nonane-3-carboxylate (2.2 g 5.94 mmol, from step (b) above) in ethyl acetate (40 mL) was treated with HCl-saturated ethyl acetate (65 mL) over the course of 30 minutes. The resulting mixture was stirred at rt for a further 4 h before being concentrated in vacuo to give 1.8 g (99%) of the hydrochloride salt of the sub-title compound.

(d) N-(4-Cyanophenyl)-7-[3-(ethanesulfonyl)propyl]-3,7-diazabicyclo-[3.3.1]nonane-3-carboxamide

A mixture of N-(4-cyanophenyl)-3,7-diazabicyclo[3.3.1]nonane-3-carboxamide (67.6 mg, 0.25 mmol, from step (c) above) and K₂CO₃ (80 mg, 0.57 mmol) in DMF (0.5 mL) was treated with a solution of 3-(ethanesulfonyl)propyl 4-methylbenzenesulfonate (153 mg, 0.50 mmol, from step (a) above), in MeCN (1.0 mL). The resulting suspension was stirred for 5 days at 50° C. before being cooled and filtered. The filtrate was then added to a ion-exchange solid phase extraction plug (CBA, 2 g from ISOLUTE). After 1 h the plug was washed with CHCl₃ (3×2.5 mL) and the product eluted with CHCl₃:MeOH:Et₃N (8:1:1) to give the title compound (63.6 mg, 63%) in a purity better than 90% (as determined by HPLC: UV at 254 nm and ELS detection).

MS (ES): m/z=405 (M+1)⁺, m/z=403 (M−1)⁻

### Example 32

### 7-{3-[(2-Cyano-1H-indol-4-yl)oxy]-2-hydroxypropyl}-N-phenyl-3,7-diazabicyclo[3.3.1]nonane-3-carboxamide

A mixture of 4-(2-oxiranylmethoxy)-1H-indole-2-carbonitrile (1.0 g, 4.7 mmol, Pitha et al., J. Med. Chem., 30 (1987) 612) and N-phenyl-3,7-diazabicyclo[3.3.1]nonane-3-carboxamide (1.4 g, 5.5 mmol, see Example 30(d) above) in iso-propanol:H₂O (10 mL of 9:1) was stirred under reflux for 3 h before being concentrated in vacuo. The resulting residue was purified by column chromatography on silica gel, eluting with a gradient of DCM:MeOH (99:1 to 97:3), to yield 0.8 g (37%) of the title compound.

¹³C NMR (CDCl₃): δ 29.03, 29.39, 31.27, 48.37, 49.31, 57.89, 60.42, 61.41, 66.07, 70.04, 100.72, 104.39, 105.13, 111.31, 114.95, 117.66, 120.18, 120.30, 123.00, 126.54, 128.84, 138.39, 139.16, 152.55, 156.29

### Example 33

### 7-[(7-Cyano-2,3-dihydro-1,4-benzodioxin-2-yl)methyl]-N-ethyl-3,7-diazabicyclo[3.3.1]nonane-3-carboxamide

(a) 5-Bromo-2-(3-chloro-2-hydroxypropoxy)benzaldehyde

A mixture of 5-bromo-2-hydroxy benzaldehyde (20.1 g, 0.1 mol), epichlorohydrin (25 mL, 0.32 mol) and 6 drops of piperidine was stirred under reflux for 6 h before being concentrated in vacuo. The resulting residue was dissolved in chloroform (25 mL) and treated with concentrated HCl (10 mL). The resulting mixture was stirred for 3 h at rt before the organic layer was washed with water, separated, dried and concentrated in vacuo to yield 28.2 g (96%) the sub-title compound. This was used directly in the next step without any further purification.

(b) 5-Bromo-2-(3-chloro-2-hydroxypropoxy)phenyl formate

A solution of 5-bromo-2-(3-chloro-2-hydroxypropoxy)benzaldehyde (28.2 g, 96 mmol, from step (a) above) in DCM (200 mL) was treated with 3-chloroperoxybenzoic acid (25 g of 70-75% purity, approximately 100 mmol). The resulting exothermic reaction caused the mixture to reflux for 20 min. Stirring was continued for a further 3 days before the mixture was filtered (to remove precipitated 3-chlorobenzoic acid). The filtrate was washed with K₂CO₃-solution and water, dried and concentrated in vacuo to yield 26.1 g of sub-title compound. This was used directly in the next step without any further purification.

(c) (7-Bromo-2,3-dihydro-1,4-benzodioxin-2-yl)methanol

A solution of 5-bromo-2-(3-chloro-2-hydroxypropoxy)phenyl formate (26.1 g, 84 mmol, from step (b) above) in ethanol (100 mL) was treated with a solution of potassium hydroxide (6.1 g of 85% purity, approximately 92 mmol) in water (10 mL). The resulting mixture was refluxed for 1.5 h before being filtered and concentrated in vacuo. The resulting residue was dissolved in ethyl acetate and washed with brine. The organic layer was separated, dried and concentrated in vacuo to give 28.8 g of crude product. This was purified by column chromatography on silica gel, eluting with diethyl ether:hexane (70:30), to yield 10.0 g (49.1%) of the sub-title compound.

(d) 3-(Hydroxymethyl)-2,3-dihydro-1,4-benzodioxin-6-carbonitrile

A mixture of (7-bromo-2,3-dihydro-1,4-benzodioxin-2-yl)methanol (10.0 g, 41.2 mmol, from step (c) above) and CuCN (4. 0 g, 45.3 mmol) in DMF (10 mL, dried over molecular sieves) was stirred at 170° C. for 4.5 h. The reaction mixture was poured into a warm aqueous solution of sodium cyanide (8.10 g, 165 mmol of NaCN in 25 mL H₂O). The resulting mixture was extracted with toluene and DCM. The combined organic layers were washed with water and then brine, dried and concentrated in vacuo. The residue so obtained was crystallised from toluene and DCM to yield 2.8 g (35%) of the sub-title compound.

(e) (7-Cyano-2,3-dihydro-1,4-benzodioxin-2-yl)methyl methanesulfonate

A solution of methanesulfonyl chloride (1.81 g, 15.8 mmol) in dichloromethane (5 mL) was added dropwise to a cooled (0° C.) mixture of 3-(hydroxymethyl)-2,3-dihydro-1,4-benzodioxin-6-carbonitrile (2.75 g, 14.4 mmol, from step (d) above) and pyridine (1.26 g, 16 mmol) in DCM (25 mL). After addition was complete, the mixture was stirred at 0° C. for 1 h, and then at rt overnight. TLC analysis indicated incomplete reaction after this time, and so further portions of methanesulfonyl chloride (0.4 g, 3.5 mmol) and pyridine (0.5 mL, 0.49 g, 6.2 mmol) were added. The mixture was refluxed for 3.5 h before being washed twice with saturated Na₂CO₃ solution, dried and concentrated in vacuo. The crude product (4.5 g) so obtained was purified by flash chromatography, eluting with DCM, to give 3.5 g of the sub-title compound, which crystallised on standing.

(f) 7-[(7-Cyano-2,3-dihydro-1,4-benzodioxan-2-yl)methyl]-N-ethyl-3,7-diazabicyclo[3.3.1]nonane-3-carboxamide

A mixture of (7-cyano-2,3-dihydro-1,4-benzodioxin-2-yl)methyl methane-sulfonate (150 mg, 0.9 mmol, from step (e) above), N-ethyl-3,7-diaza-bicyclo[3.3.1]nonane-3-carboxamide (186 mg, 0.94 mmol, see Example 6(b) above), K₂CO₃ (265 mg, 2.0 mmol) and NaI (14 mg, 0.09 mmol) in CH₃CN was refluxed for 20 h. The solvent was removed in vacuo and the resulting residue treated with DCM and water. The organic layer was separated, dried (Na₂SO₄) and concentrated in vacuo. The resulting residue was purified by flash chromatography, eluting with DCM:MeOH (95:5) to yield 113.2 mg (34%) of the title compound.

¹³C NMR (CDCl₃): δ 15.61, 29.19, 30.72, 35.72, 47.78, 58.34, 59.02, 60.64, 67.01, 71.38, 71.49, 71.60, 104.10, 120.76, 120.89, 125.39, 125.79, 143.50, 147.80, 157.46

### Example 34

### 7-{[(2S)-6-Cyano-4-(methanesulfonyl)-3,4-dihydro-2H-1,4-benzoxazin-2-yl]methyl}-N-ethyl-3,7-diazabicyclo[3.3.1]nonane-3-carboxamide

(a) (2R)-2-(Hydroxymethyl)-3,4-dihydro-2H-1,4-benzoxazine-6-carbonitrile

A mixture of 3-amino-4-hydroxybenzonitrile (25 g, 0.186 mol) and S-epichlorohydrin (10.7 g, 0.22 mol) in aqueous ethanol (500 mL of 99%) was stirred at 60° C. for 24 h. The mixture was concentrated in vacuo before ethanol (500 mL) was added, followed by K₂CO₃ (27 g, 0.195 mol). The resulting mixture was refluxed for 1 h before being filtered. The filtrate was concentrated in vacuo to give 61 g of a black oil. This was diluted with water (500 mL), and then extracted twice with DCM and ethyl acetate. The combined organic extracts were dried and concentrated in vacuo to yield 20 g (57%) of the sub-title compound as yellow crystals.

(b) (2R)-6-Cyano-4-(methanesulfonyl)-3,4-dihydro-2H-1,4-benzoxazin-2-yl]methyl methanesulfonate

Methanesulfonyl chloride (45 g, 0.395 mol) was added dropwise to a cooled (0° C.) mixture of (2R)-2-(hydroxymethyl)-3,4-dihydro-2H-1,4-benzoxazine-6-carbonitrile (30 g, 0.158 mol, from step (a) above) and pyridine (200 mL, excess). The mixture was stirred at rt overnight before being concentrated in vacuo. The resulting residue was treated with water and crystals of the product were isolated by filtration. These were recrystallised from MeCN to give 29 g of pure material. The mother liquor was concentrated in vacuo to give a residue which was crystallised from chloroform to give a further crop (7.5 g) of product. The total yield of the sub-title compound was 36.5 g (67%).

(c) 7-{[(2S)-6-Cyano-4-(methanesulfonyl)-3,4-dihydro-2H-1,4-benzoxazin-2-yl]methyl}-N-ethyl-3,7-diazabicyclo[3.3.1]nonane-3-carboxamide

A solution of (2R)-6-cyano-4-(methanesulfonyl)-3,4-dihydro-2H-1,4-benzoxazin-2-yl]methyl methanesulfonate (1 g, 2.89 mmol, from step (b) above) in MeCN (5 mL) was treated with triethylamine (8 mL, 5.8 g, 57.4 mmol), followed by N-ethyl-3,7-diazabicyclo[3.3.1]nonane-3-carboxamide (0.85 g, 4.33 mmol, see Example 6(b) above). The resulting mixture was stirred at 70° C. for 5 h, and then at rt overnight. The mixture was concentrated in vacuo and purified by acid/base extraction, followed by flash chromatography, eluting with DCM:MeOH, to yield 100 mg (14%) of the title compound.

¹³C NMR (CDCl₃): δ 15.63, 28.87, 29.09, 30.48, 35.73, 39.50, 45.96, 47.65, 48.11, 59.03, 59.19, 60.59, 73.40, 104.15, 118.72, 119.90, 124.92, 126.51, 128.92, 150.04, 157.74

### Example 35

### 7-[2-({2-[4,5-Bis(4-cyanophenyl)-1H-pyrazol-1-yl]acetyl}amino)ethyl]-N-ethyl-3,7-diazabicyclo[3.3.1]nonane-3-carboxamide

(a) 4-[(E)-1-(4-Cyanobenzoyl)-2-(dimethylamino)ethenyl]benzonitrile

N,N-Dimethylformamide dimethylacetal (135.2 g, 0.29 mol) was added dropwise, under an inert atmosphere, to a heated (60° C.) solution of 4-[2-(4-cyanophenyl)acetyl]benzonitrile (60.2 g, 0.24 mol, Ashley et al., J. Chem. Soc. (1942) 103, 110) in 1,2-dimethoxyethane. The resulting mixture was then filtered and concentrated in vacuo to give a residue that was crystallised from MeOH. This gave 27.9 g (38%) of the sub-title compound.

(b) Ethyl 2-[4,5-bis(4-cyanophenyl)-1H-pyrazol-1-yl]acetate

A solution of 4-[(E)-1-(4-cyanobenzoyl)-2-(dimethylamino)ethenyl]-benzonitrile (6.2 g, 20 mmol from step (a) above) in aqueous ethanol (100 mL of 99%) was treated with ethyl 2-hydrazinoacetate hydrochloride (3.5 g, 22.6 mmol). The mixture was stirred at rt overnight before being concentrated in vacuo. The resulting residue was diluted with water, which aqueous mixture was extracted with DCM. The organic layer was then separated, dried and concentrated in vacuo to give a residue which was recrystallised from diethyl ether to yield 1.7 g (23.5%) of the sub-title compound.

(c) 2-[4 ,5-Bis(4-cyanophenyl)-1H-pyrazol-1-yl]-N-(2-hydroxyethyl)-acetamide

A mixture of ethyl 2-[4,5-bis(4-cyanophenyl)-1H-pyrazol-1-yl]acetate (3.9 g, 10.9 mmol, from step (b) above), 2-amino-1-ethanol (1.3 g, 21.8 mmol) and triethylamine (0.8 g, 76 mmol) was stirred at 100° C. overnight. Water and DCM were added, the product crystallised and was isolated by filtration to yield 3.53 g of sub-title compound.

(d) 2-[4,5-Bis(4-cyanophenyl)-1H-pyrazol-1-yl]-N-(2-bromoethyl)-acetamide

A mixture of 2-[4,5-bis(4-cyanophenyl)-1H-pyrazol-1-yl]-N-(2-hydroxy-ethyl)acetamide (0.7 g, 1.88 mmol, from step (c) above), N-bromo-succinimide (0.75 g, 5.64 mmol) and triphenylphosphine (2.22 g, 8.4 mmol) in DCM (100 mL) was stirred under reflux for 3 h. The reaction mixture was allowed to cool before being washed with water. The organic layer was separated, dried and concentrated in vacuo to give a residue that was purified by flash chromatography, eluting with diethyl ether:methanol (95:5), to yield 0.7 g sub-title compound contaminated with triphenylphosphine oxide. This product was used directly in the next step without any further purification.

(e) 7-[2-({2-[4,5-Bis(4-cyanophenyl)-1H-pyrazol-1-yl]acetyl}amino)ethyl]-N-ethyl-3,7-diazabicyclo[3.3.1]nonane-3-carboxamide

A mixture of 2-[4,5-bis(4-cyanophenyl)-1H-pyrazol-1-yl]-N-(2-bromo-ethyl)acetamide (0.7 g, 1.6 mmol, from step (d) above), N-ethyl-3,7-diazabicyclo[3.3.1]nonane-3-carboxamide (0.32 g, 1.6 mmol, see Example 6(b) above) and K₂CO₃ (0.55 g, 4 mmol) in acetonitrile (15 mL) was stirred under reflux overnight. Extraction with diethyl ether and water gave an organic layer that was separated, dried and concentrated in vacuo. The resulting residue was purified by chromatography on silica gel, eluting with diethyl ether: MeOH (95:5), to yield 0.27 g of the title compound.

¹³C NMR (CDCl₃): δ 15.77, 29.18, 32.37, 36.13, 48.72, 52.27, 56.32, 109.83, 113.13, 118.27, 118.93, 120.10, 127.80, 131.39, 132.46, 132.73, 134.62, 138.75, 159.14, 167.09

### Example 36

The following compounds (all of which are title compounds of this Example 36) were also prepared, using analogous methods to those described herein:

7-[3-(4-cyanophenoxy)-2-hydroxypropyl]-N-ethyl-3,7-diazabicyclo[3.3.1]-nonane-3-carboxamide;

7-[(2R)-3-(4-cyanophenoxy)-2-hydroxypropyl]-N-ethyl-3,7-diazabicyclo-[3.3.1]nonane-3-carboxamide;

7-[3-(4-cyanophenoxy)-2-hydroxypropyl]-N-propyl-3,7-diazabicyclo-[3.3.1]nonane-3-carboxamide;

7-[2-(4-cyanophenoxy)ethyl]-N-ethyl-3,7-diazabicyclo[3.3.1]nonane-3-carboxamide;

7-[3-(4-cyanophenoxy)-2-hydroxypropyl]-N-tetrahydro-2H-pyran-2-yl-3,7-diazabicyclo[3.3.1]nonane-3-carboxamide;

7-[3-(4-cyanophenoxy)-2-hydroxypropyl]-3,7-diazabicyclo[3.3.1]nonane-3-carboxamide;

7-(4-cyanophenethyl)-N-ethyl-3,7-diazabicyclo[3.3.1]nonane-3-carboxamide;

7-[(2S)-3-(4-cyanophenoxy)-2-hydroxypropyl]-N,N-dimethyl-3,7-diazabicyclo[3.3.1]nonane-3-carboxamide;

tert-butyl 2-(4-cyanophenoxy)-1-({7-[(ethylamino)carbonyl]-3,7-diazabi-cyclo[3.3.1]non-3-yl}methyl)ethylcarbamate;

7-(3-(4-cyanophenoxy)-2-{[(ethylamino)carbonyl]amino}propyl)-N-ethyl-3,7-diazabicyclo[3.3.1]nonane-3-carboxamide;

7-(3-(4-cyanophenoxy)-2- {[(ethylamino)carbonyl] amino}propyl)-N-ethyl-3,7-diazabicyclo[3.3.1]nonane-3-carboxamide;

7-(3-(4-cyanophenoxy)-2-{[(dimethylamino)carbonyl]amino}propyl)-N-ethyl-3,7-diazabicyclo[3.3.1]nonane-3-carboxamide;

methyl 2-(4-cyanophenoxy)-1-({7-[(ethylamino)carbonyl]-3,7-diazabi-cyclo[3.3.1]non-3-yl}methyl)ethylcarbamate;

7-[2-(acetylamino)-3-(4-cyanophenoxy)propyl]-N-ethyl-3,7-diazabicyclo-[3.3.1]nonane-3-carboxamide;

7-[3-(2,4-dicyanophenoxy)-2-hydroxypropyl]-N-ethyl-3,7-diazabicyclo-[3.3.1]nonane-3-carboxamide;

tert-butyl (1S)-2-(4-cyanophenoxy)-1-({7-[(ethylamino)carbonyl]-3,7-diazabicyclo[3.3.1]non-3-yl}methyl)ethylcarbamate;

7-[(2S)-2-[(aminocarbonyl)amino]-3-(4-cyanophenoxy)propyl]-N-ethyl-3,7-diazabicyclo[3.3.1]nonane-3-carboxamide;

tert-butyl (1R)-2-(4-cyanophenoxy)-1-({7-[(ethylamino)carbonyl]-3,7diazabicyclo[3.3.1]non-3-yl}methyl)ethylcarbamate;

N-acetyl-7-[(2R)-3-(4-cyanophenoxy)-2-hydroxypropyl]-3,7-diazabicyclo-[3.3.1]nonane-3-carboxamide;

N-acetyl-7-[(2S)-3-(4-cyanophenoxy)-2-hydroxypropyl]-3,7-diazabicyclo-[3.3.1]nonane-3-carboxamide;

7-[(2R)-3-(4-cyanophenoxy)-2-hydroxypropyl]-N-methyl-3,7-diazabicyclo[3.3.1]nonane-3-carboxamide;

7-[(2S)-3-(4-cyanophenoxy)-2-hydroxypropyl]-N-methyl-3,7-diazabicyclo-[3.3.1]nonane-3-carboxamide; 3.3.1]nonane-3-carboxamide;

7-[(2S)-3-(4-cyano-2-{[(2-cyanoethyl)amino]carbonyl}phenoxy)-2-hydroxypropyl]-N-ethyl-3,7-diazabicyclo[3.3.1]nonane-3-carboxamide;

methyl (1R)-2-(4-cyanophenoxy)-1-({7-[(ethylamino)carbonyl]-3,7-diazabicyclo[3.3.1]non-3-yl}methyl)ethylcarbamate;

7-[(2R)-3-(4-cyanophenoxy)-2-hydroxypropyl]-N-propyl-3,7-diazabicyclo-[3.3.1]nonane-3-carboxamide;

7-[(2S)-3-(4-cyanophenoxy)-2-hydroxypropyl]-N-propyl-3,7-diazabicyclo[3.3.1]nonane-3-carboxamide;

7-((2S)-3-{4-cyano-2-[(methylamino)carbonyl]phenoxy}-2-hydroxypropyl)-N-ethyl-3,7-diazabicyclo[3.3.1]nonane-3-carboxamide;

7-[(2S)-3-(4-cyanophenoxy)-2-hydroxypropyl]-N-propionyl-3,7-diazabicyclo[3.3.1]nonane-3-carboxamide;

7-[(2R)-3-(4-cyanophenoxy)-2-hydroxypropyl]-N-propionyl-3,7-diazabicyclo[3.3.1]nonane-3-carboxamide;

7-[2-(4-cyanophenyl)-2-hydroxyethyl]-N-ethyl-3,7-diazabicyclo[3.3.1]-nonane-3-carboxamide;

7-[(2S)-3-(4-cyanophenoxy)-2-hydroxypropyl]-N-(2-propynyl)-3,7-diazabicyclo[3.3.1]nonane-3-carboxamide;

7-(4-cyanophenethyl)-N-iso-propyl-3,7-diazabicyclo[3.3.1]nonane-3-carboxamide;

N-ethyl-7-[(2S)-2-hydroxy-3-(4-nitrophenoxy)propyl]-3,7-diazabicyclo-[3.3.1]nonane-3-carboxamide;

methyl (1S)-2-(4-cyanophenoxy)-1-({7-[(ethylamino)carbonyl]-3,7-diazabicyclo[3.3.1]non-3-yl}methyl)ethylcarbamate;

7-[(2S)-3-(4-cyanophenoxy)-2-hydroxypropyl]-N-(cyclopropylmethyl)-3,7-diazabicyclo[3.3.1]nonane-3-carboxamide;

N-(4-nitrophenyl)-7-(4-oxoheptyl)-3,7-diazabicyclo[3.3.1]nonane-3-carboxamide;

7-[(2R)-3-(4-cyanophenoxy)-2-hydroxypropyl]-N-(2-propynyl)-3,7-diazabicyclo[3.3.1]nonane-3-carboxamide;

7-(3-{4-cyano-2-[(cyclopropylamino)carbonyl]phenoxy}-2-hydroxy-propyl)-N-propionyl-3,7-diazabicyclo[3.3.1]nonane-3-carboxamide;

7-[(2S)-3-(4-cyanophenoxy)-2-hydroxypropyl]-N-phenyl-3,7-diaza-bicyclo[3.3.1]nonane-3-carboxamide;

7-[(2R)-3-(4-cyanophenoxy)-2-hydroxypropyl]-N-(cyclopropylmethyl)-3,7-diazabicyclo[3.3.1]nonane-3-carboxamide;

tert-butyl (1R)-2-(4-cyanophenoxy)-1-({7-[(propionylamino)carbonyl]-3,7-diazabicyclo[3.3.1]non-3-yl}methyl)ethylcarbamate;

7-(3-{4-cyano-2-[(iso-propylamino)carbonyl]phenoxy}-2-hydroxypropyl)-N-ethyl-3,7-diazabicyclo[3.3.1]nonane-3-carboxamide;

tert-butyl 2-(4-cyanophenoxy)-1-({7-[(propionylamino)carbonyl]-3,7-diazabicyclo[3.3.1]non-3-yl}methyl)ethylcarbamate;

tert-butyl 2-(4-cyanophenoxy)-1-({7-[(iso-propylamino)carbonyl]-3,7-diazabicyclo[3.3.1]non-3-yl }methyl)ethyl(methyl)carbamate;

7-[3-(4-cyanophenoxy)-2-(methylamino)propyl]-N-iso-propyl-3,7-diazabicyclo[3.3.1]nonane-3-carboxamide;

7-{3-(4-cyanophenoxy)-2-[methyl(methylsulfonyl)amino]propyl}-N-iso-propyl-3,7-diazabicyclo[3.3.1]nonane-3-carboxamide;

N-(tert-butyl)-7-[3-(4-cyanophenoxy)-2-hydroxypropyl]-3,7-diazabicyclo-[3.3.1]nonane-3-carboxamide;

7-[2-amino-3-(4-cyanophenoxy)propyl]-3,7-diazabicyclo[3.3.1]nonane-3-carboxamide;

tert-butyl 2-[7-(aminocarbonyl)-3,7-diazabicyclo[3.3.1]non-3-yl]-1-[(4-cyanophenoxy)methyl]ethylcarbamate;

tert-butyl 2-(4-cyanophenoxy)-1-({7-[(tetrahydro-2H-pyran-2-ylamino)-carbonyl]-3,7-diazabicyclo[3.3.1]non-3-yl}methyl)ethylcarbamate;

N-(4-cyanophenyl)-7-(4-oxoheptyl)-3,7-diazabicyclo[3.3.1]nonane-3-carboxamide; -carboxamide;

7-[3-(4-cyanophenoxy)-2-hydroxypropyl]-N-(2,2-dimethylpropanoyl)-3,7-diazabicyclo [3.3.1]nonane-3-carboxamide;

N-(tert-butoxy)-7-[3-(4-cyanophenoxy)-2-hydroxypropyl]-3,7-diazabicyclo[3.3.1]nonane-3-carboxamide;

2-[7-(aminocarbonyl)-3,7-diazabicyclo[3.3.1]non-3-yl]-1-[(4-cyano-2-methylphenoxy)methyl]ethyl tert-butylcarbamate;

7-[(2S)-3-(4-cyanophenoxy)-2-hydroxypropyl]-N-iso-propyl-N-methyl-3,7-diazabicyclo[3.3.1]nonane-3-carboxamide;

N-(4-cyanophenethyl)-7-iso-propyl-3,7-diazabicyclo[3.3.1]nonane-3-carboxamide;

N-(tert-butoxy)-7-[3-(4-cyanophenoxy)-2-hydroxypropyl]-N-methyl-3,7-diazabicyclo[3.3.1]nonane-3-carboxamide;

N-(4-cyanophenethyl)-7-(3,4-dimethoxyphenethyl)-3,7-diazabicyclo-[3.3.1]nonane-3-carboxamide;

7-[3-(4-cyanophenoxy)-2-hydroxypropyl]-N-cyclopropyl-3,7-diazabicyclo-[3.3.1]nonane-3-carboxamide;

7-[2-amino-3-(4-cyanophenoxy)propyl]-N-(tert-butyl)-3,7-diazabicyclo-[3.3.1]nonane-3-carboxamide;

N-[3-(4-cyanophenoxy)propyl]-7-[5-(ethylamino)-5-oxopentyl]-3,7-diazabicyclo[3.3.1]nonane-3-carboxamide;

7-[3-(4-cyanophenoxy)-2-hydroxypropyl]-N-(3,5-dimethyl-4-isoxazolyl)-3,7-diazabicyclo[3.3.1]nonane-3-carboxamide;

7-[3-(4-cyanoanilino)propyl]-N-iso-propyl-3,7-diazabicyclo[3.3.1]nonane-3-carboxamide;

7-[4-(4-cyanophenyl)-4-hydroxybutyl]-N-ethyl-3,7-diazabicyclo[3.3.1]-nonane-3-carboxamide;

ethyl {7-[3-(4-cyanophenoxy)-2-hydroxypropyl]-3,7-diazabicyclo[3.3.1]-non-3-yl}carbonylcarbamate;

7-[3-(4-cyanophenoxy)-2-hydroxypropyl]-N-(2,6-dimethoxyphenyl)-3,7-diazabicyclo[3.3.1]nonane-3-carboxamide;

7-[3-(4-cyanophenoxy)-2-hydroxypropyl]-N-(4-cyanophenyl)-3,7-diazabicyclo[3.3.1]nonane-3-carboxamide;

N-benzyl-7-[3-(4-cyanophenoxy)-2-hydroxypropyl]-3,7-diazabicyclo-[3.3.1]nonane-3-carboxamide;

7-[3-(4-cyanophenoxy)-2-hydroxypropyl]-N-hexyl-3,7-diazabicyclo[3.3.1]-nonane-3-carboxamide;

ethyl 3-[({7-[3-(4-cyanophenoxy)-2-hydroxypropyl]-3,7-diazabicyclo-[3.3.1]non-3-yl}carbonyl)amino]propanoate;

N-(4-butoxyphenyl)-7-[3-(4-cyanophenoxy)-2-hydroxypropyl]-3,7-diazabicyclo[3.3.1]nonane-3-carboxamide;

7-[3-(4-cyanophenoxy)-2-hydroxypropyl]-N-(3-cyanophenyl)-3,7-diazabicyclo[3.3.1]nonane-3-carboxamide;

7-[3-(4-cyanophenoxy)-2-hydroxypropyl]-N-(3,4-dimethoxyphenyl)-3,7-diazabicyclo[3.3.1]nonane-3-carboxamide;

butyl 2-[({7-[3-(4-cyanophenoxy)-2-hydroxypropyl]-3,7-diazabicyclo-[3.3.1]non-3-yl}carbonyl)amino]acetate;

7-[3-(4-cyanophenoxy)-2-hydroxypropyl]-N-(4-methoxyphenyl)-3,7-diazabicyclo[3.3.1]nonane-3-carboxamide;

7-[3-(4-cyanophenoxy)-2-hydroxypropyl]-N-(3,4-dimethoxyphenethyl)-3,7-diazabicyclo[3.3.1]nonane-3-carboxamide;

7-[3-(4-cyanophenoxy)-2-hydroxypropyl]-N-(3,4-dimethoxybenzyl)-3,7-diazabicyclo[3.3.1]nonane-3-carboxamide;

7-[3-(4-cyanophenoxy)-2-hydroxypropyl]-N-(3,4,5-trimethoxyphenyl)-3,7-diazabicyclo[3.3.1]nonane-3-carboxamide;

7-[3-(4-cyanophenoxy)-2-hydroxypropyl]-N-(3,4-dihydro-2H-1,5-benzo-dioxepin-7-yl)-3,7-diazabicyclo[3.3.1]nonane-3-carboxamide;

7-[3-(4-cyanophenoxy)-2-hydroxypropyl]-N-(2,6-dimethylphenyl)-3,7-diazabicyclo[3.3.1]nonane-3-carboxamide; -diazabicyclo[3.3.1]nonane-3-carboxamide;

iso-propyl {7-[3-(4-cyanophenoxy)-2-hydroxypropyl]-3,7-diazabicyclo-[3.3.1]non-3-yl}carbonylcarbamate;

7-[3-(4-cyanophenoxy)-2-hydroxypropyl]-N-(2-fluoroethyl)-3,7-diazabicyclo[3.3.1]nonane-3-carboxamide;

7-[3-(4-cyanophenoxy)-2-hydroxypropyl]-N-{2-[(cyclopropylmethyl)-amino]-2-oxoethyl}-3,7-diazabicyclo[3.3.1]nonane-3-carboxamide;

N-(tert-butyl)-7-{2-hydroxy-3-[(2-methyl-1-oxo-1,2-dihydro-4-isoquinolinyl)oxy]propyl}-3,7-diazabicyclo[3.3.1]nonane-3-carboxamide;

N-(1-cyano-1-methylethyl)-7-[3-(4-cyanophenoxy)-2-hydroxypropyl]-3,7-diazabicyclo[3.3.1]nonane-3-carboxamide;

7-[2-amino-3-(4-cyanophenoxy)propyl]-N-(1,3-benzodioxol-5-ylmethyl)-3,7-diazabicyclo[3.3.1]nonane-3-carboxamide;

7-[3-(4-cyanophenoxy)-2-hydroxypropyl]-N-iso-propyl-3,7-diazabicyclo-[3.3.1]nonane-3-carboxamide;

4-(3-{7-[(2,6-dimethyl-4-morpholinyl)carbonyl]-3,7-diazabicyclo[3.3.1]-non-3-yl}-2-hydroxypropoxy)benzonitrile;

N-[cyano(4-fluorophenyl)methyl]-7-[3-(4-cyanophenoxy)-2-hydroxy-propyl]-3,7-diazabicyclo[3.3.1]nonane-3-carboxamide;

N-(cyanomethyl)-7-[3-(4-cyanophenoxy)-2-hydroxypropyl]-N-methyl-3,7-diazabicyclo[3.3.1]nonane-3-carboxamide;

7-[4-(4-cyanophenoxy)-2-hydroxybutyl]-N-ethyl-3,7-diazabicyclo[3.3.1]-nonane-3-carboxamide;

7-[4-(4-cyanophenyl)butyl]-N-[2-oxo-2-(propylamino)ethyl]-3,7-diazabicyclo[3.3.1]nonane-3-carboxamide;

7-[4-(4-cyanophenyl)butyl]-N-propyl-3,7-diazabicyclo[3.3.1]nonane-3-carboxamide;

7-[2-amino-4-(4-cyanophenoxy)butyl]-N-propyl-3,7-diazabicyclo[3.3.1]-nonane-3-carboxamide;

7-[4-(4-cyanophenyl)butyl]-N-ethyl-3,7-diazabicyclo[3.3.1]nonane-3-carboxamide;

7-[3-(4-cyanophenoxy)-2-hydroxypropyl]-N-[2-(2-methoxyethoxy)ethyl]-3,7-diazabicyclo[3.3.1]nonane-3-carboxamide;

N-(4-cyanophenyl)-7-(3,3-dimethyl-2-oxobutyl)-3,7-diazabicyclo[3.3.1]-nonane-3-carboxamide;

N-(4-cyanophenyl)-7-(3,4-dimethoxyphenethyl)-3,7-diazabicyclo[3.3.1]-nonane-3-carboxamide;

N-(4-cyanophenyl)-7-(cyclopropylmethyl)-3,7-diazabicyclo[3.3.1]nonane-3-carboxamide;

N-(4-cyanophenyl)-7-[2-(2-methoxyethoxy)ethyl]-3,7-diazabicyclo[3.3.1]-nonane-3-carboxamide;

N-(4-cyanophenyl)-7-[2-(2,3-dihydro-1,4-benzodioxin-6-yl)-2-oxoethyl]-3,7-diazabicyclo[3.3.1]nonane-3-carboxamide;

7-[3-(4-acetyl-1-piperazinyl)propyl]-N-(4-cyanophenyl)-3,7-diazabicyclo-[3.3.1]nonane-3-carboxamide; and

7-[(2S)-3-(4-cyanophenoxy)-2-hydroxypropyl]-N-[2-oxo-2-(propylamino)-ethyl]-3,7-diazabicyclo[3.3.1]nonane-3-carboxamide.

### Example 37

Title compounds of the above Examples were tested in Test A above and were found to exhibit D₁₀ values of more than 6.0.

### Abbreviations

| | |
|---|---|
| AcOH = | acetic acid |
| ADDP = | 1,1′-(azodicarbonyl)dipiperidine |
| aq. = | aqueous |
| atm. = | atmospheres |
| CBz = | benzyloxycarbonyl |
| CDI = | carbonyl diimidazole |
| Bu = | butyl |
| DCM = | dichloromethane |
| DMF = | dimethylformamide |
| DMSO = | dimethylsulfoxide |
| Et = | ethyl |
| EtOAc = | ethyl acetate |
| EtOH = | ethanol |
| ESI = | electron spray interface |
| eq. = | equivalents |
| FAB = | fast atom bombardment |
| h = | hours |
| IPA = | iso-propanol |
| i-PrOH = | iso-propanol |
| LC = | liquid chromatography |
| HPLC = | high performance liquid chromatography |
| mCPBA = | meta-chloroperbenzoic acid |
| Me = | methyl |
| MeCN = | acetonitrile |
| MeOH = | methanol |
| mesyl = | methanesulfonate |
| min. = | minutes |
| Ms = | mesylate |
| MS = | mass spectroscopy |
| NADPH = | nicotinamide adenine dinucleotide phosphate, reduced |
| | form |
| NMR = | nuclear magnetic resonance |
| OSu = | O-succinyl |
| Pd/C = | palladium on carbon |
| pTSA = | para-toluenesulfonic acid |
| rt. = | room temperature |
| satd. = | saturated |
| TEA = | triethylamine |
| THF = | tetrahydrofuran |
| tlc = | thin layer chromatography |
| TMS = | tetramethylsilane |

Prefixes n-, s-, i-, iso-, t- and tert- have their usual meanings: normal, iso, secondary and tertiary.

1. A compound of formula I, 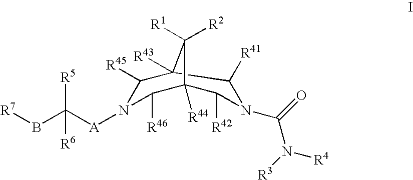
wherein
R¹ and R² independently represent H, C₁₋₄ alkyl, OR^{2b} or N(R^{2c})R^{2d}, or together form —O—(CH₂)₂—O—, —(CH₂)₃—, —(CH₂)₄— or —(CH₂)₅—;
R^{2b}, R^{2c} and R^{2d} independently represent H or C₁₋₆ alkyl;
R³ represents H, C₁₋₆ alkyl or, together with R⁴, represents C₃₋₆ alkylene (which alkylene group is optionally interrupted by an O atom and/or is optionally substituted by one or more C₁₋₃ alkyl groups);
R⁴ represents H, C₁₋₁₂ alkyl, C₁₋₆ alkoxy (which latter two groups are both optionally substituted and/or terminated by one or more substituents selected from —OH, halo, cyano, nitro, C₁₋₄ alkyl and/or C₁₋₄ alkoxy), —(CH₂)_{q}-aryl, —(CH₂)_{q}-oxyaryl, —(CH₂)_{q}-Het¹ (which latter three groups are optionally substituted (at the —(CH₂)_{q}— part and/or the aryl/Het¹ part) by one or more substituents selected from —OH, halo, cyano, nitro, —C(O)R¹⁰, —C(O)OR¹⁰, —N(H)S(O)₂R^{11a}, C₁₋₆ alkyl and/or C₁₋₆ alkoxy), —(CH₂)_{q}N(H)C(O)R⁸, —(CH₂)_{q}S(O)₂R⁸, —(CH₂)_{q}C(O)R⁸, —(CH₂)_{q}C(O)OR⁸, —(CH₂)_{q}C(O)N(R⁹)R⁸ or, together with R³, represents C₃₋₆ alkylene (which alkylene group is optionally interrupted by O atom and/or is optionally substituted by one or more C₁₋₃ alkyl groups);
q represents 0, 1, 2, 3, 4, 5 or 6;
R⁸ represents H, C₁₋₆ alkyl, aryl (which latter group is optionally substituted and/or terminated by one or more substituents selected from —OH, halo, cyano, nitro, —C(O)R¹⁰, —C(O)OR¹¹, —N(H)S(O)₂R^{11a}, C₁₋₆ alkyl and/or C₁₋₆ alkoxy) or, together with R⁹, represents C₃₋₇ alkylene;
R⁹ represents H, C₁₋₄ alkyl or, together with R⁸, represents C₃₋₇ alkylene; Het¹ represents a five to twelve-membered heterocyclic ring containing one or more heteroatoms selected from oxygen, nitrogen and/or sulfur, and which also optionally includes one or more ═O substituents;
R⁴¹, R⁴², R⁴³, R⁴⁴, R⁴⁵ or R⁴⁶ independently represent H or C₁₋₃ alkyl;
R⁵ represents H, halo, C₁₋₃ alkyl, —OR¹², —N(R¹³)R¹² or, together with R⁶, represents ═O;
R⁶ represents H, C₁₋₄ alkyl or, together with R⁵, represents ═O;
R¹² represents H, C₁₋₆ alkyl, —S(O)₂—C₁₋₄-alkyl, —C(O)R¹⁴, —C(O)OR¹⁴, —C(O)N(R¹⁵)R¹⁵a or aryl (which latter group is optionally substituted and/or terminated by one or more substituents selected from —OH, halo, cyano, nitro, —C(O)R¹⁰, —C(O)OR¹¹, —N(H)S(O)₂R^{11a}, C₁₋₆ alkyl and/or C₁₋₆ alkoxy);
R¹³ represents H or C₁₋₄ alkyl;
R¹⁴ represents H or C₁₋₆ alkyl;
R¹⁵ and R^{15a} independently represent H or C₁₋₄ alkyl, or together represent C₃₋₆ alkylene, optionally interrupted by O atom;
A represents a single bond, C₁₋₆ alkylene, —N(R¹⁶)(CH₂)ᵣ— or —O(CH₂)ᵣ— (in which two latter groups, the —(CH₂)ᵣ— group is attached to the bispidine nitrogen atom);
B represents a single bond, C₁₋₄ alkylene, —(CH₂)ₙN(R¹⁷)—, —(CH₂)ₙS(O)ₚ—, —(CH₂)ₙO— (in which three latter groups, the —(CH₂)ₙ— group is attached to the carbon atom bearing R⁵ and R⁶), —C(O)N(R¹⁷)— (in which latter group, the —C(O)— group is attached to the carbon atom bearing R⁵ and R⁶), —N(R¹⁷)C(O)O(CH₂)ₙ—, —N(R¹⁷)(CH₂)ₙ— (in which two latter groups, the N(R¹⁷) group is attached to the carbon atom bearing R⁵ and R⁶) or —(CH₂)ₘC(H)(OH)(CH₂)ₙ— (in which latter group, the —(CH₂)ₘ— group is attached to the carbon atom bearing R⁵ and R⁶);
m represents 1, 2 or 3;
n and r independently represent 0, 1, 2, 3 or 4;
p represents 0, 1 or 2;
R¹⁶ and R¹⁷ independently represent H or C₁₋₄ alkyl;
R⁷ represents C₁₋₆ alkyl, aryl or Het², all of which groups are optionally substituted and/or terminated (as appropriate) by one or more substituents selected from —OH, cyano, halo, amino, nitro, Het³, —C(O)R¹⁰, —C(O)OR¹¹, C₁₋₆ alkyl, C₁₋₆ alkoxy, —N(H)S(O)₂R¹⁸, —S(O)₂R¹⁹, —OS(O)₂R²⁰, —N(H)C(O)N(H)R²¹, —C(O)N(H)R²² and/or aryl (which latter group is optionally substituted by one or more cyano groups);
Het² and Het³ independently represent a five to twelve-membered heterocyclic group containing one or more heteroatoms selected from oxygen, nitrogen and/or sulfur, and which also optionally includes one or more ═O substituents;
R¹⁸, R¹⁹ and R²⁰ independently represent C₁₋₆ alkyl;
R²¹ and R²² independently represent H or C₁₋₆ alkyl (optionally terminated by cyano); and
R¹⁰ and R¹¹ independently represent, at each individual occurrence, H or C₁₋₆ alkyl;
R^{11a} represents, at each individual occurrence, C₁₋₆ alkyl; or a pharmaceutically acceptable derivative thereof;
provided that:
(a) when A and B are both single bonds and R⁷ is optionally substituted aryl, then R⁵ and R⁶ do not both represent H;
(b) when A represents a single bond, then R⁵ and R⁶ do not together represent═O; and
(c) when R⁵ represents —OR¹² or —N(R¹³)R¹², then:
(i) A does not represent —N(R¹⁶)(CH₂)ᵣ— or —O(CH₂)ᵣ—; and/or
(ii) n does not represent 0 when B represents —(CH₂)ₙN(R¹⁷)—, —(CH₂)ₙS(O)ₚ— or —(CH₂)ₙO—.

2. A compound as claimed in claim 1, wherein R¹ represents H.

3. A compound as claimed in claim 1, wherein R² represents H.

4. A compound as claimed in claim 1, wherein R³ represents H; C₁₋₂ alkyl; or, together with R⁴ represents C₄₋₅ alkylene, optionally interrupted by O atom and/or optionally substituted by one or more methyl groups.

5. A compound as claimed in claim 4, wherein R³ represents H.

6. A compound as claimed in claim 1, wherein R⁴ represents H; linear or branched and/or saturated or unsaturated and/or cyclic, acyclic and/or part cyclic/acyclic C₁₋₈ alkyl (which alkyl group is optionally substituted by one or more cyano or halo groups and/or interrupted by an O atom); C₁₋₆ alkoxy; —(CH₂)_{q}S(O)₂R⁸, —(CH₂)_{q}C(O)OR⁸, —(CH₂)_{q}N(H)C(O)R⁸, —(CH₂)_{q}C(O)R⁸, (in which latter four groups, q represents 0, 1 or and R⁸ represents linear or branched and/or acyclic, cyclic and/or part cyclic/acyclic C₁₋₄ alkyl, or phenyl (which phenyl group is optionally substituted by one or more cyano and/or C₁₋₃ alkyl groups));
—(CH₂)_{q}C(O)N(R⁹)R⁸ (in which latter group, q represents 0, 1 or 2 and R⁸ and R⁹ independently represent H, linear or branched and/or acyclic, cyclic and/or part cyclic/acyclic C₁₋₄ alkyl, or together represent C₄₋₆ alkylene);
—(CH₂)_{q}-phenyl, —(CH₂)_{q}-oxyphenyl or —(CH₂)_{q}-Het¹ (in which latter three groups, q represents 0, 1, 2 or 3, the —(CH₂)_{q}— part is optionally substituted by a cyano group, and the phenyl, or Het¹, part is optionally substituted with one or more substituents selected from cyano, nitro, linear or branched C₁₋₄ alkyl, linear or branched C₁₋₄ alkoxy and N(H)S(O)₂R^{11a}); or, together with R³, represents C₄₋₅ alkylene, optionally interrupted by an O atom and/or optionally substituted by one or more methyl groups.

7. A compound as claimed in claim 1, wherein R⁵ represents H; fluoro; OR¹² (in which R¹² represents H, phenyl (optionally substituted by one or more methoxy groups) or C(O)N(H)R^{15a} (in which R^{15a} represents linear or branched C₁₋₄ alkyl)); —N(R¹³)(R¹²) (in which R¹² represents H, C₁₋₂ alkyl, —S(O)₂—C₁₋₂ alkyl, —C(O)R¹⁴ (in which R¹⁴ represents C₁₋₂ alkyl), —C(O)OR¹⁴ (in which R¹⁴ represents linear or branched C₁₋₅ alkyl) or —C(O)N(R¹⁵)(R^{15a}) (in which R¹⁵ and R^{15a} independently represent H or linear or branched C₁₋₃ alkyl or together represent C₄₋₅ alkylene, which alkylene group is optionally interrupted by O atom) and R¹³ represents H or C₁₋₂ alkyl); or, together with R⁶, represents ═O.

8. A compound as claimed in claim 7, wherein R⁵ represents H, OH or —N(H)C(O)N(R¹⁵)(R^{15a}).

9. A compound as claimed in claim 1, wherein R⁶ represents H or C₁₋₂ alkyl or together with R⁵ represents ═O.

10. A compound as claimed in claim 9, wherein R⁶ represents H.

11. A compound as claimed in claim 1, wherein A represents a single bond, linear or branched C₁₋₄ alkylene (which group is also optionally interrupted by O), —N(H)(CH₂)ᵣ— or —O(CH₂)ᵣ— (in which latter two cases r is 1 or 2).

12. A compound as claimed in claim 11, wherein A represents —CH₂— or —(CH₂)₂—.

13. A compound as claimed in claim 1, wherein B represents a single bond, C₁₋₄ alkylene, —(CH₂)ₙO—, —(CH₂)ₙS(O)₂—, —(CH₂)ₙN(H)— or —N(H)(CH₂)ₙ— (in which latter four cases n is 0, 1, 2 or 3).

14. A compound as claimed in claim 13, wherein B represents a single bond, —CH₂N(H)— or —CH₂O—.

15. A compound as claimed in claim 1, wherein R⁷ represents linear or branched and/or acyclic, cyclic and/or part cyclic/acyclic C₁₋₆ alkyl (optionally substituted and/or terminated by OH); Het² (optionally substituted by one or more substituents selected from cyano, C₁₋₃ alkyl, phenyl (which latter group is optionally substituted with one or more cyano groups), ═O, C(O)R¹⁰ (in which R¹⁰ is linear or branched C₁₋₃ alkyl) or S(O)₂R¹⁹ (in which R¹⁹ is C₁₋₂ alkyl)); or phenyl (optionally substituted by one or more substituents selected from cyano, nitro, linear or branched C₁₋₃ alkyl, linear or branched C₁₋₃ alkoxy, fluoro, chloro, C(O)N(H)R²² (in which R²² represents linear or branched and/or acyclic, cyclic and/or part cyclic/acyclic C₁₋₄ alkyl, which alkyl group is optionally terminated by cyano), N(H)S(O)₂R¹⁸ (in which R¹⁸ represents C₁₋₂ alkyl) or Het³).

16. A compound as claimed in claim 15, wherein R⁷ represents phenyl (substituted by a cyano group (preferably in the 4-position relative to B) and by one or more optional C(O)N(H)R²² substituent).

17. A compound as claimed in claim 1, wherein R⁴¹, R⁴², R⁴³, R⁴⁴, R⁴⁵ and R⁴⁶ all represent H.

18. A pharmaceutical formulation including a compound as defined in claim 1 in admixture with a pharmaceutically-acceptable adjuvant, diluent or carrier.

19. A pharmaceutical formulation for use in the prophylaxis or the treatment of an arrhythmia, comprising a compound as defined in claim 1.

20. A compound as defined in claim 1 for use as a pharmaceutical.

21. A compound as defined in any one of claims 1 to 17 claim 1 for use in the prophylaxis or the treatment of an arrhythmia.

22. The use of a compound as defined in claim 1 as active ingredient in the manufacture of a medicament for use in the prophylaxis or the treatment of an arrhythmia.

23. The use as claimed in claim 22, wherein the arrhythmia is an atrial or a ventricular arrhythmia.

24. A method of prophylaxis or treatment of an arrhythmia which method comprises administration of a therapeutically effective amount of a compound as defined in claim 1 to a person suffering from, or susceptible to, such a condition.

25. A process for the preparation of a compound of formula I as defined in claim 1 which comprises:
(a) for compounds of formula I in which R³ is H, reaction of a compound of formula II, 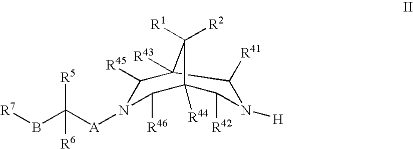
wherein R¹, R², R⁵, R⁶, R⁷, R⁴¹, R⁴², R⁴³, R⁴⁴, R⁴⁵, R⁴⁶A and B are as defined in claim 1 with a compound of formula III,
R⁴—N═C═O III
wherein R⁴ is as defined in claim 1;
(b) reaction of a compound of formula II, as defined above, with a carbonic acid derivative of formula IV,
(R³)(R⁴)NC(O)-L¹ IV
wherein L¹ represents a leaving group and R³ and R⁴ are as defined in claim 1;
(c) reaction of a compound of formula V, 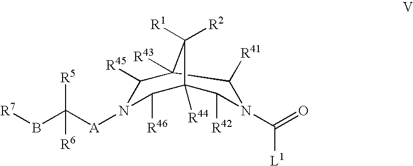
wherein and L¹ is as defined above and R¹, R², R⁵, R⁶, R⁷, R⁴¹, R⁴², R⁴³, R⁴⁴, R⁴⁵, R⁴⁶, A and B are as defined in claim 1, with a compound of formula VA,
(R³)(R⁴)NH VA
wherein R³ and R⁴ are as defined in claim 1;
(d) for compounds of formula I in which A represents CH₂ and R⁵ represents —OH or —N(H)R¹² , reaction of a compound of formula VI, 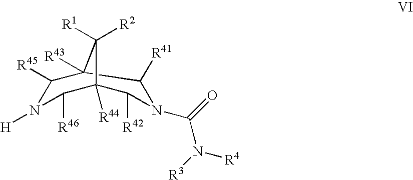
wherein R¹, R², R³, R⁴, R⁴¹, R⁴², R⁴³, R⁴⁴, R⁴⁵ and R⁴⁶ are as defined in claim 1, with a compound of formula VII, 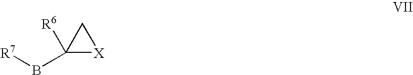
wherein X represents O or N(R¹²) and R⁶, R⁷, R¹² and B are as defined in claim 1;
(e) reaction of a compound of formula VI, as defined above, with a compound of formula VIII, 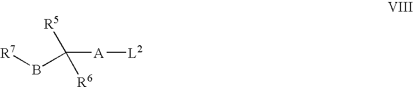
wherein L² represents a leaving group and R⁵, R⁶, R⁷, A and B are as defined in claim 1;
(f) for compounds of formula I in which R⁵ represents H or OH and R⁶ represents H, reduction of a compound of formula IX, 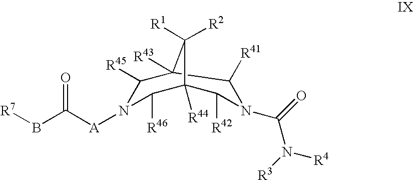
wherein R¹, R², R³, R⁴, R⁷, R⁴¹,R⁴², R⁴³, R⁴⁴, R⁴⁵, R⁴⁶, A and B are as defined in claim 1;
(g) for compounds of formula I in which one of R¹ and R² represents H or OH and the other represents H, reduction of a corresponding compound of formula X, 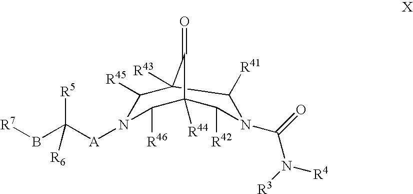
wherein R³, R⁴, R⁵, R⁶, R⁷, R⁴¹, R⁴², R⁴³, R⁴⁴, R⁴⁵, R⁴⁶, A and B are as defined in claim 1;
(h) for compounds of formula I in which R¹ and R² together represent —O(CH₂)₂O—, reaction of a corresponding compound of formula X as defined above with ethane-1,2-diol;
(i) for compounds of formula I in which B represents —(CH₂)ₙO—, reaction of a compound of formula XI, 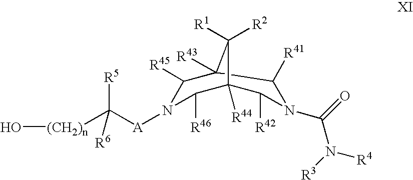
wherein R¹, R², R³, R⁴, R⁵, R⁶, R⁴¹, R⁴², R⁴³, R⁴⁴, R⁴⁵, R⁴⁶, A and n are as defined in claim 1, with a compound of formula XIA,
R⁷OH XIA
in which R⁷ is as defined in claim 1;
(j) for compounds of formula I which are bispidine-nitrogen N-oxide derivatives, oxidation of the corresponding bispidine nitrogen of a corresponding compound of formula I;
(k) for compounds of formula I which are C₁₋₄ alkyl quaternary ammonium salt derivatives, in which the alkyl group is attached to a bispidine nitrogen, reaction, at the bispidine nitrogen, of a corresponding compound of formula I with a compound of formula XII,
R^{b}L³ XII
wherein R^{b} represents C₁₋₄ alkyl and L³ is a leaving group;
(I) for compounds of formula I in which R⁵ and R⁶ represent H, A represents C₁₋₆ alkylene and B represents —N(R¹⁷)(CH₂)ₙ—, reaction of a compound of formula XIII, 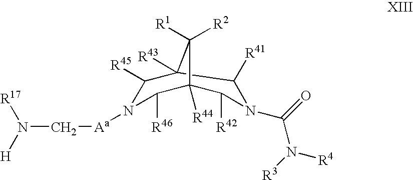
wherein A^{a} represents C₁₋₆ alkylene and R¹, R², R³, R⁴, R⁴¹, R⁴², R⁴³, R⁴⁴, R⁴⁵, R⁴⁶ and R⁴⁷ are as defined in claim 1 with a compound of formula XIV,
R⁷—(CH₂)ₙ-L² XIV
wherein L² is as defined above and R⁷ and n are as defined in claim 1; (m) for compounds of formula I in which R⁵ represents —NH₂, reduction of a corresponding compound of formula XV, 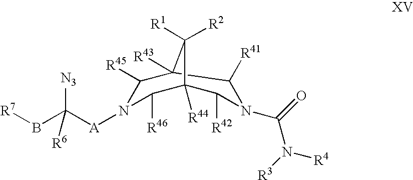
wherein R¹, R², R³, R⁴, R⁶, R⁷, R⁴¹, R⁴², R⁴³, R⁴⁴, R⁴⁵, R⁴⁶, A and B are as defined in claim 1;
(n) for compounds of formula I in which R⁵ represents
—N(R¹³)C(O)NH(R¹⁵), reaction of a corresponding compound of formula I in which R⁵ represents —N(R¹³)H with a compound of formula XVI,
R¹⁵N═C═O XVI
wherein R¹⁵ is as defined in claim 1;
(o) for compounds of formula I in which R⁵ represents —N(R¹³)C(O)R¹⁴, reaction of a corresponding compound of formula I in which R⁵ represents —N(R¹³)H with a compound of formula XVII,
R¹⁴C(O)R^{x} XVII
wherein R^{x} represents a suitable leaving group and R¹⁴ is as defined in claim 1;
(p) for compounds of formula I in which R⁵ represents —N(H)R¹², wherein R¹² is as defined in claim 1 provided that it does not represent H, reaction of a corresponding compound of formula I, in which R⁵ represents —NH₂ with a compound of formula XVIII,
R^{12a}L¹ XVIII
wherein R^{12a} represents R¹² as defined in claim 1 provided that it does not represent H and L¹ is as defined above;
(q) for compounds of formula I in which R⁵ represents —OR¹² in which R¹² represents C₁₋₆ alkyl or optionally substituted aryl, reaction of a corresponding compound of formula I in which R⁵ represents —OH with a compound of formula XIX,
R^{12a}OH XIX
wherein R^{12a} represents C₁₋₆ alkyl or optionally substituted aryl;
(r) for compounds of formula I in which R⁵ represents —OR¹², in which R¹² represents C₁₋₆ alkyl or optionally substituted aryl, reaction of a compound of formula XX, 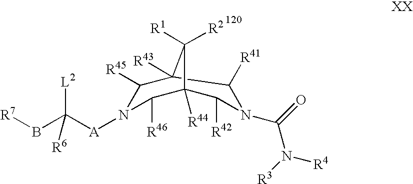
wherein L² is as defined above and R¹, R², R³, R⁴, R⁶, R⁷, R⁴¹, R⁴², R⁴³, R⁴⁴, R⁴⁵, R⁴⁶, A and B are as defined in claim 1 with a compound of formula XIX as defined above;
(s) for compounds of formula I in which R⁵ represents OR¹² and R¹² represents C(O)R¹⁴, reaction of a corresponding compound of formula I in which R⁵ represents OH with a compound of formula XXI,
R¹⁴CO₂H XXI
wherein R¹⁴ is as defined in claim 1;
(t) for compounds of formula I in which R⁵ represents halo, substitution of a corresponding compound of formula I in which R⁵ represents —OH, using an appropriate halogenating agent;
(u) for compounds of formula I in which R³ and/or R⁴ as appropriate represent alkyl groups, alkylation of a corresponding compound of formula I, in which R³ and/or R⁴ (as appropriate) represent H;
(v) conversion of one R⁴ group to another;
(w) for compounds of formula I in which one of R² and R³ represents —NH₂ and the other represents H, reduction of a compound of formula XXIA, 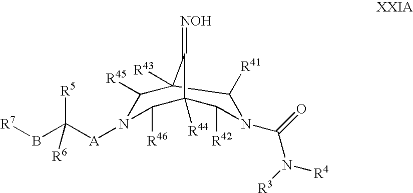
wherein R³, R⁴, R⁵, R⁶, R⁷, R⁴¹, R⁴², R⁴³, R⁴⁴, R⁴⁵, R⁴⁶, A and B areas defined in claim 1;
(x) for compounds of formula I in which one or both of R¹ and R² represent —N(R^{2c})R^{2d} in which one or both of R^{2c} and R^{2d} represents C₁₋₆ alkyl, alkylation of a corresponding compound of formula I in which R¹ and/or R² represent —N(R^{2c})R^{2d} (as appropriate) in which R^{2c} and/or R^{2d} (as appropriate) represent H, using a compound of formula XXIB,
R^{2e}L¹ XXIB
wherein R^{2e} represents C₁₋₆ alkyl and L¹ is as defined above;
(y) conversion of one substituent on R⁷ to another; or
(z) deprotection of a protected derivative of a compound of formula I as defined in claim 1.

26. A compound of formula II, as defined in claim 25, or a protected derivative thereof, provided that R⁷ does not represent optionally substituted phenyl.

27. A compound of formula V, as defined in claim 25, or a protected derivative thereof, provided that R⁷ does not represent optionally substituted phenyl.

28. A compound of formula X as defined in claim 25, or a protected derivative thereof.

29. A compound of formula XI as defined in claim 25, or a protected derivative thereof.

30. A compound of formula XIII, as defined in claim 25, or a protected derivative thereof.

31. A compound of formula XV, as defined in claim 25, or a protected derivative thereof.

32. A compound of formula XX, as defined in claim 25, or a protected derivative thereof.

33. A compound of formula XXIII, 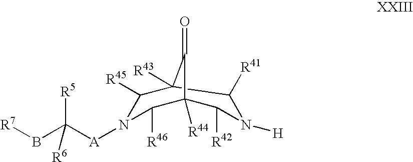
wherein R⁵, R⁶, R⁷, R⁴¹, R⁴², R⁴³, R⁴⁴, R⁴⁵, R⁴⁶, A and B are as defined in claim 1, or a protected derivative thereof.

34. A compound of formula XXV, 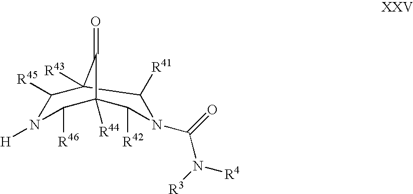
wherein R³, R⁴, R⁴¹, R⁴², R⁴³, R⁴⁴, R⁴⁵ and R⁴⁶ are as defined in claim 1, or a protected derivative thereof.

35. A process for the preparation of a compound of formula X, of formula XXIII, or of formula XXV (in which, in all cases, R⁴⁵ and R⁴⁶ both represent H), which comprises (as appropriate) reaction of either:
(i) a compound of formula XXXV, 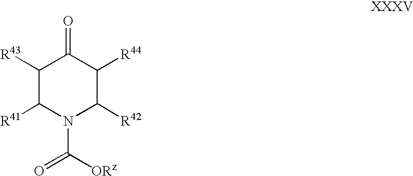
wherein R^{z} represents C₁₋₁₀ alkyl or C₁₋₃ alkylaryl and R⁴¹, R⁴², R⁴³ and R⁴⁴ are as defined in claim 1, or
(ii) 4-piperidone (or a protected derivative thereof), with (as appropriate) either:
(1) a compound of formula XXXVI,
R⁷—B—C(R⁵)(R⁶)-A-NH₂ XXXVI
wherein R⁵, R⁶, R⁷, A and B are as defined in claim 1, or
(2) NH₃ (or a protected derivative thereof),
in all cases in the presence of a formaldehyde and, in the case of compounds of formulae X and XXV, followed by conversion of the C(O)OR^{z} group in the resultant intermediate to a C(O)N(R³)(R⁴) group.

36. A process as claimed in claim 35, in which the reaction is carried out in the presence of an organic acid.

37. A process as claimed in claim 36, in which the organic acid is acetic acid.
